(19) United States
(12) Patent Application Publication
(10) Pub. No.: US 2022/0409293 A1
(43) Pub. Date: Dec. 29, 2022

(54) IMPEDANCE-BASED CHARACTERIZATION OF INTRACARDIAC STRUCTURE

(71) Applicant: Navix International Limited, Road Town, Tortola (VG)

(72) Inventors: Shlomo BEN-HAIM, Milan (IT); Andrew ADLER, Ottawa (CA); Symon STOWE, Almonte (CA)

(73) Assignee: Navix International Limited, Road Town, Tortola (VG)

(21) Appl. No.: 17/801,250

(22) PCT Filed: Feb. 18, 2021
(86) PCT No.: PCT/IB2021/051394

§ 371 Date: Aug. 21, 2022

Related U.S. Application Data
(63) Continuation-in-part of application No. PCT/IB2020/060167, filed on Oct. 29, 2020.
(60) Provisional application No. 63/118,665, filed on Nov. 26, 2020.
(60) Provisional application No. 63/048,706, filed on Jul. 7, 2020.
(60) Provisional application No. 63/015,695, filed on Apr. 27, 2020.
(60) Provisional application No. 62/978,894, filed on Feb. 20, 2020.
(60) Provisional application No. 62/978,894, filed on Feb. 20, 2020.
(60) Provisional application No. 62/927,712, filed on Oct. 30, 2019.

(51) Int. Cl. A61B 34/20; A61B 5/0538; A61B 5/287; A61F 2/24

(52) U.S. Cl. CPC A61B 34/20; A61B 5/0538; A61B 5/287; A61F 2/2466; A61B 2034/2053; A61B 2017/00703

(57) ABSTRACT

Methods and devices using measurements of heart electrophysiological activity to guide structural heart disease interventions. In some embodiments, measurements of heart electrophysiological activity are mapped into locations of a heart model defined by one or more additional measurement modalities. In some embodiments, the additional measurement modalities comprise impedance measurements. Locations to map electrophysiological data to, in some embodiments, are determined by non-electrophysiological measurements simultaneous with the electrophysiological data measurement which locate a probe—for example, measurements made by the probe itself, and/or measurements which themselves indicate positioning of the probe.

### RELATED APPLICATIONS

This application claims the benefit of priority under 35 USC § 119 of U.S. Provisional Patent Application No. 62/978,894, filed Feb. 20, 2020; U.S. Provisional Patent Application No. 63/015,695, filed Apr. 27, 2020; U.S. Provisional Patent Application No. 63/048,706, filed Jul. 7, 2020; U.S. Provisional Patent Application No. 63/118,665, filed Nov. 26, 2020 and International Patent Application No. PCT/IB2020/060167, filed Oct. 29, 2020; the contents of which are incorporated herein by reference in their entirety.

### FIELD AND BACKGROUND OF THE INVENTION

The present invention, in some embodiments thereof, relates to the field of navigation within body cavities by intrabody devices, and more particularly, to guidance of the placement of intrabody devices, optionally including implantable devices. Several medical procedures in cardiology and other medical fields comprise the use of intrabody devices such as catheter probes to reach tissue targeted for diagnosis and/or treatment while minimizing procedure invasiveness. Early imaging-based techniques (such as fluoroscopy) for navigation of the catheter and monitoring of treatments continue to be refined, and are now joined by techniques and systems such as the use of electrical field measurement-guided position sensing systems.

A variety of catheter-delivered intrabody devices are in current use for purposes of treatment and/or diagnosis, including implantable pacemakers, stents, implantable rings, implantable valve replacements (such as: aortic valve replacement, mitral valve replacement and tricuspid valve replacement), left atrial appendage (LAA) occluders, and/or atrial septal defect (ASD) occluders.

Methods of locating an intrabody catheter based on electrical measurements include, for example, International Patent Publication Nos. WO2019/035023 and WO2019/034944; the contents of which are included herein by reference in their entirety.

### SUMMARY OF THE INVENTION

According to an aspect of some embodiments of the present disclosure, there is provided a system configured to identify structures of a body lumen for guidance of procedure actions within the body lumen, the system including a processor and memory storing instructions, wherein the processor operates according to the instructions to: access measurements of: impedance, measured using by one or more electrodes positioned within the heart, and an electrophysiological signal indicative of electrical activity of heart tissue; process the impedance to identify tissue according to the positioning of the one or more electrodes, using the electrophysiological signal; and provide the identification as output for guidance of the procedure actions.

According to some embodiments of the present disclosure, the processor processes the impedance using timing information in the electrophysiological signal.

According to some embodiments of the present disclosure, the processor processes the impedance using positional information indicated by the electrophysiological signal.

According to some embodiments of the present disclosure, the processor provides the identification as output in the form of a portion of an image displayed on the display.

According to some embodiments of the present disclosure, the processor also: operates according to the instructions to access measurements of spatial position of the one or more electrodes while the impedance was measured; and uses the measurements of spatial position together with the impedance and the electrophysiological signal to identify the tissue.

According to some embodiments of the present disclosure, the impedance is indicative of motion of the identified tissue.

According to some embodiments of the present disclosure, the identified tissue includes tissue of a heart lumenal wall identified based on the indications of its movement in the impedance.

According to some embodiments of the present disclosure, the indications of heart lumenal wall movement are identified based further on relative timing of the electrophysiological signal and the impedance.

According to some embodiments of the present disclosure, the identification categorizes the heart lumenal wall according to which wall of a heart chamber the at least one electrode is in contact with.

According to some embodiments of the present disclosure, the identification distinguishes interatrial septal wall from lumenal wall adjacent to the aorta.

According to some embodiments of the present disclosure, the impedance is indicative of motion of the identified tissue.

According to some embodiments of the present disclosure, the identified tissue includes tissue of a heart valve leaflet, identified based on indications of motion of the leaflet in the impedance.

According to some embodiments of the present disclosure, the indications of motion of the heart valve leaflet are characterized using relative timing of the electrophysiological signal and the impedance.

According to some embodiments of the present disclosure, the indications of motion of the heart valve leaflet are characterized using positioning of the one or more electrodes relative to a region identified based on the electrophysiological signal.

According to some embodiments of the present disclosure, the region identified based on the electrophysiological signal is identified based on its position relative to a heart valve.

According to some embodiments of the present disclosure, the processor uses positioning of the one or more electrodes relative to a region identified based on the electrophysiological signal to select impedance measurements for use in the identification.

According to an aspect of some embodiments of the present disclosure, there is provided a method of identifying structures of a body lumen for guidance of procedure actions within the body lumen, the method including: accessing measurements of: impedance, measured by one or more electrodes positioned within the heart, and a electrophysiological signal indicative of electrical activity of heart tissue; processing the impedance to identify tissue according to the positioning of the one or more electrodes, using the electrophysiological signal; and providing the identification as output for guidance of the procedure actions.

According to some embodiments of the present disclosure, the processing uses timing information in the electrophysiological signal.

According to some embodiments of the present disclosure, the processing uses positional information indicated by the electrophysiological signal.

According to some embodiments of the present disclosure, the method includes providing the identification as output in the form of a portion of an image displayed on the display.

According to some embodiments of the present disclosure, the method includes: accessing measurements of spatial position of the one or more electrodes while the impedance was measured; and using the measurements of spatial position together with the impedance and the electrophysiological signal to identify the tissue.

According to some embodiments of the present disclosure, the impedance is indicative of motion of the identified tissue.

According to some embodiments of the present disclosure, the identified tissue includes tissue of a heart lumenal wall identified based on the indications of its movement in the impedance.

According to some embodiments of the present disclosure, the indications of heart lumenal wall movement are identified based further on relative timing of the electrophysiological signal and the impedance.

According to some embodiments of the present disclosure, the identification categorizes the heart lumenal wall according to which wall of a heart chamber the at least one electrode is in contact with.

According to some embodiments of the present disclosure, the identification distinguishes interatrial septal wall from lumenal wall adjacent to the aorta.

According to some embodiments of the present disclosure, the identified tissue includes tissue of a heart valve leaflet identified based on the indications of its movement in the impedance.

According to some embodiments of the present disclosure, the indications of heart valve leaflet movement are characterized using relative timing of the electrophysiological signal and the impedance.

According to some embodiments of the present disclosure, the indications of heart valve leaflet movement are characterized using positioning of the one or more electrodes relative to a region identified based on the electrophysiological signal.

According to some embodiments of the present disclosure, the region identified based on the electrophysiological signal is identified based on its position relative to a heart valve.

According to some embodiments of the present disclosure, the method includes using the positioning of the one or more electrodes relative to a region identified based on the electrophysiological signal to select impedance measurements for use in the identification.

According to an aspect of some embodiments of the present disclosure, there is provided a system configured to select impedance measurements suitable for analysis of movement of an intralumenal structure, the system including a processor and memory storing instructions, wherein the processor operates in accordance with the instructions to: access intracardiac electrophysiological signals measured along a range of intralumenal electrode positions; access spatial locations of the intralumenal electrode positions; access impedance measurements obtained from intralumenal electrode positions within the heart, including impedance measurements indicative of movements of the intralumenal structure; identify a selection region including intralumenal electrode positions from which at least some of the impedance measurements indicative of movements of the intralumenal structure were obtained, the identification being based on the electrophysiological signal measurements and the spatial locations; and select the impedance measurements obtained from within the selection region.

According to some embodiments of the present disclosure, the intralumenal structure includes leaflets of a heart valve.

According to some embodiments of the present disclosure, the range of intralumenal electrode positions is distributed along an imaginary atrioventricular axis.

According to some embodiments of the present disclosure, the processor further operates in accordance with the instructions to characterize proximity of the moving structure to the region.

According to some embodiments of the present disclosure, the processor further operates in accordance with the instructions to characterize changes in the intralumenal structure over time.

According to some embodiments of the present disclosure, the changes in the intralumenal structure are characterized using impedance measurement amplitude as a marker for positions of the intralumenal structure.

According to some embodiments of the present disclosure, the changes in the intralumenal structure are characterized using a threshold setting for an amplitude of the impedance measurements.

According to some embodiments of the present disclosure, the processor also operates in accordance with the instructions to compare pre-intervention and post-intervention maps of the structure to identify changes.

According to some embodiments of the present disclosure, the impedance measurements are made concurrently with the electrophysiological signal measurements.

According to an aspect of some embodiments of the present disclosure, there is provided a method of mapping valve leaflets, including: positioning an electrode in a plurality of different positions adjacent to a valve plane of a cardiac valve; measuring impedances at the plurality of different positions, using the electrode; and applying a threshold criterion to the measured impedance, to identify positioning of leaflets of the cardiac valve with respect to the electrode positions at which the impedance was measured.

According to an aspect of some embodiments of the present disclosure, there is provided a system configured to image structures of a body lumen for guidance of procedure actions within the body lumen, the system including a processor and memory storing instructions, wherein the processor operates according to the instructions to: access measurements of: impedance measured by one or more electrodes positioned within the heart, and an electrophysiological signal indicative of electrical activity of heart tissue; and process the impedance measurements and the electrophysiological signal to image structure of the body lumen according to the positioning of the one or more electrodes.

According to some embodiments of the present disclosure, the processor processes the impedance measurements using timing information in the electrophysiological signal to image the structure.

According to some embodiments of the present disclosure, the processor processes the impedance measurements using positional information indicated by the electrophysiological signal to image the structure.

According to some embodiments of the present disclosure, the processor provides the structure imaging results as an image displayed on the display.

According to some embodiments of the present disclosure, the processor also: operates according to the instructions to access measurements of spatial position of the one or more electrodes while the impedance measurements were measured; and uses the measurements of spatial position together with the impedance measurements and the electrophysiological signal to image the structure.

According to some embodiments of the present disclosure, the impedance measurements are indicative of motion of the identified tissue.

According to some embodiments of the present disclosure, the imaged structure includes tissue of a heart valve leaflet.

According to an aspect of some embodiments of the present disclosure, there is provided a system configured to identify structures of a body lumen for guidance of procedure actions within the body lumen, the system including a processor and memory storing instructions, wherein the processor operates according to the instructions to: access measurements of: artificially applied electrical signals, measured using by one or more electrodes positioned within the heart, and an electrophysiological signal indicative of electrical activity of heart tissue; process the impedance to identify tissue according to the positioning of the one or more electrodes, using the electrophysiological signal; and provide the identification as output for guidance of the procedure actions.

According to an aspect of some embodiments of the present disclosure, there is provided a computer-implemented method of characterizing a position and/or a movement of an intracardial structure of a heart relative to an intracardial probe, the method including: accessing a time series of impedance measurements made using at least one electrode of the intracardial probe positioned within a lumen of the heart; accessing a synchronizing indication including the timing of an event, wherein occurrence of the event is associated with a characteristic movement of the intracardial structure with a characteristic timing relative to the timing of the event; selecting, using the synchronizing indication and the association with the characteristic movement, impedance measurements from the time series measured at times concurrent with the characteristic movement of the intracardial structure; and characterizing the position and/or the movement of the intracardial structure based on a signal within the selected impedance measurements.

According to some embodiments of the present disclosure, the intracardial structure includes a portion of at least one leaflet of a heart valve, and the characteristic movement includes opening and/or closing of the heart valve.

According to some embodiments of the present disclosure, the characterization of the position of the intracardial structure includes an estimated distance between the at least one electrode and the intracardial structure.

According to some embodiments of the present disclosure, the characterization of the position of the intracardial structure includes an estimated direction of the intracardial structure relative to the at least one electrode.

According to some embodiments of the present disclosure, the characterization of the position and/or the movement of the intracardial structure includes an estimated amplitude of motion of the intracardial structure.

According to some embodiments of the present disclosure, the characterization of the position and/or the movement of the intracardial structure distinguishes a plurality of portions of the intracardial structure.

According to some embodiments of the present disclosure, the at least one electrode includes a plurality of electrodes; and the characterizing is further based on estimated relative positions of the plurality of electrodes.

According to some embodiments of the present disclosure, the estimated relative positions of the plurality of electrodes are substantially within a common plane.

According to some embodiments of the present disclosure, the at least one electrode includes an electrode which moved during measurement of the time series of impedance measurements; and the characterizing is further based on estimated movements of the moving electrode.

According to some embodiments of the present disclosure, the movement of the intracardial structure includes a periodic component.

According to some embodiments of the present disclosure, the synchronizing indication includes a feature of an electrophysiological signal.

According to some embodiments of the present disclosure, the electrophysiological signal includes an ECG signal.

According to some embodiments of the present disclosure, the feature includes at least a portion of an ECG signal's QRS waveform.

According to some embodiments of the present disclosure, the feature includes at least a portion of an ECG signal's P waveform.

According to some embodiments of the present disclosure, the synchronizing indication includes a feature of an audible sound signal.

According to some embodiments of the present disclosure, the synchronizing indication includes a feature of a pressure signal.

According to some embodiments of the present disclosure, the synchronizing indication includes a feature of a blood volume signal.

According to some embodiments of the present disclosure, the synchronizing indication includes a feature of an ultrasound signal.

According to some embodiments of the present disclosure, the signal within the selected impedance measurements includes impedance changes as a function of time due to the characteristic movement of the intracardial structure.

According to some embodiments of the present disclosure, the time series of impedance measurements includes at least one additional signal, additional to the signal within the selected impedance measurements used to characterize the position of the intracardial structure, and wherein the signal includes impedance changes as a function of time due to another body movement.

According to some embodiments of the present disclosure, the at least one additional signal is due to one or more of heart muscle contraction and respiratory movements.

According to some embodiments of the present disclosure, the characterizing includes subtraction of the at least one additional signal from the selected impedance measurements.

According to some embodiments of the present disclosure, the characterizing of the position and/or movement of the intracardial structure is based on an amplitude of the signal within the selected impedance measurements.

According to some embodiments of the present disclosure, the synchronizing indication indicates the time of occurrence of a movement occurring during measurement of the selected impedance measurements.

According to some embodiments of the present disclosure, the selecting includes identifying a peak or trough in the time series of impedance measurements at least partially coincident with the time of occurrence of the movement.

According to some embodiments of the present disclosure, the synchronizing indication indicates the time of occurrence of a movement occurring after measurement of the selected impedance measurements.

According to some embodiments of the present disclosure, the selecting includes identifying a peak or trough in the time series of impedance measurements offset within a predetermined time range from the time of occurrence of the movement.

According to some embodiments of the present disclosure, the time series of impedance measurements is made using a plurality of electrodes of the intracardial probe, as impedance between electrodes the plurality of electrodes.

According to some embodiments of the present disclosure, the method includes displaying the characterizing position and/or movement of the intracardial structure.

According to some embodiments of the present disclosure, the impedance measurements include injecting current to a first electrode of the intracardial probe at a first frequency and measuring voltage between the first electrode and a second electrode of the intracardial probe at the first frequency.

According to some embodiments of the present disclosure, the impedance measured is a self-impedance.

According to some embodiments of the present disclosure, the characterizing the position and/or movement of the intracardial structure includes estimating distance between an electrode of the intracardial probe and the intracardial structure.

According to an aspect of some embodiments of the present disclosure, there is provided a system including a processor, memory, and digital input/output facilities, and a program in the memory that instructs the processor to: access, from the memory and/or using the digital input/output facilities, a time series of impedance measurements made using at least one electrode of an intracardial probe positioned within a lumen of the heart; access, from the memory and/or using the digital input/output facilities, a synchronizing indication including the timing of an event, wherein occurrence of the event is associated with a characteristic movement of an intracardial structure with a characteristic timing relative to the timing of the event; select, using the synchronizing indication and the association with the characteristic movement, impedance measurements from the time series measured at times concurrent with the characteristic movement of the intracardial structure; characterize the position and/or the movement of the intracardial structure based on a signal within the selected impedance measurements; and provide the characterization, using the digital input/output facilities.

According to an aspect of some embodiments of the present disclosure, there is provided a computer-implemented method of identifying an intracardial structure of a heart, the method including: accessing a time series of impedance measurements made using at least one electrode of an intracardial probe positioned within a lumen of the heart; accessing a synchronizing indication including the timing of an event, wherein occurrence of the event is associated with a characteristic movement of the intracardial structure with a characteristic timing relative to the timing of the event; selecting, using the synchronizing indication and the association with the characteristic movement, impedance measurements from the time series measured at times concurrent with the characteristic movement of the intracardial structure; and identifying the intracardial structure, based on a signal within the selected impedance measurements.

According to an aspect of some embodiments of the present disclosure, there is provided a system including a processor, memory, and digital input/output facilities, and a program in the memory that instructs the processor to: access, from the memory and/or using the digital input/output facilities, a time series of impedance measurements made using at least one electrode of an intracardial probe positioned within a lumen of the heart; access, from the memory and/or using the digital input/output facilities, a synchronizing indication including the timing of an event, wherein occurrence of the event is associated with a characteristic movement of an intracardial structure with a characteristic timing relative to the timing of the event; select, using the synchronizing indication and the association with the characteristic movement, impedance measurements from the time series measured at times concurrent with the characteristic movement of the intracardial structure; identify the intracardial structure based on a signal within the selected impedance measurements; and provide the identification, using the digital input/output facilities.

According to an aspect of some embodiments of the present disclosure, there is provided a computer-implemented method of imaging an intracardial structure of a heart, the method including: accessing a time series of impedance measurements made using at least one electrode of an intracardial probe positioned within a lumen of the heart; accessing a synchronizing indication including the timing of an event, wherein occurrence of the event is associated with a characteristic movement of the intracardial structure with a characteristic timing relative to the timing of the event; selecting, using the synchronizing indication and the association with the characteristic movement, impedance measurements from the time series measured at times concurrent with the characteristic movement of the intracardial structure; and imaging the intracardial structure based on a signal within the selected impedance measurements.

According to an aspect of some embodiments of the present disclosure, there is provided a computer-implemented method of identifying an intracardial structure of a heart relative to an intracardial probe, the method including: accessing a time series of impedance measurements made using at least one electrode of the intracardial probe positioned within a lumen of the heart; accessing body surface electrocardiogram (BS-ECG) measurements; selecting, using the BS-ECG measurements, impedance measurements from the time series measured at times concurrent with the BS-ECG measurements; and identifying the intracardial structure based on a signal within the selected impedance measurements.

According to an aspect of some embodiments of the present disclosure, there is provided a computer-implemented method of characterizing a position and/or movement of an intracardial structure of a heart relative to an intracardial probe, the method including: accessing a time series of impedance measurements made using at least one electrode of the intracardial probe positioned within a lumen of the heart; accessing body surface electrocardiogram (BS-ECG) measurements; selecting, using the body surface electrocardiogram (BS-ECG) measurements, impedance measurements from the time series measured at times concurrent with the body surface electrocardiogram (BS-ECG) measurements; and characterizing the position and/or movement of the intracardial structure based on a signal within the selected impedance measurements.

According to an aspect of some embodiments of the present disclosure, there is provided a computer-implemented method of imaging an intracardial structure of a heart relative to an intracardial probe, the method including: accessing a time series of impedance measurements made using at least one electrode of the intracardial probe positioned within a lumen of the heart; accessing body surface electrocardiogram (BS-ECG) measurements; selecting, using the body surface electrocardiogram (BS-ECG) measurements, impedance measurements from the time series measured at times concurrent with the body surface electrocardiogram (BS-ECG) measurements; and imaging the intracardial structure based on a signal within the selected impedance measurements.

According to an aspect of some embodiments of the present disclosure, there is provided a method of impedance imaging including using the timing of events within ECG time series data to assist in generating a reconstruction of cardiac geometry from impedance measurement time series data.

According to an aspect of some embodiments of the present disclosure, there is provided a method of impedance imaging including using the timing of events within a first electrical measurement time series made using a first intracardiac electrode to assist in generating a reconstruction of cardiac geometry using a motion signal recorded in a second electrical measurement time series made using a second intracardiac electrode.

According to an aspect of some embodiments of the present disclosure, there is provided a method of identifying a heart wall of a patient, the method including: accessing impedance data collected using electrodes touching the heart wall during a heartbeat; comparing time development of the impedance data during the heart beat to reference data; and identifying the heart wall based on the comparison.

According to some embodiments of the present disclosure, the impedance data includes impedance values measured between two electrodes that touched the wall during the measurement.

According to some embodiments of the present disclosure, the impedance data includes impedance values synchronized with body surface ECG signals collected at the same time the impedance values were collected.

According to some embodiments of the present disclosure, the heart wall is a wall between a right atrium and a left atrium, or a wall between a right atrium and an aorta.

According to some embodiments of the present disclosure, comparing the time development includes comparing time derivative of the impedance data at one or more predetermined time points along the heartbeat.

According to some embodiments of the present disclosure, the one or more time points include at least one of the QRS complex and the T wave.

According to some embodiments of the present disclosure, the method further includes comparing average and/or variance of impedance values during a heartbeat, and wherein the identifying is further based on a comparison between the average and/or variance to corresponding reference data.

According to some embodiments of the present disclosure, the method further includes obtaining the impedance data from row impedance data including impedance data collected by electrodes that did not touch the wall during a heartbeat, identifying, in the row impedance data, data collected by electrodes that did not touch the wall during the heartbeat, and removing from the row impedance data values measured by electrodes that did not touch the wall during the heart beat to obtain the impedance data.

According to some embodiments of the present disclosure, the comparing and identifying is by a classifier based on supervised learning algorithm.

According to some embodiments of the present disclosure, identifying the heart wall consists of identifying if the heart wall is a predefined heart wall or any other heart wall.

According to some embodiments of the present disclosure, the predefined heart wall is the heart wall between the right and left atria.

According to an aspect of some embodiments of the present disclosure, there is provided an apparatus for identifying a heart wall of a patient, the apparatus including: a processor having access to one or more digital memories storing impedance data collected using electrodes touching the heart wall during at least one heartbeat and reference data, the processor being configured to compare time development of the impedance data during the at least one heartbeat to the reference data; identify the heart wall based on the comparison; and display an indication to what heart wall was identified.

According to some embodiments of the present disclosure, the reference impedance data is indicative to impedance values measured using electrodes touching each of the heart walls during at least 8010 heartbeats, and data indicative to ECG measurements made at the same time the impedance measurements were made.

According to some embodiments of the present disclosure, the heart walls comprise a wall between a right atrium and a left atrium, and a wall between a right atrium and an aorta.

According to some embodiments of the present disclosure, the reference data includes time derivative of the impedance data at one or more predetermined time points along the heartbeat, and the processor is configured to compare the one or more time derivatives to respective time derivatives in the reference data to identify the heart wall.

According to some embodiments of the present disclosure, the processor is further configured to extract from the accessed data time derivative of the impedance data at one or more predetermined time points along the heartbeat, and compare the extracted time derivative to respective time derivatives in the reference data to identify the heart wall.

According to some embodiments of the present disclosure, the one or more time points include at least one of the QRS complex and the T wave.

According to some embodiments of the present disclosure, the processor is configured to identify the heart wall based on a comparison between the time development and one or more of average and variance of impedance values measured during a heartbeat to corresponding reference average and/or variance.

According to some embodiments of the present disclosure, the apparatus is configured to apply a classifier based on supervised learning algorithm to compare the impedance data to the reference impedance data and identify the heart wall based on the comparison.

According to some embodiments of the present disclosure, the digitally stored impedance data includes impedance data collected from electrodes that did not touch the heart wall, and the processor is configured to extract from the digitally stored impedance data the impedance data collected using electrodes touching the heart wall.

According to some embodiments of the present disclosure, the processor is configured to classify the heart wall as belonging to one of a plurality of classes of heart walls.

According to some embodiments of the present disclosure, the plurality of classes includes one class that is a heart wall between the right and left atria, and one class that is a heart wall other than the heart wall between the left and right atria.

According to some embodiments of the present disclosure, the apparatus further includes: a first input for receiving readings from an intracardiac catheter; a second input for receiving readings from body surface ECG; and a processor configured to associate readings from the first and second inputs to generate the impedance data, and store the generated impedance data on the one or more digital memories.

According to an aspect of some embodiments of the present disclosure, there is provided a method of indicating locations identified to be leaflet locations in an image of a body part including an A/V plane, the method including: identifying a plane in the image as the A/V plane based on IEGM signals measured in the body part by one or more electrodes of an intra-body probe, each IEGM signal being associated with a respective location in the image; identifying each of a plurality of image points residing in the vicinity of the plane as a leaflet point or non-leaflet point based on impedance value associated with the respective image point; and displaying the image with locations corresponding to points identified as leaflet points being displayed differently than locations corresponding to points identified as non-leaflet points.

According to some embodiments of the present disclosure, identifying a plane as the A/V plane includes: accessing IEGM data including IEGM signals synchronized with ECG signals, wherein each IEGM signal is associated with a location in the image; and identifying, based on the IEGM data, ventricle locations in the image associated with IEGM signals, the synchronization of which to the ECG signal being indicative to touching a wall of a ventricle; identifying, based on the IEGM data, atrium locations in the image associated with IEGM signals, the synchronization of which to the ECG signal being indicative to touching a wall of an atrium; identifying as the A/V plane a plane separating ventricle points from atrium points.

According to some embodiments of the present disclosure, the plane separating the points is identified using a classifier, for example, a support vector machine (SVM) supervised learning model or a stochastic gradient decent optimization method.

According to some embodiments of the present disclosure, the location associated with the IEGM signal corresponds to a location in the body part, occupied by the one or more electrodes when the IEGM signal was measured.

According to some embodiments of the present disclosure, each impedance value associated with an image point is an impedance value measured using two electrodes of the intra-body probe, when at least one of the two electrodes was in a location in the body part, corresponding to the respective point in the image.

According to some embodiments of the present disclosure, displaying the image includes: accessing impedance data, associating impedance values with locations in the image and a phase in a heartbeat; and simultaneously displaying locations corresponding to points identified as leaflet points in different heartbeats during a common heartbeat phase.

According to some embodiments of the present disclosure, the method includes repeating the simultaneously displaying, so that consecutive displays correspond to consecutive heartbeat phases.

According to an aspect of some embodiments of the present disclosure, there is provided an apparatus configured to indicate leaflet locations in an image of a body part including an A/V plane, the apparatus including a memory, a processor, and a display, wherein the memory stores: the image of the body part; IEGM data, including a plurality of IEGM signals measured in the body part by one or more electrodes of an intra-body probe, each IEGM signal being associated with a respective location in the image; Impedance data, including, for a plurality of image points, a respective impedance value associated with the respective image point; instructions, that when executed by the processor cause the processor to: identify a plane in the image as the A/V plane based on the IEGM data; identify each of a plurality of image points residing in the vicinity of the plane as a leaflet point or non-leaflet point based on the impedance data; and control the display to display the image with locations corresponding to points identified as leaflet points being displayed differently than locations corresponding to points identified as non-leaflet points.

According to some embodiments of the present disclosure, each IEGM signal in the IEGM data is further associated with a respective heartbeat phase, during which the IEGM signal was measured; and the instructions cause the processor to: identify, based on the IEGM data, ventricle locations in the image associated with IEGM signals, the heartbeat phase associated therewith being indicative to touching a wall of a ventricle; identify, based on the IEGM data, atrium locations in the image associated with IEGM signals, the heartbeat phase associated therewith being indicative to touching a wall of an atrium; and identify a plane separating ventricle points from atrium points as the A/V plane.

According to some embodiments of the present disclosure, the instructions cause the processor to separate ventricle points from atrium point using a classifier, for example, a support vector machine (SVM) supervised learning model or a stochastic gradient decent optimization method.

According to some embodiments of the present disclosure, the location in the image associated with the IEGM signal corresponds to a location in the body part, occupied by the at least one electrode when the IEGM signal was measured.

According to some embodiments of the present disclosure, each impedance value associated with an image point is an impedance value measured by using two electrodes of the intra-body probe, when at least one of the two electrodes was in a location in the body part, corresponding to the respective point in the image.

According to some embodiments of the present disclosure, the impedance data further includes a respective heartbeat phase associated with each of the plurality of image points, and the instructions cause the processor to: access the impedance data; and cause the display to simultaneously display locations corresponding to points identified as leaflet points in different heartbeats during a common heartbeat phase.

According to some embodiments of the present disclosure, the instructions cause the processor to repeat causing the display to simultaneously display the locations, so that consecutive displays correspond to consecutive heartbeat phases.

According to an aspect of some embodiments of the present disclosure, there is provided a method of mapping a vascular lumen extending alongside a body cavity, the method including: moving a first probe within the body cavity while measuring signals from a plurality of electrical fields; moving a second probe through the vascular lumen while measuring signals from the plurality of electrical fields; reconstructing a shape of the body cavity and the vascular lumen using the measurements of the first probe and the second probe; and identifying vascular lumen positions within the reconstructed shape corresponding to measurement positions of the second probe.

According to some embodiments of the present disclosure, the method includes displaying the reconstructed shape with an indication of the vascular lumen positions.

According to some embodiments of the present disclosure, the body cavity includes one or more heart chambers, and the vascular lumen includes coronary vasculature.

According to some embodiments of the present disclosure, the method includes tracking the position of a potentially traumatizing device as it moves through the body cavity, and estimating the distance of the potentially traumatizing device to the vascular lumen positions. According to some embodiments of the present disclosure, the potentially traumatizing device is a fastener for an implantable device.

According to some embodiments of the present disclosure, the implantable device is an annuloplasty device.

According to some embodiments of the present disclosure, the method includes providing an indication that the potentially traumatizing device is within a potentially hazardous proximity to the vascular lumen.

According to an aspect of some embodiments of the present disclosure, there is provided a method of locating a heart valve annulus along an atrioventricular axis, the method including: measuring intracardiac electrophysiological signal waveforms from probe positions extending between an atrial side and a ventricular side of a heart valve annulus, including, for each side, a respective plurality of probe positions; determining relative spatial locations of the probe positions within the heart; and identifying, based on the signal waveform measurements, a position and orientation of a region between the atrial side and the ventricular side, the region being positioned at the heart valve annulus along the atrioventricular axis, and oriented to include opposite circumferential sides of the heart valve annulus.

According to some embodiments of the present disclosure, the spatial locations correspond to locations within a 3-D model of the heart.

According to some embodiments of the present disclosure, the method includes displaying the 3-D model of the heart marked with the identified spatial locations at the position of the heart valve annulus along the atrioventricular axis.

According to some embodiments of the present disclosure, the method includes identifying at least one of the spatial locations as being at the position of an atrium along the atrioventricular axis, based on the signal waveform measured at the at least one of the spatial locations.

According to some embodiments of the present disclosure, the method includes displaying a 3-D model of the heart marked with the identified at least one of the spatial locations at the position of the atrium along the atrioventricular axis.

According to some embodiments of the present disclosure, the method includes identifying at least one of the spatial locations as being at the position of a ventricle along the atrioventricular axis, based on the signal waveform measured at the at least one of the spatial locations.

According to some embodiments of the present disclosure, the method includes displaying a 3-D model of the heart marked with the identified at least one of the spatial locations at the position of the ventricle along the atrioventricular axis.

According to some embodiments of the present disclosure, the identification of position along the atrioventricular axis includes identification of relative amplitudes of an atrially-generated electrophysiological signal, and a ventricularly-generated electrophysiological signal.

According to some embodiments of the present disclosure, the atrially generated signal includes a P wave of an electrocardiogram.

According to some embodiments of the present disclosure, the ventricularly generated signal includes a QRS complex of an electrocardiogram.

According to some embodiments of the present disclosure, the identifying includes interpolating between atrial-side and ventricular side measurements of the electrophysiological signal waveforms to identify one or more intermediate positions at the heart valve annulus. According to some embodiments of the present disclosure, the identifying identifies a planar region intersecting an entire circumference of the valve annulus.

According to some embodiments of the present disclosure, the identifying identifies a non-planar region intersecting an entire circumference of the valve annulus.

According to some embodiments of the present disclosure, the non-planar region is saddle-shaped due to a geometric deformity of the valve annulus.

According to an aspect of some embodiments of the present disclosure, there is provided a method of automatically locating a hinge of a heart valve annulus, the method including: accessing a 3-D model including the heart valve annulus and at least a portion of the heart valve leaflets; determining elevation angles of surface orientation relative to the valve annulus along a plurality of radii of the valve annulus; and identifying, along the plurality of radii, respective positions that are portions of the hinge, based on elevation angle and/or changes in elevation angle along its respective radius.

According to some embodiments of the present disclosure, the location of the heart valve annulus surface used in determining the elevation angles is at least partially determined based on electrophysiological measurements measured from positions along an atrioventricular axis extending through the heart valve annulus.

According to an aspect of some embodiments of the present disclosure, there is provided a method of identifying extents of fibrous tissue within a heart valve annulus, the method including: measuring an electrical signal indicating impedance changes from probe positions in proximity to tissue extending from connective tissue of a valve annulus to myocardial tissue surrounding the valve annulus; determining relative spatial locations of the probe positions within the heart; and identifying a plurality of the spatial locations as including connective tissue of the valve annulus, based on the measured electrical signal.

According to some embodiments of the present disclosure, the method includes displaying an indication of the identification.

According to some embodiments of the present disclosure, the method includes measuring a time course of changes in the measured electrical signal due to motion of tissue, and identifying a plurality of the spatial locations as including connective tissue of the valve annulus, distinct from leaflets of the valve, based on the measured time course of changes in the electrical signal.

According to an aspect of some embodiments of the present disclosure, there is provided a method of detecting valve leaflets, the method including: measuring electrical signals indicative of impedance changes from one or more electrodes located near a cardiac valve; and determining if the electrode is near a leaflet of the cardiac valve, based on a characteristic of electrical signal changes during a single heartbeat cycle.

According to an aspect of some embodiments of the present disclosure, there is provided a method of identifying hinge boundary between a heart valve annulus and heart valve leaflets, the method including: measuring, at a plurality of probe positions in proximity to one or both of the heart valve annulus and the heart valve leaflets, a time course of changes in an electrical signal indicative of impedance changes due to motion of tissue; determining relative spatial locations of the probe positions within the heart; analyzing motions detected at the plurality of probe positions as characteristic of motions of the valve annulus or of the valve leaflets; and identifying as valve hinge positions spatial locations between valve annulus locations and valve leaflet locations.

According to some embodiments of the present disclosure, the method includes presenting an indication of the identifications of valve hinge positions.

According to some embodiments of the present disclosure, motion-induced electrical signal changes characteristic of the valve leaflets are indicative of a doubled cycle of increasing and decreasing impedance during a single heartbeat cycle.

According to some embodiments of the present disclosure, motion-induced electrical signal changes characteristic of the valve annulus are indicative of a single cycle of increasing and decreasing impedance during a single heartbeat cycle.

According to some embodiments of the present disclosure, the location of the heart valve annulus along the atrioventricular axis is determined based on electrophysiological measurements measured from positions along the atrioventricular axis; and the probe positions selected according to the determined location of the heart valve annulus.

According to an aspect of some embodiments of the present disclosure, there is provided a method of locating a structure of the electrical conduction system of the heart, the method including: measuring intracardiac electrophysiological signal waveforms from intracardial probe positions; determining spatial locations of the intracardial probe positions within the heart; and identifying at least one of the spatial locations as being at the position of the structure, based on the signal waveform measured at the at least one of the spatial locations.

According to some embodiments of the present disclosure, the structure includes a bundle of His, and the signal waveform is characteristic of a latency to waveform arrival at the bundle of His.

According to some embodiments of the present disclosure, the structure includes an AV node, and the signal waveform is characteristic of a latency to waveform arrival at the AV node.

According to an aspect of some embodiments of the present disclosure, there is provided a method of planning implantation of an annuloplasty device to a heart valve, the method including: locating a circumferentially extending portion of a hinge of the heart valve; locating a circumferentially extending portion of a coronary artery; identifying a pathway extending along and between the locations of the two circumferentially extending portions; and providing the identified pathway as a target location for implantation of the annuloplasty device.

According to some embodiments of the present disclosure, the circumferentially extending portion of the hinge is identified automatically, based on measurements of one or more of a geometry of the heart valve, dielectric properties of the heart valve, and electrophysiological signals measured near the heart valve.

According to some embodiments of the present disclosure, the circumferentially extending portion of the hinge is identified by manual selection.

According to some embodiments of the present disclosure, the portion of the coronary artery is identified using electrical field imaging.

According to some embodiments of the present disclosure, the electrical field imaging includes using probes separated by a tissue barrier to map electrical field voltages on either side of the barrier.

According to some embodiments of the present disclosure, the electrical field imaging includes: using a probe moving within a lumen including a valve annulus of the heart valve; and identifying positions at which the probe senses a signal transmitted from within the coronary artery, at an amplitude indicative of close proximity to the coronary artery.

According to some embodiments of the present disclosure, the method includes locating a structure of the heart electrical conduction system; and including adjusting the pathway to remain at least a predetermined distance away from the located structure.

According to an aspect of some embodiments of the present disclosure, there is provided a method of monitoring the implantation of a fastener for an annuloplasty device into a valve annulus, the method including: measuring an electrical signal indicative of impedance using the fastener as an electrode, while the fastener is being brought to an implantation position; providing an indication of fastener position, based on a change in the measured electrical signal.

According to some embodiments of the present disclosure, the fastener is a screw.

According to some embodiments of the present disclosure, the indication indicates fastener contact with tissue of the valve annulus.

According to some embodiments of the present disclosure, the method includes inserting an electrode to a coronary artery; wherein the indication warns of fastener penetration of the coronary artery.

According to some embodiments of the present disclosure, the method includes inserting an electrode to a coronary artery; wherein the impedance is between the fastener and the inserted electrode; and the indication is an indication of fastener proximity to the coronary artery.

According to some embodiments of the present disclosure, the indication indicates a depth of fastener penetration into the valve annulus.

According to some embodiments of the present disclosure, the indication of fastener position is also based on the determination that the fastener is located at a valvular position along the axis.

According to an aspect of some embodiments of the present disclosure, there is provided a method of monitoring the implantation of a fastener for an annuloplasty device into a valve annulus, the method including: receiving a specification of implantation positions within a heart posing a risk of damage to a right coronary artery; tracking entry of a fastener to one of the specified implantation positions; and providing, based on the tracked entry, an indication that the fastener is positioned where there is a risk of damage to the right coronary artery.

According to an aspect of some embodiments of the present disclosure, there is provided a method of determining the distance of positions in a first blood-filled lumen from a transmitter probe located in a second blood-filled lumen, the method including: placing a transmitter probe in the second blood-filled lumen; transmitting a signal from the transmitter probe; recording the signal using a sensor positioned on a probe positioned in the first blood-filled lumen; wherein the first and second blood-filled lumens are separated from each other across a barrier of solid tissue; and estimating a distance between the sensor and the transmitter probe, based on an amplitude of the recorded signal.

According to some embodiments of the present disclosure, the transmitted signal includes one or more of an electrical signal, a magnetic signal, and an acoustic signal.

According to some embodiments of the present disclosure, the first blood-filled lumen is a heart chamber, and the second blood-filled lumen is a cardiac artery.

According to some embodiments of the present disclosure, the transmitter probe transmits from a plurality of distinguishable segments along the transmitter probe.

According to some embodiments of the present disclosure, the distance estimation is adjusted according to the segment from which the signal is received.

According to some embodiments of the present disclosure, the distance estimation uses just one of the plurality of distinguishable segments.

According to some embodiments of the present disclosure, the method includes providing a proximity warning, based on the estimated distance.

According to an aspect of some embodiments of the present disclosure, there is provided a system configured to map a vascular lumen extending alongside a body cavity, the system including: a processor, memory storing instructions, and display; wherein the processor is configured to receive respective inputs from: a first probe moving within the body cavity while measuring signals from a plurality of electrical fields, and a second probe through the vascular lumen while measuring signals from the plurality of electrical fields; and wherein the processor operates according to the instructions to: reconstruct a shape of the body cavity and the vascular lumen using the measurements of the first probe and the second probe, and identify vascular lumen positions within the reconstructed shape corresponding to measurement positions of the second probe, and present an image the reconstructed shape on the display with an indication of the vascular lumen position.

According to some embodiments of the present disclosure, the processor further operates to track, based on position measurements received, the position of a potentially traumatizing device as it moves through the body cavity, and estimate the distance of the potentially traumatizing device to the vascular lumen positions.

According to some embodiments of the present disclosure, the potentially traumatizing device is a fastener for an implantable device.

According to some embodiments of the present disclosure, the implantable device is an annuloplasty device.

According to some embodiments of the present disclosure, the processor presents an indication that the potentially traumatizing device is within a potentially hazardous proximity to the vascular lumen.

According to an aspect of some embodiments of the present disclosure, there is provided a system configured to locate a heart valve annulus along an atrioventricular axis, the system including: a processor, memory storing instructions, and display; wherein the processor is configured to receive intracardiac electrophysiological signal waveforms measured from: probe positions extending between an atrial side and a ventricular side of a heart valve annulus, including, for each side, a respective plurality of probe positions; wherein the processor operates according to the instructions to: determine relative spatial locations of the probe positions within the heart, identify, based on the signal waveform measurements, a position and orientation of a region between the atrial side and the ventricular side, the region being positioned at the heart valve annulus along the atrioventricular axis, and oriented to include opposite circumferential sides of the heart valve annulus, and produce a model of the heart.

According to some embodiments of the present disclosure, the spatial locations correspond to locations within a 3-D model of the heart.

According to some embodiments of the present disclosure, the processor further operates to present on the display the 3-D model of the heart marked with the identified spatial locations at the position of the heart valve annulus along the atrioventricular axis.

According to some embodiments of the present disclosure, the processor operates to identify at least one of the spatial locations as being at the position of an atrium along the atrioventricular axis, based on the signal waveform measured at the at least one of the spatial locations.

According to some embodiments of the present disclosure, the processor operates to present on the display a 3-D model of the heart marked with the identified at least one of the spatial locations at the position of the atrium along the atrioventricular axis.

According to some embodiments of the present disclosure, the processor operates to identify at least one of the spatial locations as being at the position of a ventricle along the atrioventricular axis, based on the signal waveform measured at the at least one of the spatial locations.

According to some embodiments of the present disclosure, the processor operates to present on the display a 3-D model of the heart marked with the identified at least one of the spatial locations at the position of the ventricle along the atrioventricular axis.

According to some embodiments of the present disclosure, the processor identifies position along the atrioventricular axis by identifying relative amplitudes of an atrially-generated electrophysiological signal, and a ventricularly-generated electrophysiological signal.

According to some embodiments of the present disclosure, the atrially generated signal includes a P wave of an electrocardiogram.

According to some embodiments of the present disclosure, the ventricularly generated signal includes a QRS complex of an electrocardiogram.

According to some embodiments of the present disclosure, the processor identifies position along the atrioventricular axis by interpolating between atrial-side and ventricular side measurements of the electrophysiological signal waveforms to identify one or more intermediate positions at the heart valve annulus.

According to some embodiments of the present disclosure, the processor identifies position along the atrioventricular axis by identifying a planar region intersecting an entire circumference of the valve annulus.

According to some embodiments of the present disclosure, the processor identifies position along the atrioventricular axis by identifying a non-planar region intersecting an entire circumference of the valve annulus.

According to some embodiments of the present disclosure, the non-planar region is saddle-shaped due to a geometric deformity of the valve annulus.

According to an aspect of some embodiments of the present disclosure, there is provided a system configured to automatically locate a hinge of a heart valve annulus, the system including: a processor and memory storing instructions; wherein the processor is configured to access a 3-D model including: the heart valve annulus, and at least a portion of the heart valve leaflets; and wherein the processor operates according to the instructions to: determine elevation angles of surface orientation relative to the valve annulus along a plurality of radii of the valve annulus; and identify, along the plurality of radii, respective positions that are portions of the hinge, based on elevation angle and/or changes in elevation angle along its respective radius.

According to some embodiments of the present disclosure, the location of the heart valve annulus surface used in determining the elevation angles is at least partially determined based on electrophysiological measurements measured from positions along an atrioventricular axis extending through the heart valve annulus.

According to an aspect of some embodiments of the present disclosure, there is provided a system configured to identifying extents of fibrous tissue within a heart valve annulus, the system including: a processor, memory storing instructions, and display; wherein the processor is configured to receive: measurements of an electrical signal indicating impedance changes, the measurements being obtained from probe positions in proximity to tissue extending from connective tissue of a valve annulus to myocardial tissue surrounding the valve annulus, and relative spatial locations of the probe positions within the heart; and wherein the processor operates according to the instructions to: identify a plurality of the spatial locations as including connective tissue of the valve annulus, based on the measured electrical signal, and present an indication of the identification on the display.

According to some embodiments of the present disclosure, the system includes measuring a time course of changes in the measured electrical signal due to motion of tissue, and identifying a plurality of the spatial locations as including connective tissue of the valve annulus, distinct from leaflets of the valve, based on the measured time course of changes in the electrical signal.

According to an aspect of some embodiments of the present disclosure, there is provided a system configured to detect valve leaflets, the system including: a processor and memory storing instructions; wherein the processor is configured to receive measurements of electrical signals indicative of impedance changes from one or more electrodes located near a cardiac valve; and wherein the processor operates according to the instructions to: determine if the electrode is near a leaflet of the cardiac valve, based on a characteristic of electrical signal changes during a single heartbeat cycle.

According to an aspect of some embodiments of the present disclosure, there is provided a system configured to identify hinge boundary between a heart valve annulus and heart valve leaflets, the system including: a processor, memory storing instructions, and display; wherein the processor is configured to receive respective inputs measuring, at a plurality of probe positions in proximity to one or both of the heart valve annulus and the heart valve leaflets, a time course of changes in an electrical signal indicative of impedance changes due to motion of tissue; and wherein the processor operates according to the instructions to: determine relative spatial locations of the probe positions within the heart, determine motions detected at the plurality of probe positions as characteristic of motions of the valve annulus or of the valve leaflets, determine, as being valve hinge positions, spatial locations between valve annulus locations and valve leaflet locations, and present, using the display, an indication of the identifications of valve hinge positions.

According to some embodiments of the present disclosure, the processor identifies motion-induced electrical signal changes characteristic of the valve leaflets based on a doubled cycle of increasing and decreasing impedance during a single heartbeat cycle.

According to some embodiments of the present disclosure, the location of the heart valve annulus along the atrioventricular axis is determined based on electrophysiological measurements measured from positions along the atrioventricular axis; and the probe positions selected according to the determined location of the heart valve annulus.

According to an aspect of some embodiments of the present disclosure, there is provided a system configured to locate a structure of the electrical conduction system of the heart, the system including: a processor and memory storing instructions; wherein the processor is configured to receive respective measurements of intracardiac electrophysiological signal waveforms made at intracardial probe positions; and wherein the processor operates according to the instructions to: determine spatial locations of the intracardial probe positions within the heart; and identify at least one of the spatial locations as being at the position of the structure, based on the signal waveform measured at the at least one of the spatial locations.

According to some embodiments of the present disclosure, the structure includes a bundle of His, and the signal waveform is characteristic of a latency to waveform arrival at the bundle of His.

According to some embodiments of the present disclosure, the structure includes an AV node, and the signal waveform is characteristic of a latency to waveform arrival at the AV node.

According to an aspect of some embodiments of the present disclosure, there is provided a system configured to plan implantation of an annuloplasty device to a heart valve, the system including: a processor and memory storing instructions; wherein the processor is configured to receive inputs defining: a circumferentially extending portion of a hinge of the heart valve; a circumferentially extending portion of a coronary artery; and wherein the processor operates according to the instructions to: define a pathway extending along and between the locations of the two circumferentially extending portions; and provide the identified pathway as a target location for implantation of the annuloplasty device.

According to some embodiments of the present disclosure, the circumferentially extending portion of the hinge is identified automatically, based on measurements of one or more of a geometry of the heart valve, dielectric properties of the heart valve, and electrophysiological signals measured near the heart valve.

According to some embodiments of the present disclosure, the circumferentially extending portion of the hinge is identified by manual selection.

According to some embodiments of the present disclosure, the portion of the coronary artery is identified using electrical field imaging.

According to some embodiments of the present disclosure, the electrical field imaging includes the use of measurement from probes separated by a tissue barrier to map electrical field voltages on either side of the barrier.

According to some embodiments of the present disclosure, the electrical field imaging includes: use of a probe moving within a lumen including a valve annulus of the heart valve; and identification of positions at which the probe senses a signal transmitted from within the coronary artery, at an amplitude indicative of close proximity to the coronary artery.

According to some embodiments of the present disclosure, the processor is instructed to receive a location of a structure of the heart electrical conduction system; and adjust the pathway to remain at least a predetermined distance away from the located structure.

According to an aspect of some embodiments of the present disclosure, there is provided a system configured to monitor the implantation of a fastener for an annuloplasty device into a valve annulus, the system including: a processor, memory storing instructions, and display; wherein the processor is configured to receive measurements of an electrical signal indicative of impedance using the fastener as an electrode, while the fastener is being brought to an implantation position; and wherein the processor operates according to the instructions to present to the display an indication of fastener position, based on a change in the measured electrical signal.

According to some embodiments of the present disclosure, the fastener is a screw.

According to some embodiments of the present disclosure, the indication indicates fastener contact with tissue of the valve annulus.

According to some embodiments of the present disclosure, the indication warns of fastener penetration of the coronary artery.

According to some embodiments of the present disclosure, measurements are of impedance between the fastener and an electrode inserted to the coronary artery; and the indication is an indication of fastener proximity to the coronary artery.

According to some embodiments of the present disclosure, the indication indicates a depth of fastener penetration into the valve annulus.

According to some embodiments of the present disclosure, the processor receives an estimated position of the fastener along an axis between an atrium and a ventricle; and the indication of fastener position is also based on the determination that the fastener is located at a valvular position along the axis.

According to an aspect of some embodiments of the present disclosure, there is provided a system configured to monitor the implantation of a fastener for an annuloplasty device into a valve annulus, the system including: a processor and memory storing instructions; wherein the processor is configured to receive: a specification of implantation positions within a heart posing a risk of damage to a right coronary artery, and position data indication positions of a fastener moving within the heart; wherein the processor operates according to the instructions to: track entry of the fastener to one of the specified implantation positions; and provide, based on the tracked entry, an indication that the fastener is positioned where there is a risk of damage to the right coronary artery.

According to an aspect of some embodiments of the present disclosure, there is provided a system configured to determine the distance of positions in a first blood-filled lumen from a transmitter probe located in a second blood-filled lumen, the system including: a processor, memory storing instructions, and display; wherein the processor is configured to receive a signal: sensed by a sensor positioned on a probe positioned in the first blood-filled lumen, and transmitted to the sensor from a transmitter probe in the second blood-filled lumen; wherein the processor operates according to the instructions to estimate a distance between the sensor and the transmitter probe, based on an amplitude of the received signal; and wherein the first and second blood-filled lumens are separated from each other across a barrier of solid tissue.

According to some embodiments of the present disclosure, the transmitted signal includes one or more of an electrical signal, a magnetic signal, and an acoustic signal.

According to some embodiments of the present disclosure, the first blood-filled lumen is a heart chamber, and the second blood-filled lumen is a cardiac artery.

According to some embodiments of the present disclosure, the transmitter probe transmits from a plurality of distinguishable segments along the transmitter probe.

According to some embodiments of the present disclosure, the distance estimation is adjusted according to the segment from which the signal is received.

According to some embodiments of the present disclosure, the distance estimation uses just one of the plurality of distinguishable segments.

According to some embodiments of the present disclosure, the processor presents, using the display, a proximity warning, based on the estimated distance.

According to an aspect of some embodiments of the present disclosure, there is provided a method of monitoring a structural heart disease intervention including introduction of a device into a heart chamber, the method including: accessing a structural representation of a portion of a heart; accessing electrophysiological measurements indicating electrical activity of tissue of the heart; associating the electrophysiological measurements to locations in the structural representation of the portion of the heart; presenting an image of the structural representation of the portion of the heart, together with indications of values of the electrophysiological measurements at their associated locations.

According to some embodiments of the present disclosure, the structural representation is a three-dimensional structural representation.

According to some embodiments of the present disclosure, the electrophysiological measurements comprise electrophysiological measurements recorded using the device.

According to some embodiments of the present disclosure, the method includes estimating a position of the device, using the electrophysiological measurements recorded using the device.

According to some embodiments of the present disclosure, the presented indications of the values of the electrophysiological measurements comprise identifications of different tissue structures associated with the electrophysiological measurement.

According to some embodiments of the present disclosure, the method comprises presenting an indication of a location to be avoided for attachment of the device, wherein the avoided location is determined based on the electrophysiological measurements.

According to some embodiments of the present disclosure, the avoided location is determined based on electrophysiological measurements at the location corresponding to electrophysiological characteristics of a bundle of His.

According to some embodiments of the present disclosure, the avoided location is determined based on electrophysiological measurements at the location corresponding to electrophysiological characteristics of an AV node.

According to some embodiments of the present disclosure, the device is an implantable annuloplasty device.

According to an aspect of some embodiments of the present disclosure, there is provided a method of guiding a structural heart disease intervention, including: accessing a structural representation of a heart; accessing electrophysiological measurements indicating electrical activity of tissue of the heart; associating the electrophysiological measurements to locations in the structural representation of the heart corresponding to locations at which the measurements were recorded; selecting a location for attachment of a device configured to provide structural heart disease intervention, based on the structural representation, the electrophysiological measurements, and their locations in the structural representation; and presenting an image of the structural representation of the heart wherein the selected location is marked.

According to an aspect of some embodiments of the present disclosure, there is provided a method of verifying a structural heart disease intervention, including: accessing a structural representation of a heart; accessing electrophysiological measurements obtained from locations within the heart before and after implantation to the heart of a device configured to provide a structural heart disease intervention; associating the electrophysiological measurements to locations in the structural representation of the heart corresponding to locations at which the measurements were recorded; and comparing electrophysiological activity before and after implantation of the device in at least one location of the heart to check for impairment of electrophysiological activity at the at least one location.

According to an aspect of some embodiments of the present disclosure, there is provided a method of guiding a structural heart disease intervention, including: accessing electrophysiological (EP) measurements of the heart measured from a specified location; and guiding the structural heart disease intervention based on the accessed EP measurements.

According to some embodiments of the present disclosure, guiding the structural heart diseases intervention includes indicating on an image of a portion of the heart a current location of an implant for use in the intervention, the specified location, and the accessed EP measurements.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a schematic flowchart of a method of guiding and monitoring implantation of a tricuspid heart valve annuloplasty device using a multimodal measurement approach, according to some embodiments of the present disclosure;

FIGS. 2A-2G schematically illustrate selected phases in the implantation of annuloplasty device, together with examples of auxiliary tools used to guide and/or monitor the implantation, according to some embodiments of the present disclosure.

FIG. 3A schematically illustrates implantation of an annuloplasty device for treatment of regurgitation in a mitral valve, according to some embodiments of the present disclosure.

FIG. 3B is a schematic flowchart of a method of guiding and monitoring implantation of a mitral heart valve annuloplasty device, according to some embodiments of the present disclosure.

FIG. 4A schematically represents an overhead view (looking from a right atrium toward a right ventricle) of a tricuspid valve, according to some embodiments of the present disclosure;

FIGS. 4B-4C schematically illustrate examples of displays used in device implantation planning and/or in performing device implantation, according to some embodiments of the present disclosure;

FIG. 5 schematically represents time traces of respiration (trace), and body surface ECG (trace), according to some embodiments of the present disclosure;

FIG. 6 schematically represents an annuloplasty device, according to some embodiments of the present disclosure;

FIG. 7A schematically illustrate a method of identifying valve hinge locations, according to some embodiments of the present disclosure;

FIG. 7B schematically illustrates a method of using time-frequency decomposition to distinguish components of heart structure as belonging to different structures, according to some embodiments of the present disclosure;

FIG. 8 schematically represents detection of wall contacts, according to some embodiments of the present disclosure; and

FIG. 9 is schematic diagram of a system for monitoring and/or guiding annuloplasty device implantation, according to some embodiments of the present disclosure.

FIG. 10A schematically illustrates coronary artery proximity and penetration by a device fastener, according to some embodiments of the present disclosure;

FIG. 10B schematically represents features of coupling measurements potentially useful to detect changes of coronary artery proximity and penetration by a fastener, according to some embodiments of the present disclosure.

FIGS. 11A-11D show four graphs exemplifying reference impedance data, according to some embodiments of the invention;

FIG. 12 shows reference impedance data and preferable point for measuring slopes thereof, according to some embodiments of the invention;

FIGS. 13-14 illustrate relative timings of body surface ECG events and intracardial impedance measurements indicative of movements of the right atrioventricular valve (tricuspid valve), according to some embodiments of the present disclosure;

FIG. 15 is a flowchart schematically outlining a method of selecting relevant impedance measurements from an impedance measurement time series, according to some embodiments of the present disclosure;

FIG. 16 is a diagram schematically outlining various embodiment implementations which convert impedance measurements from an impedance measurement time series into a estimates characterizing the position of a particular tissue structure, according to some embodiments of the present disclosure;

FIG. 17 is a schematic representation of a system for measuring the position of a moving intracardiac tissue structure, according to some embodiments of the present disclosure;

FIG. 18 schematically illustrates another method of estimating the position of the plane of the valve annulus (the AV plane), according to some embodiments of the present disclosure; and

FIG. 19 is a schematic flowchart of a method of mapping valve leaflets, according to some embodiments of the present disclosure.

### DESCRIPTION OF SPECIFIC EMBODIMENTS OF THE INVENTION

The present invention, in some embodiments thereof, relates to the field of navigation within body cavities by intrabody devices, and more particularly, to guidance of the placement of intrabody devices, optionally including implantable devices.

### Overview

An aspect of some embodiments of the present disclosure relates to the integration of multimodal measurements of an intrabody environment of a medical procedure—including measurements of structure and of events taking place therein—into a compound model which unifies them. The compound model may be used, in some embodiments, to guide and/or monitor the procedure. More particularly, in some embodiments, multimodal measurement comprises the integration of electrophysiological measurements with impedance measurements, and optionally also with spatial positioning measurements, to characterize internal structures of body lumens. In some embodiments, the body lumens are body lumens of the heart.

In some embodiments, structural characterization is performed within the overall context of an intervention to treat structural heart disease, for example annuloplasty, manipulation of the valve leaflets, closure of the left atrial appendage, valve replacement, or another procedure. The measurements may be performed for the guidance and monitoring of such procedures.

The term “multimodal measurement” refers to the use of measurements of a plurality of different types to characterize the intrabody environment of the procedure and/or activities taking place within it. The prefix “multi-” should be understood to apply to the overall approach, which is capable of integrating measurements made using several different approaches into a compound model of the intrabody environment. Any individual embodiment of the present disclosure optionally uses a particular set of a plurality of measurement approaches.

Embodiments of the present disclosure may be understood as establishing a “scaffolding” that is built (e.g., based on earlier measurements in a procedure, or inputs from pre-procedure data sources) to provide a basic model of a procedure's environment. Further measurements continuously provide further detail to and/or update that model, as they are associated to their appropriate places within the compound model. The compound model develops over time as a result. The association of new measurements is performed in a manner that supports guiding and/or monitoring a procedure in real time.

The scaffolding of the basic model is optionally based on a primary measurement modality (for example, imaging by electrical field measurements and/or ultrasound intra-cardiac echocardiography, ICE, MRI, and/or CT), or built up by the coordinated use of a plurality of measurement methods (and thus may be “compound” from the beginning). Measurements may be made using devices auxiliary to the annuloplasty device (e.g., imaging probes placed within the heart lumen or at other locations). Optionally, the annuloplasty device itself is used as a measurement device; for example, conductive elements of the device such as control wires and/or fasteners are configured as electrodes, and/or electrodes are attached to the device and/or positioned where they have a known spatial relationship to the device (e.g., on a sheath of a delivery catheter of the device).

Locations within the compound model are optionally described in terms of spatial coordinates (spatial positions) and/or distances; and/or in terms of non-spatial metrics which characterize a measurement, such as its signal phase, amplitude, and/or eigenvalue of one or more eigenvector components of the measurement (e.g., as determined by a method of mathematical decomposition). In some embodiments, a compound model includes both spatial and non-spatial representations. For example, electrophysiological measurements may be used to guide a procedure by the assessment of “similarity (of the measurement) to a target”, while “spatial distance to a target” may be provided in coordination with the similarity assessment, e.g., to confirm and/or refine it. In some embodiments, spatial positions are provided by an imaging method, for example, a reconstruction-type imaging method using samples taken from many points within a structure to reconstruct an image of the structure), or an imaging method based on probing radiant energy such as X-ray imaging or echocardiography, or another imaging method.

Embodiments of the present disclosure describe multimodal measurement-based solutions for problems which arise during the course of structural heart disease interventions, including problems associated with:
locating, identifying, characterizing and/or confirming one or more regions traversed by a catheter as it navigates to a treatment site;
locating, identifying, and/or characterizing a region targeted for implantation of a structural heart disease treatment device;
planning and/or actual implantation of the device which avoids damage to sensitive heart areas; and/or
verification of attachment of the device to the heart.

In some embodiments, multimodal measurement-based compound models combine electrophysiological measurements of heart activity with detailed positional (including detailed structural shape) information. In some embodiments, intracardiac measurements of endogenous electrical activity are localized in space by coordinating them closely with locations (e.g., the “scaffolding”) defined by the compound model. Optionally, the locations are themselves further characterized according to their suitability as sites for intervention, e.g., based on a determination that they comprise fibrous tissue of the valve annulus. The electrophysiology reveals, in some embodiments, locations the implantation should avoid (e.g., because certain electrically active tissue in the locations to be avoided is particularly vulnerable to mechanical damage). This information is optionally used to exclude device attachment at otherwise (e.g., mechanically) suitable attachment sites.

In some embodiments, locations characterized by electrophysiology are used to establish a frame of reference (e.g., a valve plane) which assists in the interpretation and/or gathering of measurements made using yet another measurement modality (e.g., impedance measurements). Optionally or additionally, the frame of reference is temporal—for example, ECG measurements made concurrently with impedance measurements are optionally used to “time lock” the measurement to a certain phase of the heartbeat cycle. This can assist in interpreting time-varying impedance measurements by limiting the range of interpretations on what nearby structure is moving to generate the time-varying impedance. This may help characterize, e.g., movements of heart valve leaflets and/or movements of the heart wall itself.

Compound models are optionally displayed as images (which includes the display as an image portion), for example images which combine structural anatomy with functional anatomy such as electrophysiological measurement results, and optionally computer-processed interpretations of electrophysiological measurements: for example, the location of the AV node, the location of the bundle of His, the location of a heart valve structure such as the valve annulus, and/or position along an imaginary axis over which the electrophysiological measurements themselves vary, for example as a function of waveform component amplitude and/or timing.

Potential advantages of applying a multimodal measurement approach to structural heart disease interventions is a reduction in how aggressive, risky, and/or expensive the overall procedure is. For example, in some embodiments, multimodal measurement provides information sufficient to guide the procedure without the use of methods that are performed with general anesthesia; for example, trans-esophageal ultrasound imaging. In turn, when general anesthesia is avoided, a requirement for artificial ventilation is potentially removed. Apart from adding to the complexity of the procedure, artificial ventilation has the effect of changing normal negative pressure breathing (sucking air in via movements of the chest and diaphragm) into positive pressure breathing (pushing air in artificially). Positive pressure, in turn, can have an effect on the shape of the heart, including shrinking valves that are normally more open and prone to regurgitation.

Consequentially, an annuloplasty procedure or valve clip implantation performed during positive pressure ventilation potentially under-corrects; or if positive pressure effects are taken into account, may paradoxically over-correct.

In some embodiments of the present disclosure, multimodal measurement removes a need to obtain a prior spatial map of the heart using CT or MRI imaging. Potentially, a need for planned open-heart surgery procedures and/or a risk of complications which lead to unplanned open-heart surgery procedures is reduced.

Reference is made herein to measurements of impedance, and to impedance signals, with the measurements being made using electrodes placed in intralumenal body locations. Particular reference is made herein to impedance measurements made in intracardial (heart) intralumenal locations such as the atria, ventricles, and the passageways leading thereto, therefrom, and/or therebetween.

The impedance measurements refer to electrical measurements configured to probe the electrical impedances of tissue structures near the measuring electrode or electrodes. Briefly, different compositions of matter (including, e.g., blood, connective tissue, muscle tissue, bone, lung tissue, and many other tissue structures found in the body) have different dielectric properties. These differences in dielectric properties in turn interact with electrical potentials so as to produce measurable differences in impedance.

Impedance is typically measured between pairs of catheter electrodes. For example, a catheter may be used which comprises a plurality of electrodes, and impedance can be measured between any (optionally every) pair of adjacent electrodes. The measurements may be made via use of a reference electrode; for example, the voltage between each of the two catheter electrodes in the pair may be measured against the reference electrode, and the difference between these two measurements can be taken as the voltage between the two catheter electrodes. The reference electrode may be, for example, a body surface electrode or patch attached to the patient's skin, for example, to the patient's leg.

In some embodiments, the impedance data may include impedance values for each electrode. For example, impedance at the position of an electrode may be calculated by averaging two impedance values, each measured between the electrode itself and another electrode adjacent to the electrode. The impedance measurement may be made via measurements of voltage. Measured voltage is generally indicative of impedance if the current, under which the voltage is measured, is constant over time, or varying with a stable pattern at some selected frequency. Thus, in some embodiments, the impedance measurements may be based on of impedance measurements directly, on voltage and current measurements, or on voltage measurements alone, any of which are optionally useful (suitably transformed and/or under suitable assumptions) as measures of impedance. In some embodiments, to obtain an impedance value for a pair of catheter electrodes, an alternating current of a given frequency is injected to one of the electrodes in the pair, and the voltage value measured at the same frequency between the two electrodes is divided by current measured on the electrode to which the current was injected. Optionally, the impedance value is obtained by other voltage and current measurements, for example, as described in International Patent Publication No. WO2019/215721.

Impedance can be analyzed and expressed in different ways known in the art, for example as complex numbers including a real part and an imaginary part; and or as impedance having a magnitude and a phase. Impedance is a frequency-dependent characteristic, with different compositions of matter, including different tissue types, producing different impedance measurements depending on a frequency used to probe impedance. Embodiments herein typically operate to measure impedance at frequencies between about 5 kHz and 200 kHz, but these frequencies are not limiting.

Impedance is closely interrelated with other electrical properties and parameters, including, for example, conductivity, complex conductivity, real or imaginary part of conductivity, magnitude or phase of conductivity, permittivity, complex permittivity, real or imaginary part of permittivity, and magnitude or phase of conductivity. To measure impedance within a region of the body, currents and/or voltages at controlled frequencies are introduced to the region. Impedance may be inferred, for example, by direct measurement of voltage potentials produced between electrodes upon the introduction of a controlled current, and/or by the direct measurement of currents produced upon the introduction of a controlled voltage.

For any given measurement, there may be many different contributing impedances that affect it. Impedances of particular structures can be isolated by use of differential techniques. In particular, two nearby electrodes in an intralumenal environment can be approximated as existing in the same impedance environment with respect to structures which are relatively far away from them (e.g., 10× further away from them as they are from each other). But they will be different from each other with respect to influences on impedance that are significantly nearer to one of them than the other. Thus, paired measurements can be used to particularly distinguish influences on impedance due to local features. The two electrodes can, for example, be used to measure independently, e.g., with respect to a common ground, or used directly in a sensing pair, e.g., with one electrode sensing and the other used as the ground.

In some embodiments, impedance readings made by different electrodes, or different electrode pairs, are normalized to reduce effect of differences between the electrodes or the pairs. For example, electrodes may differ from one another in structure (either because they are designed to have different shapes or sizes or because of manufacturing tolerances). When readings from two electrodes are used for calculating an impedance, the distance between the electrodes in the pair (e.g., along the probe) may differ between pairs. To normalize for these differences, the impedance readings are optionally divided by impedance readings of the same electrode (or electrode pair), received from the center of the lumen, away from any wall. In some embodiments, the readings from the center of the lumen may be identified based on positional information associated with the readings. In some embodiments, the center of the lumen may be identified using a tag provided by the operator. In some embodiments, the readings in the center of the lumen are identified based on the readings themselves, as the impedance in the center is generally smaller than near the walls. Thus, an impedance reading at, for example, one of the first 10 percentiles (e.g., first to tenth percentile) of the readings associated with an electrode or an electrode pair may be used for the normalization. These types of general normalization procedures may be assumed to be available for use in any of the embodiments herein, as appropriate. For some embodiments, more particular normalization methods (described therewith) may additionally or alternatively be used.

Reference made herein to “impedance signals” refers to time courses of impedance due to something changing in the electrical environment of a probing (measuring) electrode (other than the operating parameters of the sensing system of which it is a part). For example, an impedance signal may be induced when an electrode is moved closer to a tissue structure having a different (typically higher) impedance than the blood pool which otherwise surrounds it. Since the heart is in constant motion, there are generally many different simultaneous impedance signal sources affecting any given measurement. Extracting useful information from these multiple influences (e.g., information indicative of motion, including when the motion is itself indicative of the identity and/or characteristics of the signal source), in some embodiments, comprises control and/or analysis of the measuring position and/or measuring timing. For example, large (dominant) impedance signals are generally easier to attribute to a particular structure than small ones, other things being equal. Similarly, impedance signals measured under better known and/or better controlled conditions of positioning, contact, frequency, and/or timing (e.g., phase of a heartbeat or breathing cycle) are correspondingly more susceptible to analysis of what they might “mean” in terms of what structures are nearby, and/or what those structures are doing. For example, as already mentioned, impedance is typically measured at frequencies of several kilohertz. Accordingly, impedance signals superimpose upon alternating current and/or voltage induced by the probing circuitry. Since the probing circuit frequencies are known—and since, in any case, there is a wide difference between the higher frequencies of the probing circuitry and the lower frequencies of the impedance signals (whether or not they are repeating patterns), it is generally straightforward to make an analytical separation between the two. For impedance signals caused by different structures of the heart itself, an important general method to make a certain impedance signal stand out for use in analysis is to bring the probe electrode closer to the structure which is actually of interest. However, upon this general method, there may be added caveats—for example, constraints on where more precisely the measurement is taken from (e.g., at a small distance, and/or in stable contact). Phase of a heartbeat can also be an important factor in the characterization of an impedance signal, since many impedance signals associated with heart motion share similar frequency signatures. In a general sense, the heart's beating portions beat with the same fundamental frequency, but with different phases according to their placement and function. In some embodiments, measured electrophysiological signals are used to determine the phase of measured impedance signals.

Reference is also made herein to electrophysiological signals. These are distinguished by being caused by the endogenous electrical activity of the heart, as a part of its pumping function. Electrophysiological signals are referred to by several different terms herein—for example, electrocardiogram (ECG) and intracardial electrogram (IEGM). Use may also be made of the terms “body surface ECG”, “intracardial ECG”, and related terms. There may also be reference made to ECG “leads”. Each of these refer to the measurement of electrophysiological signals. In general, intracardial electrophysiological measurements involve the use of one or more electrodes positioned within the heart, while body surface ECGs refer particularly to electrophysiological measurements made of electrical currents that can be sensed using electrodes placed on the body's surface. Body surface ECG can produce some level of spatial sensitivity to what is happening inside the heart, generally through the use of multiple body surface electrodes placed in certain conventional fixed arrangements. Intracardial ECGs may likewise use fixed electrodes—e.g., implanted or otherwise positioned within the heart. However, intracardial ECGs (also referred to as IEGMs) are also measured using electrodes on probes, moved within the heart for carrying out a medical intervention. Measurements from moving electrodes (e.g., pairwise measurements) are used to sense the local electrophysiological environment of the electrodes, which potentially changes as the electrodes move. From this, it may be possible, for example, to identify that a certain electrode is near the AV node, or bundle of His; that it is in an atrium, a ventricle, or at the level of the valve in between (the “valve plane” or “AV plane”). Electrophysiological measurements may be referenced to each other in terms of their timing. For example, a body surface ECG may be used to measure the timing of standard heartbeat cycle events such as the P wave and QRS complex, and the relative offset between such an event and features measured by an intracardial electrode may be used to determine the position of the intracardial electrode inside the heart.

As many of the embodiments of the present disclosure describe, impedance signals may be interpreted with the use of electrophysiological signals. In some embodiments, this interpretation makes use of a third piece of information—where the electrode happened to be when a measurement was taken. While there is no particular limitation herein on how this positional information is acquired, a general class of methods of doing so involves inducing a plurality of different electrical fields within the heart that are distinguishable from each other, e.g., by frequency. Those electrical fields are then measured from one or more intracardial electrodes for which position is being determined. The measurement values provide information which can be used to locate those electrodes in respect to a frame of reference that the induced fields create, and/or in respect to each other. In a simple and non-limiting example, there may be three approximately orthogonal pairs of body surface electrodes inducing electrical fields across three approximately orthogonal axes. The resulting electrical fields can be used as coordinate axes to determine where the measuring electrodes are positioned. In practice, the determination of position may make use of additional information such as inter-electrode distances, e.g., to compensate for field non-linearities and uncertainties in what is known about the configuration of the field-inducing electrodes and the body portions therebetween.

In some embodiments of the present disclosure, tissue structures are identified using impedance signals, electrophysiology signals, and/or position information. The identification may be performed as part of a medical procedure being performed within a body lumen, such as a heart body lumen. In particular, the medical procedure may comprise an intervention to treat structural heart disease, for example annuloplasty, manipulation of the valve leaflets, closure of the left atrial appendage, valve replacement, or another procedure. Such procedures may also comprise procedure actions such as septal wall crossing, finding target, and/or assessment of the state of treatment targets before, during, and/or after treatment. The identification may be performed for the guidance and monitoring of such procedures. An identified structure or tissue structure may be characterized as part of or independently from identification. Characterization may be performed as part of the identification; for example, not only to identify a leaflet, but also to characterize some feature of the leaflet, such as its coaptation with adjoining leaflets. Characterization of the leaflets may be performed before, during and/or after treatment, and in particular may be performed before and after treatment, e.g., to assess treatment results.

An aspect of some embodiments of the present disclosure relates to the identification of motion signals carried within impedance measurement time series made using an electrode positioned within a body cavity, using a synchronizing indication. The motion signals comprise changes in impedance due to movement of a feature. A related aspect of some embodiments of the present disclosure relates to the identification of motion signals carried within impedance measurement time series made using an electrode positioned within a body cavity, using electrocardiogram features characteristics of one or more locations as markers to guide measurement and/or analysis of the motion signals.

Electrocardiogram (ECG) time series data are an example of an optional source of a synchronizing indication. Intracardial electrograms may have features that characterize the position from which the electrogram was recorded, providing a location marker. In some embodiments, events in intracardial electrograms are compared with events in ECG body surface electrodes to determine a temporal offset between them. The temporal offset may itself be characteristic of the position from which the intracardial electrogram was recorded.

In some embodiments, the motion signal which these synchronization indications are used to characterize is produced by movements of intralumenal structures (e.g., valve leaflets in a heart) nearer to and/or farther from a sensing electrode. The movements change the environment through which measured electrical signals propagate. In overview, the synchronizing indication is used to select periods during which the motion signal is expected to exist (if it is detectable) and/or have one or more particular features. This assists motion signal identification, for example in embodiments where the motion signal is not sufficiently predictable in waveform, where the expected feature is not confidently attributable to the motion signal without further information (e.g., a feature of a positive or negative slope in the time series data), and/or the motion signal is embedded in a time series comprising one or more potentially confounding signals (“noise”).

In some embodiments, the body cavity comprises one or more heart chambers, and the electrode is an intracardiac electrode. Optionally, more than one impedance time series is measured concurrently, using different frequencies and/or electrode combinations. Each impedance measurement time series may comprise, for example, impedance measurements made between an intracardiac electrode and a body surface electrode, and/or between a plurality of intracardiac electrodes. Frequencies at which impedance is measured optionally include frequencies between about 5-200 kHz.

Herein, reference to “impedance measurements” should be understood to include measurements of electrical signals which change as a function of impedance change, and/or are indicative of impedance. The electrical signal can be sensed, for example, via one or more electrical circuit properties such as voltage, current, resistance, and/or reactance. For example, impedance measurement is optionally performed by measuring voltage in a constant-current condition, and converted to impedance using electrical circuit analysis techniques (with body tissue and/or fluid as part of the circuit). Additionally or alternatively, voltage measurements are used directly as a proxy for impedance. Resistance and electrical current measurements are also optionally used additionally or alternatively to impedance measurements as such.

In some embodiments, a motion signal, once identified, is further processed for use in characterizing the position and/or the movement of a moving tissue structure; for example, to determine an electrode-to-structure distance and/or direction, to describe movements of the tissue structure itself, and/or to map surfaces of the tissue structure (that is, reveal geometry of the tissue structure). The identified motion signal is optionally represented, for example, as a waveform, amplitude, derivative of the waveform, and/or vector of amplitudes and/or derivatives of the waveform (i.e., amplitudes and/or derivatives taken from particular sampling periods of the impedance measurement time series). Although embodiments herein are described particularly with relation to moving tissue structures, it should be understood that they are optionally applied additionally or alternative to sensing of non-tissue structures such as implants with moving parts, for example, certain designs of artificial heart valve.

An intracardiac electrode measuring impedance is simultaneously subject to influences from a plurality of concurrently active signal sources. Some of these signal sources may be characterized as “motion signal” sources, which together contribute to make the time series of impedance measurements non-constant (i.e., varying in amplitude from moment to moment) due to movements of the heart. The frequencies involved in motion signal sources are generally low (most spectral power occurring at less than 100 Hz) compared, e.g., to the frequencies of several kHz or higher at which impedance is being evaluated.

The motions giving rise to the motion signals comprise, for example, motions of the heart walls and/or motions of structures (e.g., valves and/or their leaflets) within the heart chambers. There are potentially other movement contributions, for example due to motions of organs beyond the heart (e.g., lung movements), and/or due to changes in blood volume as a function of time. Other signal contributions may exist due to non-movement related reasons, for example, changes in electrode tissue contact.

The signal generated from each individual movement signal source potentially carries information about how the movement signal source is moving, where it is (e.g., over time), and/or its shape. However, interpretation of this information is confounded by simultaneous inputs from other sources. Moreover, the movement signal itself may be sufficiently variable in form (e.g., in its amplitude, phase, and/or frequency components) as to preclude ready identification of the signal from its own typical characteristics. This is particularly true for motion signals measured from a multiplicity of locations, such as may be done, for example, to gather data for generating spatial maps (reconstructions) of the motions and/or structures that give rise to the motion signals. Accordingly, there is a general problem of how to identify individual motion signal components from an impedance time series.

For a particular moving structure, the general problem may potentially also be particularized by bringing to bear special knowledge about how, when, and/or where the moving structure moves. This special knowledge may allow the impedance motion signal from the particular moving structure to be identified well enough to allow position and/or movement characterization of the particular moving structure. There may be preferable locations from which to make certain measurements (e.g., of valve leaflet movements), and electrophysiological signals may be a useful marker of such locations, such as the location of a valve annulus.

In some embodiments of the present disclosure, a particular moving structure of interest comprises valve leaflets, chordae, and/or papillary muscles of the heart. For these examples, one source of “special knowledge” is knowledge of the phase of overall heart contraction movements which is concurrent with and/or at a known time-offset from one or more characteristic movements of the moving structure of interest. For example, the mitral valve leaflets move to close against one another upon the onset of left ventricular contraction, and open during left atrium contraction. These contractions are in turn initiated by electrical signals that also generate characteristic electrocardiogram (ECG) features, such as the QRS complex and/or the P-wave. Accordingly, from knowing the time of occurrence of certain relevant ECG signal features, it is possible to infer a window of time during which certain motion signals may occur. In effect, the ECG signal is used, in some embodiments of the present disclosure, as a synchronization indication, helping to identify a particular structure's impedance-measured motion signal from confounding motion and other signals contributing to an impedance measurement time series. The use of information from the impedance-measured motion signal may provide more particular information about motion of a moving structure of interest—where it is moving, how much it is moving, and/or its shape, for example.

An ECG provides a particularly suitable source of a synchronization indication, in part because it provides a well-characterized sequence of features having well-understood time relationships to the movements of the heart overall. This optionally applies not only to normal ECG waveforms; for example, abnormal and/or irregular ECG waveforms may be associated with corresponding abnormal and/or irregular patterns of heart movements. The ECG information used may be body-surface recorded and/or intracardially recorded.

The synchronization indication is not limited to the heart ECG—it could comprise, for example, sensing of the time of a pressure or volume change (e.g., from a finger-attached pulse sensing device), and/or sensing of a heart noise (for example, the “lub-dub” noise of heart valves closing). Optionally, a synchronization indication is provided from ultrasound measurements; e.g., based on the timecourse of a sound Doppler signal related to valve and/or heart blood flow movements. For example, peak and/or minimum shift amplitudes of a blood flow Doppler signal may be synchronized with movements of a heart valve as it opens and/or closes.

It is noted that while the features of each heartbeat repeat regularly, there are still potentially significant beat-to-beat variations in timing (e.g., adjacent heartbeats are of slightly different lengths). Some particular synchronization indications are of particular value for particular movements, because, e.g., they are in a highly predictable cause-and-effect relationship (for example, valve closure causes heart sounds), and/or because they are constrained by physiology to coincide and/or succeed each other with a delay short enough that any physiological variability in relative timing can be disregarded as negligible.

To a first approximation, impedance motion signals measured from an electrode placed near to a particular moving structure increase in amplitude as a function of increased proximity to the moving structure. Distance being held equal, increased movement amplitude also corresponds to increased motion signal amplitude. In either case, the function may comprise, for example, an approximately inverse-power proportionality of motion signal strength to distance. Normalization, e.g., based on relative position within an X-Y coordinate plane parallel to the plane of a heart valve annulus, is optionally used to at least partially distinguish proximity effects on motion signal amplitude from movement amplitude as such. For example, leaflet locations near the periphery of a heart valve move less freely than leaflet locations more centrally positioned.

Without commitment to a particular theory of operation: in general, solid tissue structures are relatively electrically less conductive than blood, or even substantially non-conducting (at least as measured at certain frequencies). Thus, as they loom larger upon approaching an electrode, these tissue structures produce an increase in impedance, as they block off more and more of the potential paths for current to flow along. Over the course of a heartbeat cycle, a tissue structure such as a valve leaflet may successively approach and then retreat from a nearby measurement electrode (potentially a plurality of times), producing corresponding peaks and valleys in the time series impedance measurements. The impedances will tend to be larger, the closer the measuring electrode is to the moving structure. It may be noted that when two electrodes on a probe perform impedance measurements at the same time, the difference in impedance signal amplitude potentially provides direction information (e.g., the moving structure is more in the direction of the electrode sensing a larger motion signal). Moreover, the spacing distance of the electrodes is optionally used to help relate differences in sensed motion signal amplitude to the distance of the motion signal source.

As already mentioned, other parameters such as distance remaining equal, the amplitude of the impedance changes is potentially larger for a given increase in the range of motion. The amplitude may also be affected by non-linearities in the change in impedance as a function of electrode-structure distance—the impedance may change most quickly as a function of distance for movements which happen correspondingly closer to the measurement electrode.

Relating measured signals to the distance amplitudes of the motion they indicate may be difficult in a complex structure such as the heart, where many structures are moving at once, and where distance-related movement signals can be highly non-linear. However, proximity detection can still provide a valuable tool for mapping, since close-in structures are likely to produce larger signals as they move, potentially dominating over signals from more distant movements, so as to become less easily missed or mistaken. A potential opportunity arises in the case of the valve leaflets in particular, since one of their clinically important aspects is how well the leaflets coapt (seal against each other) when they have been pushed upward toward the valve annulus, e.g. (for the mitral and tricuspid valves), during ventricular contraction. Poor coaptation leads to regurgitation and weakened heart function. Locations approximately on the valve plane are well situated to allow impedance mapping here, since this is also near the position of leaflet coaptation.

In some embodiments of the present disclosure, electrophysiological measurements are used to establish measurement locations at or near the valve plane. This optionally defines a planar or plate-like (“thin block” shaped) region, which is not only in a well-defined anatomical relationship with the valve leaflets, but also quite close to them during certain parts of their movement cycle. In some embodiment, the planar region is established by making intracardiac electrophysiological measurements of intra-cardiac ECG features that vary characteristically along an imaginary axis extending transversely through the valve plane (e.g., the atrioventricular axis or AV axis). This characteristic variation is used to identify which region should be used as the reference area from which measurements of the valve leaflet motions are to be made. Once defined, position measurements made using a separate position-finding sensing modality can be used to guide movement of a sensing electrode into position, confirm positioning, and/or act as a filter to gate the use of just those impedance measurements which are positioned appropriately.

Because of the proximity of leaflets and valve plane, a strong impedance signal contribution from the valve leaflets is obtained at the valve plane. Moreover, as a reference, the valve plane has further advantages, including:

It is defined across a wide area, so that it can directly guide measurements across the whole surface of a valve.

It is so clearly defined as to promote reproducible identification within the same patient, and across different members of patient populations.

An aspect of some embodiments of the present disclosure relates to using the timing of events within a first electrical measurement time series to assist in generating a reconstruction of cardiac geometry using a motion signal recorded in a second electrical measurement time series.

In some embodiments, different (e.g., first and second) electrodes are used for each of the time series. In some embodiments, the first electrode measures ECG time series data. In some embodiments, the second electrode measures impedance time series data, and the motion signal comprises fluctuations in the impedance time series.

The present invention includes, in some embodiments thereof, methods and apparatuses for determining location of an intrabody catheter based on electrical measurements, for example, impedance measurements. More particularly, but not exclusively, embodiments of the present invention relate to determining which heart wall is being contacted with the intrabody catheter.

The methods may be used, for example, during a catheterization procedure, or after the procedure, for post-facto analysis. In some embodiments, catheters are used which are in the body for other purposes, and the method does not include inserting or manipulating the in-body catheter or electrodes. In some catheterization processes, the physician enters a heart chamber with a catheter and approaches a wall of the heart chamber, without certainty as to which wall it is, and/or needing to verify that the catheter is attached to a target wall in particular, and not any of the other walls. Walls may be distinguished, for example, according not only to where they are from a vantage within a current heart chamber, but also on the basis of what lies behind them.

For example, in many minimally invasive structural disease interventions, the catheter is introduced into the right atrium, and touches a wall of the right atrium. In some interventions, the procedure includes puncturing the wall between the right and left atria in order to intervene with structures in the left atrium. Accordingly, in some catheterization procedures it may be important for the physician to ensure the catheter touches the septal wall separating the right atrium from the left atrium (e.g., in preparation for crossing the wall), and not, for example, separating the right atrium from the aorta (which, if punctured in place of the septal wall, would create a serious and potentially deadly medical complication). In such cases, some presently disclosed embodiments may be useful to identify the heart wall touched by the catheter.

A method to identify a heart wall according to the presently disclosed technology includes accessing measured impedance data, and comparing them to reference impedance data. The heart wall is then identified based on the comparison.

In more detail, the impedance data includes impedance or voltage values collected using electrodes touching the heart wall to be identified. Preferably, the voltage or impedance is measured between pairs of catheter electrodes. For example, the catheter may be a catheter comprising one or more loops or coils bearing several electrodes (e.g., a Lasso™ catheter), and impedance can be measured between any (optionally every) pair of adjacent electrodes. The measurements may be via a reference electrode, for example, the voltage between each of the two catheter electrodes in the pair may be measured against the reference electrode, and the difference between these two measurements can be taken as the voltage between the two catheter electrodes. The reference electrode may be, for example, a body surface electrode or patch attached to the patient's skin, for example, to the patient's leg.

In some embodiments, the impedance data may include impedance values for each electrode, for example, in the above example of a looped and/or coiled catheter, each electrode may be associated with an average of two impedance values, each measured between the respective electrode and an electrode adjacent to the respective electrode. The impedance data may include measurements of voltage. Measured voltage is generally indicative of impedance if the current, under which the voltage is measured, is constant over time, or varying with a stable pattern at some selected frequency. Thus, in some embodiments, the impedance data may include impedance measurements directly, voltage and current measurements, or voltage measurements alone, any of which are optionally useful (suitably transformed and/or under suitable assumptions) as measurements of impedance. In some embodiments, to obtain an impedance value for a pair of catheter electrodes, an alternating current of a given frequency is injected to one of the electrodes in the pair, and the voltage value measured at the same frequency between the two electrodes is divided by current measured on the electrode to which the current was injected. Optionally, the impedance value is obtained by other voltage and current measurements, for example, as described in WO2019/215721.

In some embodiments, the catheter includes more than two electrodes, for example, 10 or 20 electrodes, or an intermediate number, and impedance data may be obtained from one of the electrode pairs, from each electrode pair, or from any subset of electrode pairs; preferably, pairs of adjacent electrodes. The impedance data may include the voltage and/or impedance measurements, associated with indication to the electrode pair, for which the voltage and/or impedance value was measured.

The impedance data is collected during at least one heartbeat, and preferably includes indications to the heart beat stage at which each impedance value was measured. In this respect, a heartbeat stage is the time along a heartbeat at which the value was measured. For example, the time along the heartbeat may be characterized by synchronization with body surface ECG signals received at the same time as the impedance measurements. Exemplary time stages include the time of the QRS complex peak, the T wave, etc. In some embodiments, the time stage may be characterized by the time that lapsed from the beginning of the heartbeat to the measurement of the impedance data, in units of heartbeat length. The beginning and ending of a heartbeat (between which a heartbeat length is defined) may be determined based on ECG signals. Examples of such heartbeat stages include, for example 1/100, 2/100, 1/10, half, 90% heartbeat, etc.

The comparison of the impedance data may be to reference data, which include or represent impedance measurements made using a similar catheter touching an identified heart wall. The heart wall identification used for generating the reference data may be, for example, imaging based. Many such measurements (e.g., measurements taken during more than 10 heartbeats when touching each heart wall by 10 electrodes) may be included in the reference data.

The comparison of the impedance data to the reference data may be by a supervised learning algorithm, for example, SVM (support vector machine). For example, during a heartbeat, impedance values may be obtained at N heartbeat stages (e.g., 50 times in a heartbeat) for each of M electrodes (e.g., 10 electrodes) that touched the heart wall during the entire heartbeat. This set of M×N (e.g., 500) impedance values may be viewed as a point in a space with M×N dimensions. The reference data may include many such points, each associated with one of the heart walls. The measured impedance data can be viewed as another point in the same M×N dimensional space. The identification of the wall may be determined, for example, by calculating the distance (in the high-dimensional space) between the measured data and the reference data associated with each of the walls, and identify the wall as the one, to which the reference data is closest. In some embodiments, the high dimensional space is divided into two (or more) parts, each associated with a wall (or one associated with a target wall, and the other with any non-target wall, etc.) and the heart wall is identified by the space-part at which the measured data fall.

In some embodiments, instead of comparing points in such high a dimensional space, the dimensionality of the problem is reduced by representing both the reference data and the measured data by a set of characteristic features. Examples of characteristic features include a time average of the impedance associated with an electrode (there may be M such features), variance of the impedance associated with an electrode (there may be M such features too), the impedance values measured by an electrode at k (e.g., 7) predetermined stages in the heartbeat (there may be k×M such features), the time derivative of the impedance value measured by an electrode at p (e.g., 6) predetermined stages in the heartbeat (there may be p×M such features), etc.

In some embodiments, for each wall and electrode, a characteristic function of impedance vs. time is determined, and for each data segment that includes impedance measured during a single heartbeat by a single electrode, a local function of impedance vs. time is determined. The distance between the two functions may be determined, for example, as the average distance between the impedance measured at a selection of heartbeat stages and the reference impedance associated with the same heartbeat stages. The distance between the local function and the characteristic function may be a feature used in comparing the measured data to the reference data. The number of such features may be one for each electrode and wall, so if the number of walls is w (e.g., 4), the number of such features is w×M.

In some embodiments, to identify a heart wall, the time development of measured impedance data is compared to time development of the reference data. The comparison may include comparing characteristic features related to the time development of measured data to the corresponding characteristic features of the reference data, e.g., using a supervised learning algorithm. Examples of features related to the time development of measured or reference data include the impedance values measured by an electrode at predetermined stages in the heartbeat, the time derivative of the impedance value measured at predetermined stages in the heartbeat, difference between measured and reference impedance vs. time functions.

In some embodiments, features not related to the time development of the impedance, such as impedance average and/or variance over a heartbeat, may also be used for the identification of the heart wall.

It is noted that in some catheterization procedures, or during different times during a single catheterization procedure, contact between the electrodes and the heart wall may be partial. For example, some of the electrodes may touch the wall, while others don't touch it, touch it only for a fraction of the heartbeat cycle, touch it weekly, or touch it unstably. In such cases, in order to use only data collected by electrodes that actually touch the wall (or more particularly, those that touch it so as to record usefully stable data), the electrodes are first classified into touching and not touching electrodes, and data is collected only from electrode pairs that both touched the wall. An estimation if an electrode touches a heart wall or not may be obtained, for example, by inspection of the variance of the measured impedance. An electrode that does not touch the wall measures mainly the impedance of the blood around it, and this impedance does not change much during a heartbeat. Therefore, electrodes that measure during a heartbeat impedance that varies by less than a threshold may be considered as not touching, and taken out of consideration for identifying the heart wall. Conversely, very rapid changes in impedance (step changes) may indicate intermittent contact, even though the variance is high. Alternative ways of estimating if electrodes touch a wall or not may include contact quality assessment, as described, for example, in International Patent Publication No. WO2016/181315, U.S. Pat. No. 5,598,848, or U.S. Patent Publication No. 2010/0274239.

After the touching electrodes are identified, only data collected from them is used for comparison with the reference data to identify the heart wall. Optionally, a similar procedure is taken also when the reference data is prepared.

Another aspect of embodiments of the disclosed technology includes an apparatus configured to carry out a method as described herein for identifying a heart wall. The apparatus has a processor with access to one or more digital memories storing all the data required for identifying the heart wall. The processor is also configured, e.g., by appropriate programming, to compare the measured data with the reference data and identify the wall based on the comparison. The processor may also be configured to distinguish between touching and not-touching electrodes, and use for the identification of the heart wall only data collected by touching electrodes.

The apparatus may also have inputs for receiving impedance readings in real time. For example, it may include input connectible to the catheter, for receiving voltage, current, and/or impedance readings, and input for receiving readings from body surface ECG. In such embodiments, the processor may be further configured to associate (e.g., by time at which different data items were collected) between readings received via the two inputs, store them, and access the stored data for use as input for the comparison.

An aspect of some embodiments of the present invention relates to a method of indicating locations of a heart valve leaflet in an image of the heart or a part thereof, which includes the AV plane. The heart valve, in some embodiments, is an atrioventricular valve (i.e., mitral valve or tricuspid valve), situated between an atrium and a ventricle. The AV plane is an anatomical plane separating the atrium and the ventricle. The leaflets change their position continuously, and the method allows at least to indicate relative changes in their position when the valve is open and when the valve is closed. In some embodiments, leaflet locations when the valve is opening or closing (i.e., between closed and open states) may also be indicated. In some embodiments, the indication changes as the valve changes state from closed to open during each heartbeat. In some embodiments, this may be in real time. It is noted that the method indicates locations identified to be locations of the leaflets, regardless of the accuracy of such identification.

In some embodiments, the method begins with identifying a plane in the image as the AV plane. Such identification may be carried out based on IEGM signals measured in the body part by one or more electrodes of an intra-body probe. Identification of different locations in a heart wall as belonging to an atrium or ventricle based on IEGM (intracardial electrogram) signals measured by an electrode touching the heart-wall location and their synchronization with ECG (electrocardiogram) signals is generally known in the art. Such known methods may be utilized to identify various points as belonging to a ventricle or to an atrium. Once such identification is achieved, a plane separating locations identified as belonging to a ventricle from locations identified as belonging to an atrium. In some embodiments, this separating plane is identified as the AV plane.

Finding a plane separating between atrium points and ventricle points may be accomplished using machine learning models, such as support vector machines (SVM), and/or other optimization methods, for example, stochastic gradient decent.

It is noted that the heart walls move during a heartbeat, and the AV plane moves with them. In some embodiments, the image is a beating image, that changes over time to show the shape of the heart (or the imaged part thereof) as it changes during a heartbeat. For example, the image may be a 4-D image, i.e., a 3-D image that changes over time. In some such embodiments, the AV plane is identified separately for different heartbeat phases, and shown to move together with the heart walls in the beating image.

In some embodiments, after the AV plane is identified, each of a plurality of image points residing in the vicinity of the AV plane is identified as a leaflet point or non-leaflet point. In some embodiments, the definition of the “vicinity” of the plane may be defined by a user, using a user interface. Alternatively or additionally, the vicinity may be pre-programmed, or may be programmed to be automatically determined on the fly, for example, considering the noise level of the measurements. Typically, a point may be considered in the vicinity of the plane if the point's distance from the plane is between 1 mm and 5 mm. It may be preferable to use a larger distance if the measurements are noisier.

The classification of the points on the AV plane to leaflet points and non-leaflet point may be based on impedance values, each being associated with a respective image point. For example, the image may include a point cloud, in which each points is based on measurements taken by electrodes of an intra-body probe. In some such embodiments, electrodes of the same probe may also measure impedance. For example, one or more of the probe electrodes may be used for exciting in the vicinity of the probe an electrical field, and one or more of the probe electrodes (optionally, different from the ones used for the excitation) may be used to measure that electrical field. Such measurements may be used to evaluate an impedance value, e.g., by dividing a voltage measured between two measuring electrodes by the electrical current measured to run between them. When the electrodes are at a certain location, the measured impedance may be associated with that location, e.g., with the location occupied by each one of the measuring electrodes during the measurement. Optionally or additionally, the impedance values may be associated with a location on the image, corresponding to the location of the electrode in the heart, when the measurement was taken. Such impedance values may be used for identifying the image points as leaflet points or non-leaflet points.

The distinction between leaflet points and non-leaflet points may be accomplished, for example, by setting a threshold, and identifying points associated with impedance values higher than the threshold as leaflet points, and points associated with impedance values lower than the threshold as non-leaflet points. In some embodiments, setting the threshold may be by an operator, for example, during execution of the method, and observing the quality of the image obtained at different threshold levels. In some embodiments, the threshold may be pre-defined, for example, as one standard deviation above the average of impedance values associated with points in the vicinity of the VA.

Once different points are identified as leaflet points, and others as non-leaflet points, the image may be displayed with locations corresponding to points identified as leaflet points being displayed differently than locations corresponding to points identified as non-leaflet points. For example, the leaflet points may be displayed at different color and/or transparency than the non-leaflet points. In some embodiments, the image may be a shell generated based on a point cloud, and the locations displayed differently are not points, but shell-portions associated with the corresponding points.

In some embodiments, the method may be used after data has been collected for several heartbeats. Each heartbeat may be divided to phases, e.g., to a predetermined number of phases. For example, the period between two R peaks in an ECG may be divided to a predetermined number of phases (e.g., between 20 and 30 phases). Alternatively, the heartbeat may be divided to phases based on further features of the ECG signal, for example, the various phases may correspond to the QRS complex, the P-wave, the T-wave, etc. In some embodiments, one or more of the ECG features may be divided to several phases (e.g., the P-wave to 5 phases, the T-wave to 10 phases, and the QRS may be treated as a single phase). When data collected a plurality of heartbeats is available, in some embodiments, data measured in different heartbeats during the same heartbeat phase (e.g., during the 50 ms following the R peak) is accumulated, and used to generate a single image. For example, the points identified as leaflet points during a single heartbeat phase, and in a plurality of heartbeats, may be displayed simultaneously, for example, for a period corresponding to the period of the heartbeat phase. In some embodiments, such displays are repeated, so that consecutive displays correspond to consecutive heartbeat phases, to generate a cine of the leaflets opening and closing during a heartbeat.

An aspect of some embodiments of the present invention relates to an apparatus configured to carry out a method as described above. In some embodiments, such an apparatus includes a memory, a processor, and a display. It is noted that the apparatus may include more than one of each of these parts, for example, a plurality of memories, processors, and/or displays. The invention is not limited to the number of memory devices used, for example. An apparatus including a memory storing information may be any device wherein the information is saved on one or more memories. Similarly, an apparatus including a processor that executes instructions may include a plurality of processors, that together execute the instructions.

In some embodiments, the memory stores the image of the body part, on which the leaflet and non-leaflet points will be marked or displayed. In some embodiments, the image may include a point cloud. Each point in the cloud may be a point in which an electrode of the electrode probe visited. In some embodiments, the image may include a mesh generated based on such a point cloud. In some embodiments, the image may be an MRI image, a PCT image, or any other image of a part of the heart that includes the AV plane. In some such embodiments, the processor may be configured to register between points or locations in the image with corresponding points or locations in the body, so as to allow identifying on the image the points at which the IEGM and/or impedance have been measured.

In some embodiments, the memory also stores IEGM data. The IEGM data may include a plurality of IEGM signals measured in the body part by one or more electrodes of the intra-body probe. The IEGM data may be synchronized with ECG data, obtained from body surface ECG measuring system, which may or may not be part of the apparatus. In some embodiments, the IEGM signals are synchronized with ECG data by recording the times at which each signal is being recorded. To be synchronized, the clocks used for recording the times of the two signals (in the present case, IEGM and ECG) are the same, or record the same times at the same instances. This allows associating each signal with a phase of a heartbeat, in order to identify the point at which the signal was measured as an atrium point or ventricle point. In some embodiments, the IEGM data includes the IEGM signals and association of each such signal with a respective location in the image. The respective location is a location in the image that correspond to the location in the body at which the electrode resided when it measured the IEGM signal.

In some embodiments, the memory also stores impedance data. The impedance data may include, for a plurality of image points, a respective impedance value, and an association between the impedance value and the respective image point. The image point associated with the impedance value is an image point that corresponds to a location in which the impedance value was measured. In some embodiments, the impedance is measured using a pair of probe-electrodes, and each electrode of the pair is associated with the impedance value measured using the pair.

The processor also stores instructions, that when executed by the processor cause the processor to identify the AV plane, as well as leaflet points and non-leaflet points in the vicinity of the AV plane. In some embodiments, a plane in the image is identified by the processor as the AV plane based on the IEGM data, and points in the vicinity of the identified plane may be identified as leaflet or non-leaflet points based on the impedance data.

The processor may also control the display to display the image with locations corresponding to points identified as leaflet points being displayed differently than locations corresponding to points identified as non-leaflet points.

In some embodiments, each IEGM signal in the IEGM data is further associated with a respective heartbeat phase. The heartbeat phases in the IEGM data may be different from the heartbeat phases in the impedance data. For example, the heartbeat phases in the IEGM data may be designed to allow identification of locations as belonging to atrium or ventricle. Thus, in some embodiments, there are three phases in each heartbeat: P-wave, QRS, and other. A point in the body, at which an electrode detected an IEGM spike during the P-wave only may be identified as an atrium point, while a point in the body at which an electrode detected an IEGM spike during the QRS only may be identified as a ventricle point. A point in the body at which an electrode detected IEGM spikes in both the P-wave and the QRS may be identified as lying on the AV plane. However, as such points may be seldom, their role in identifying the AV plane is not central, although may be taken into consideration for such identification. IEGM spikes are not expected between the QRS and the P-wave of the following heartbeat. Therefore, dividing a heartbeat to three phases (P-wave, QRS, and other) may be useful for identifying points as atrium points or ventricle points. However, for generating a cine as described above, more heartbeat phases may be advantageous.

Thus, in some embodiments, the instructions stored on the memory cause the processor to identify, based on the IEGM data, ventricle locations in the image associated with IEGM signals, the heartbeat phase associated therewith being indicative to touching a wall of a ventricle, and atrium locations in the image associated with IEGM signals, the heartbeat phase associated therewith being indicative to touching a wall of an atrium.

In some embodiments, the processor is configured by the instructions to identify a plane separating ventricle points from atrium points as the AV plane. Such a separating plane may be identified, for example, using SVM model and/or stochastic gradient decent method.

In some embodiments, the apparatus may be configured to generate a cine of the leaflets as they open and close the valve during a heartbeat. In some such embodiments, the impedance data further comprises a respective heartbeat phase associated with each of the plurality of image points, and the instructions cause the processor to access the impedance data and cause the display to simultaneously display locations corresponding to points identified as leaflet points in different heartbeats during a common heartbeat phase. For example, the display may be of a “frame” showing as leaflet points, points identified as leaflet points during 10 different heartbeats, at the first 20 ms after the QRS in each heartbeat.

In some embodiments, the instructions cause the processor to repeat causing the display to display such frames, so that frames displayed consecutively correspond to consecutive heartbeat phases.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Annuloplasty Device Implantation

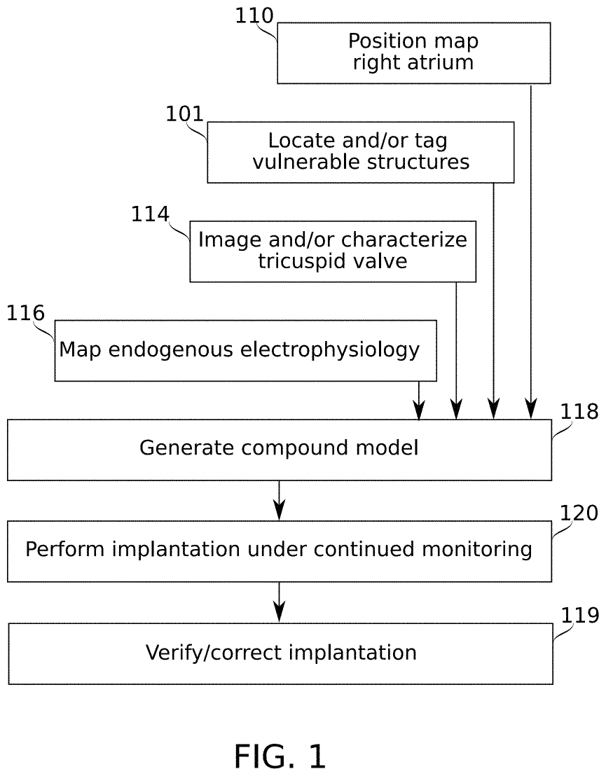

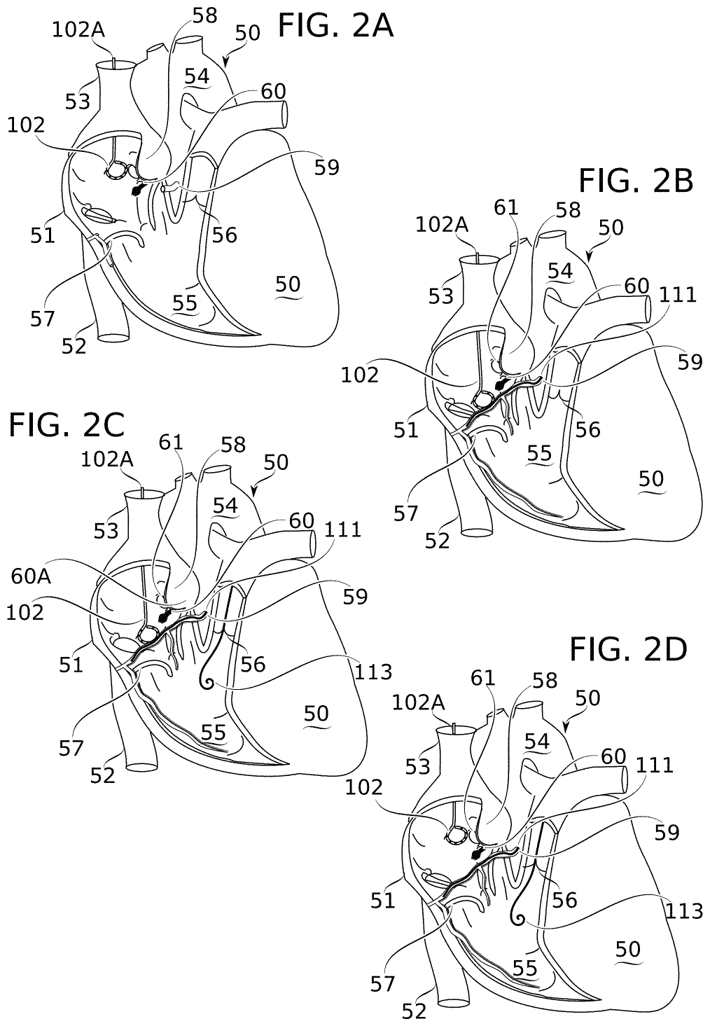

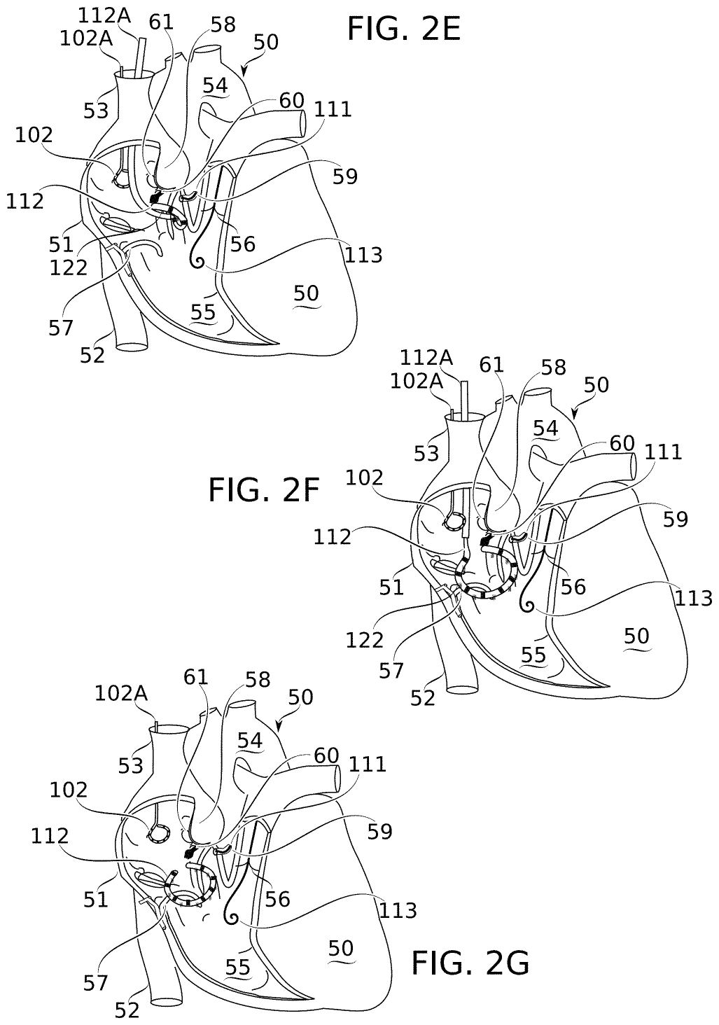

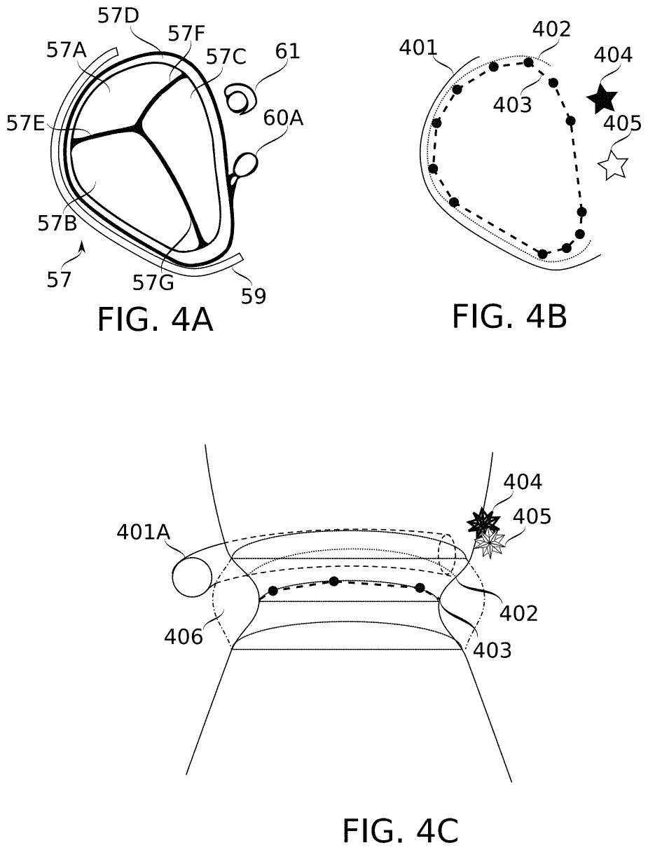

Reference is now made to FIG. 1, which is a schematic flowchart of a method of guiding and monitoring implantation of a tricuspid heart valve annuloplasty device 112 using a multimodal measurement approach, according to some embodiments of the present disclosure. Reference is also made to FIG. 6, which schematically represents an annuloplasty device 112, according to some embodiments of the present disclosure. Additional reference is made to FIG. 4A, which schematically represents an overhead view (looking from a right atrium 51 toward a right ventricle 55) of a tricuspid valve 57, according to some embodiments of the present disclosure. Further reference is made to FIGS. 2A-2G, which schematically illustrate selected phases in the implantation of annuloplasty device 112, together with examples of auxiliary tools used to guide and/or monitor the implantation, according to some embodiments of the present disclosure.

An implantable device used in tricuspid annuloplasty, in some embodiments, comprises annuloplasty device 112 (e.g., as shown in FIG. 6), which is attached to tissue extending around a circumference of a tricuspid valve 57, and then actuated (e.g., altered in shape by shrinking) to modify function of the tricuspid valve 57. The modification is aimed at reducing regurgitation through the valve. In some embodiments, the attachment is performed by inserting fasteners 122 (e.g., screws, coils, or another anchoring device) into tissue surrounding the tricuspid valve 57, for example from within a sleeve 121 of the device. Actuation of the annuloplasty device 112, in some embodiments, comprises cinching of cord 125. This causes the circumference of annuloplasty device 112 to reduce. In accord with this, the circumference of tricuspid valve 57 itself is reduced (optionally after a period of remodeling). This potentially reduces regurgitation through tricuspid valve 57 by bringing the leaflets 57A-57C into closer apposition (coaptation).

The implantation is performed, in some embodiments, using minimally invasive (e.g., over-catheter) techniques of delivery, positioning, deployment, and/or attachment. Approaches to the heart for minimally invasive procedures include, for example: vascular approaches via the inferior or superior vena cava; through arteries (e.g., from the carotid artery or by small chest incision); or in some embodiments through the apex of the left ventricle.

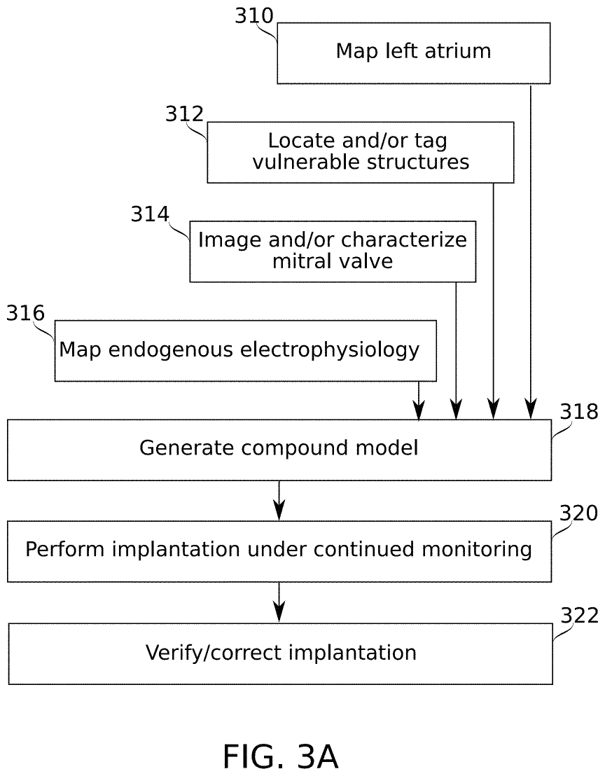

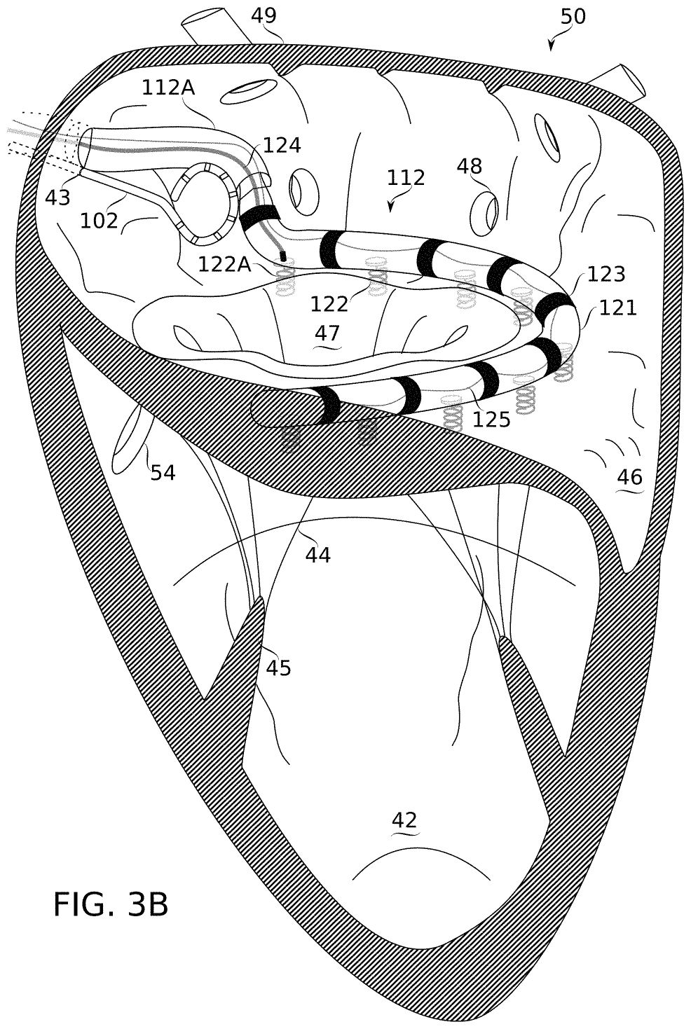

A range of problems (described further in the descriptions following) are associated with implantation of annuloplasty devices (e.g., the tricuspid valve annuloplasty method of FIG. 1, and/or the mitral valve method annuloplasty method of FIG. 3A). These problems potentially interfere with safety, reliability, and/or effectiveness of the device and/or the procedure which implants it. In some embodiments, problems are potentially mitigated by the measurement and use of data which indicate aspects of the anatomical and functional environment of the device at the site of implantation, and/or aspects of the device itself.

With respect to the anatomical/functional environment, data may indicate, for example, overall heart lumen shape, overall heart function, heart structural anatomy, and/or heart functional anatomy. Although these categories are not dichotomous (the same data potentially belongs to more than one of these categories), the categories mentioned represent differences in emphasis.

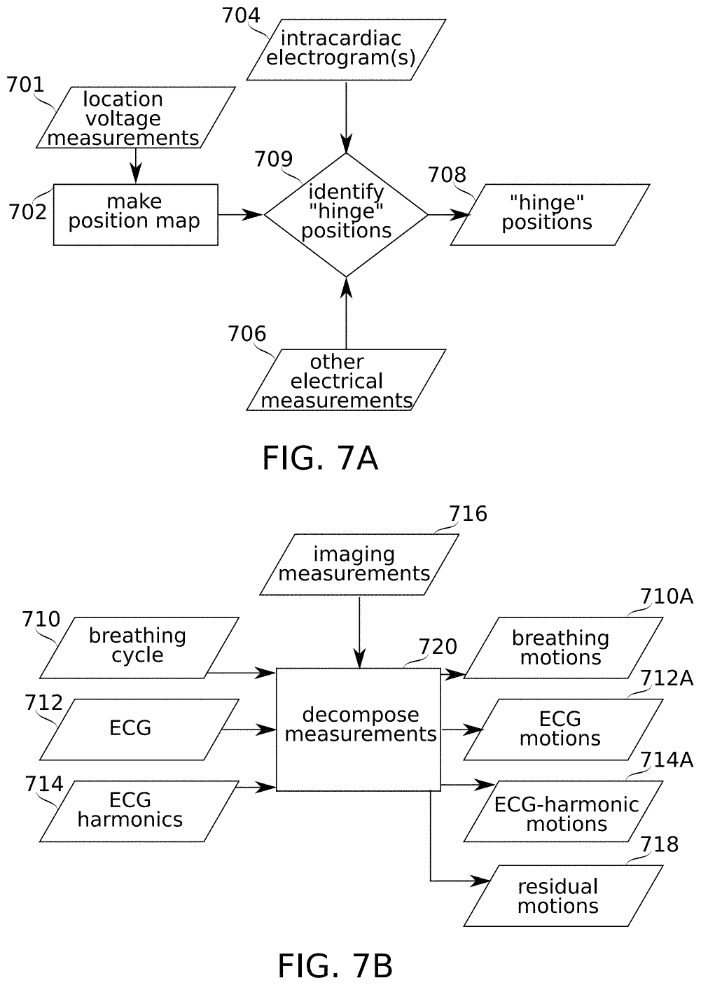

In particular, data indicating heart structural anatomy optionally encompass one or both of (for example):
Locations of regions and/or boundaries of heart tissue, defined based on distinctions in mechanical and/or cellular-level properties. These optionally include, for example: the boundary between fibrous tissue of the valve annulus 57D and surrounding cardiac muscle, the boundary between valve leaflets and the valve annulus 57D, and/or the course of transmission pathways such as the bundle of His 60. FIG. 7A provides an example.
Specifics of cardiac shape including the detailed shape and/or location of lumenal and/or perilumenal structures. These optionally include, for example: papillary muscles, chordae, valve leaflets, valve annuli, cardiac structures specialized for impulse transmission (e.g., sinoatrial (SA) node, atrioventricular (AV) node 60A (for example, as described in relation to FIGS. 2C and/or 4A), and/or bundle of His 60), blood vessels supplying and draining the cardiac tissue (coronary arteries, coronary veins), and/or other major blood vessels.

While some of these may be deduced in part from overall heart lumen shape, “overall heart lumen shape” as such refers herein to the (optionally time-varying) shape of the lumenal wall boundary as such (e.g., the bounds of movement of an object within the lumen), without specific reference to tissue properties.

Data indicating heart functional anatomy optionally encompass one or more of (for example):
Passive and/or active details of how structures move; for example, movements of valve leaflets and/or contractions of cardiac muscle.
Electrical activity of cardiac tissue, optionally including variations over time and space.
Measurements that provide a metric for a function attributable to a specific heart structure: for example, a measurement of the backward flow of blood through the defective tricuspid valve (known as tricuspid valve regurgitation) may characterize the functional anatomy of the tricuspid valve 57, not necessarily with structural detail. The tricuspid valve regurgitation may be measured, for example, by sensing backflow of an injected tracer fluid such as saline or dye.
Metrics of anatomy and/or movement associated with specific structures, and carrying special meaning for the operation of the heart. For example, an open area of the tricuspid valve 57 when it is maximally closed is structural, but also a metric of regurgitation. In another example: a percentage of shortening of the papillary muscles during the cardiac cycle (summarizing their dynamic motion) has potential implications for valve function such as risk of prolapse.

In contrast to such structurally-associated data, data characterizing “overall heart function” includes, for example: body surface ECG recordings, heart rate, and/or overall pumping volume of the heart.

Herein, a measurement modality processes measurement data with some processing device and/or method, thereby producing processed measurements particular to the measurement modality. Herein, the data may include, for example, indications of structural anatomy, functional anatomy and/or overall heart function. One or more analysis procedures are applied to extract specific information from the data; for example, measurements of electrophysiological function may be processed to identify positions of certain heart structures. Measurement modalities are not necessarily segregated from each other in both data and processing. For example, in some embodiments, the same tool (e.g., electrode-carrying probe), and optionally even the same stream of raw measurements (e.g., a stream of voltage measurements from an electrode of the electrode probe) is used as the basis of a plurality of measurement modalities. In such embodiments, the measurement modalities are distinguished from each other, for example, by different algorithmic processing, by auxiliary information used, and/or by how outputs are integrated to the multimodal model and/or presented for display. It should be understood, however, that measurement modalities are optionally grouped together according to their commonalities; for example, the measurement device and/or source of signal energy being measured (for example: electrodes/electrical fields, magnets/magnetic fields, ultrasound transducer/ultrasound generator, X-ray sensor/X-ray source). Blocks 110, 101, 114, and 116 of FIG. 1 (and corresponding blocks 310, 312, 314, and 316 of FIG. 3A) relate to certain specific measurement modalities. Integration of measurement modalities (to the compound model) is further discussed herein, particularly with reference to block 118 of FIG. 1 and/or block 318 of FIG. 3A.

With respect to the device itself: data may indicate, for example: device position, device orientation, device deployment status, and/or device attachment status. Data may also indicate, directly or indirectly, how the device interacts with the anatomical environment, e.g., to affect (actually, as estimated, and/or as predicted) cardiac function. The interactions measured are potentially intended and/or unintended; therapeutic and/or adverse. Herein, measurements related to device status are discussed with reference, for example, to blocks 120, 119 of FIG. 1, and/or blocks 320, 322 of FIG. 3A.

In some embodiments of the present disclosure, implantation and/or validation of implantation is guided and/or monitored using within-body devices for imaging and/or sensing. In some embodiments, this is without the use of ionizing radiation (e.g., without the use of X-ray and/or radionuclide-based imaging).

A particular potential advantage provided by some embodiments of the present disclosure is conferred by use of electrode measurements to provide a plurality, and optionally substantially all, of the data used in the different measurement modalities. For example, an electrode used in mapping positions within a cardiac lumen is optionally used also for sensing:
differences in dielectric properties characteristic of different tissue structures, and/or contact therewith,
dielectric signals characteristic of an injected tracer (such as saline),
intrinsic cardiac electrical activity, and/or
electrical signals transmitted by a marker device such as a wire inserted to the coronary artery 59.

Potential advantages of using electrical measurements, compared to other intrabody probe types, include reduced numbers of probes, and/or increased simplicity of probes. Optionally, the implantation catheter 112A (FIGS. 2E-2F; FIG. 3B) and/or the implanted annuloplasty device itself and/or its attachment hardware includes at least some of the measurement electrodes, for example, electrodes positioned along a body of the catheter, potentially reducing a number of catheters to be inserted during the procedure. There is also a potential advantage for data integration, for example, insofar as data for two different measurement modalities can be directly identified as characterizing the same location, for example, if they were recorded by the same probe at the same time. There are also potential advantages in terms of implementation, by reducing complexity in coordinating between disparate measurement devices.

Some descriptions herein relate to a respective operation (action/s within a procedure) performed in a certain manner, to achieve some particular intermediate result of an overall implantation procedure. It is to be understood that such operations are optionally performed within any suitable overall procedure, and performed, moreover, in any suitable combination with other operations of the procedure, and in any suitable order, as may be selected to achieve the implantation. For example, some operations are described as one option among a plurality of options for accomplishing the same intermediate result; any procedure which includes accomplishing that intermediate result optionally uses any of the plurality of options. In some embodiments, the intermediate result itself is an optional part of the overall procedure—for example, an operation to verify position and/or attachment may be performed optionally. Moreover, in some embodiments of the present disclosure, all or portions of operations optionally occur sequentially (and the particular order of the sequence itself is optionally defined) and/or in parallel (e.g., simultaneously) with each other. This applies, in particular but not only, to operations described in relation to blocks 110, 101, 114, and/or 116 of FIG. 1 (and/or blocks 310, 312, 314 and/or 316 of FIG. 3A).

FIG. 1 describes general classes of operations which are optionally performed in some embodiments of the present disclosure, relating them generally to measuring, mapping, and positioning devices. FIGS. 2A-2D (and their associated descriptions) relate more specifically to operations which measure and help define the anatomical environment (and may comprise instances of operations of FIG. 1). FIGS. 2E-2G (and their associated descriptions) relate more specifically to operations during the implantation of an annuloplasty device 112, and/or to validation of the implantation (which may comprise instances of operations of FIG. 1).

### Position Mapping

At block 110, in some embodiments, the right atrium 51 is measured, and the measurements converted to a position map indicating the shape of at least portions of the internal lumen of right atrium 51. Optionally, position measurements of the tricuspid valve 57 and/or right ventricle 55 are also made within the actions of block 110 (although measurements of tricuspid valve 57 are related to more specifically in relation to block 114).

The position map, in some embodiments, defines (e.g., is used to estimate by computer-implemented processing) a shape of an interior surface of one or more cardiac lumens of a heart. In some embodiments, the position map defines the shape of a volume limited by interior surfaces of the one or more cardiac lumens. The position map is optionally dynamic (e.g., defined as a function of heartbeat and/or respiratory phase) and/or processed (e.g., from dynamic data) to produce a static position map (for example, by use of gating and/or a process of frequency component decomposition; e.g., as described in relation to FIG. 7B, herein).

Position mapping comprises, in some embodiments, movement of a multi-electrode catheter probe 102 (FIG. 2A) within the lumen of, for example, the right atrium 51, while making electrical measurements (e.g., of voltage, current, and/or impedance) of a plurality of exogenously generated (that is, artificially generated) electrical fields which “tag” the volume within which the electrode catheter probe moves, and/or establish an electric field-defined coordinate system within the heart. The electrical fields are optionally generated at different frequencies, and/or multiplexed in time so that they can be distinguished from each other. Each electrical field is generated using an electrode set comprising a plurality of electrodes; the sets of electrodes used for generating the different electrical fields are at different body surface and/or internally situated positions, so that the resulting electrical fields have gradients crossing within the heart in different directions. This causes a different set of measurements to be obtained at each measurement location, such that the set of measurements is characteristic of the location.

The shape of the lumen, in some embodiments, is modeled by a process of reconstructing positions from the measurements. Optionally, a plurality of electrodes of the multi-electrode catheter 102 are operated to make simultaneous measurements. Optionally, reconstructing the positions comprises making use of known inter-electrode distances to constrain a solution which associates each set of measurements with a particular position in space, for example as described in International Patent Publication Nos. WO 2019/035023 A1, entitled FIELD GRADIENT-BASED REMOTE IMAGING; WO 2018/130974 A1, entitled SYSTEMS AND METHODS FOR RECONSTRUCTION OF INTRA-BODY ELECTRICAL READINGS TO ANATOMICAL STRUCTURE; and/or WO 2019/034944 A1, entitled RECONSTRUCTION OF AN ANATOMICAL STRUCTURE FROM INTRABODY MEASUREMENTS—the contents of each of which are included by reference herein, in their entirety.

In some embodiments, another method is used to position map the right atrium 51 and optionally associated structures such as the left ventricle 55. For example, mapping is performed using a different electrical field imaging method, ultrasound, X-ray imaging (fluoroscopy), CT imaging, MRI imaging, and/or magnetic field imaging. X-ray images, for example, may be used to establish a background within which positions and movements of other procedure elements (e.g., catheters, probes, and/or the annuloplasty device 112 itself) are visualized during the procedure. In the case of ultrasound imaging, a probe at the illustrated position of multi-electrode probe 102 may optionally or additionally comprise an ultrasound imaging probe.

In the case of magnetic imaging, a probe at the illustrated position of multi-electrode probe 102 may optionally or additionally comprise a magnetic imaging sensor (e.g., coil). In the case of electrical field imaging methods, a probe at the illustrated position of multi-electrode probe 102 may optionally be a loop (lasso) probe such as is illustrated, or another multi-, dual- or single-electrode catheter probe, for example one with electrodes arranged along a corkscrew (which potentially provides an increased sensitivity to depth along an imaginary axis parallel to the longitudinal axis of the corkscrew), or a catheter probe configured closer to a wheel-and-axle configuration, with the loop of the “wheel” approximately centered on the main body of the probe.

In some embodiments, the position-measuring probe is positioned outside the heart (e.g., inside the esophagus in the case of transesophageal echocardiography (TEE)), and optionally outside the body (e.g., on the chest in the case of transthoracic echocardiography (TTE)). It is noted that successful imaging with ultrasound imaging techniques is difficult to achieve in certain cases, for example due to a patient-specific configuration of anatomy that interferes with visualization. In some embodiments, the position measuring probe is positioned in the coronary artery.

The position mapping of block 110, in some embodiments, is expanded to include all or portions of other heart chambers, blood vessel portions, and/or other heart structures for example, right ventricle 55, inferior vena cava 52, superior vena cava 53, and/or tricuspid valve 57. Optionally, left ventricle 42, and/or left atrium 49 are position mapped additionally/instead; for example for performing a mitral valve annuloplasty, for example as described in relation to FIGS. 3A-3B.

The position map of block 110, in some embodiments, provides a kind of “scaffold”, to the positions of which other measurement data (for example, measurement data as described in relation to blocks 101, 114, and/or 116) may be associated (the operation of association is described further, for example, in relation to block 118).

### Localization of Vulnerable Structures

At block 101, in some embodiments, anatomical structures of special concern for damage during a procedure are located and/or tagged. These may also be considered areas to avoid during implantation. A primary target in annuloplasty is the valve itself, and more particularly, for purposes of attachment, the fibrous tissue area of the valve annulus. Characterization of the valve is detailed further in relation to block 114. However, this target is potentially in proximity to one or more areas with particular vulnerabilities, and it is a potential advantage to know clearly where an annuloplasty device is relative to these structures.

As examples, these areas with particular vulnerabilities include, in some embodiments, the AV node 60A, the bundle of His 60, and the right branch of the coronary artery 59. Damage to the AV node 60A and/or bundle of His 60 can result in complete or partial block (e.g., AV block), which can occur in different degrees of severity, up to and including cardiac arrest and possible death. Both of these structures are near enough to the tricuspid valve that a significant risk of complication exists: an annuloplasty implant fastener could cause acute damage, and/or pressure from the annuloplasty device 112 itself (and/or fastener) might eventually induce damage and resulting block of cardiac impulse transmission. Puncture of the coronary artery 59 is another severe complication; bleeding and loss of heart perfusion may require unplanned interventions to manage, and death is again a potential outcome.

Accordingly, it is a potential advantage to locate these structures by whatever means are available, and then, in some embodiments, continue to track their location relative to ongoing procedure activities, to help ensure that damage does not occur, and/or to guide earlier activities so that the procedure does not later on reach an impasse, or require extraordinary measures to avoid causing damage to sensitive heart areas. In some embodiments, structures of the electrical conduction system of the heart (such as the bundle of His or the AV node) are identified by the relative time of waveform arrival to their location, compared, e.g., to surrounding tissue, and/or a reference location such as the sinoatrial node.

In some embodiments, the position of the coronary artery 59 is “tagged” by inserting a catheter wire 111 into it, e.g., via access from the coronary artery 59. In FIG. 2B coronary artery 59 wraps around an exterior portion of the anterior wall of the right atrium and ventricle which is shown cut away in the drawings) Catheter wire 111 may comprise, for example, an electrical wire, or another thin, longitudinally extended probe, such as an electrode microcatheter. The catheter wire 111 can be driven with any suitable pattern of electrical and/or magnetic activation. Additionally or alternatively, wire 111 is acoustically driven (i.e., vibrated). The resulting signal transmits across the tissue barrier(s) separating the lumen of the coronary artery from the lumen of the right atrium and/or ventricle. Locations where the activation pattern is sensed strongly (e.g., by an electrode probe 102, and/or a suitable acoustic transducer) are then recorded as being close to the coronary artery 59 (optionally with a distance decreasing with correspondingly increasing sensed amplitude). It should be understood that the method is optionally carried out with a catheter probe comprising a plurality of separate electrodes (or other transmitters, for example, acoustic or magnetic transmitters) in lieu of a single wire. The transmitters are optionally driven all together (by the same potential), or separately; e.g., with separately distinguishable frequencies and/or periods of transmission.

Locations within the right atrium 51 and/or right ventricle 55 where the signal is strongest (largest amplitude) represent locations nearer to the coronary artery 59 which are preferably avoided by potentially traumatic implantation activities like fastener attachment. The example of a fastener is used herein, but it should be understood that any object (herein, a “potentially traumatizing device”) which has the potential to transmit harm by penetration, pressure, heating, or other trauma, and can be tracked, is optionally monitored and/or guided as is described for the example of a fastener. Examples of such devices include RF ablation probes, needles, cryoablation probes, and implanted devices of any type which exert potentially harmful pressure onto tissue when implanted. In some embodiments, positions within some specification of estimated distance and/or of signal amplitude are considered as being in hazardous proximity to the coronary artery 59; and optionally an indication is provided to the user (e.g., a visual, auditory, and/or haptic alert) that the zone of hazardous proximity has been entered. The zone may be determined by a distance/signal amplitude threshold, and/or determined according to additional considerations such as the type and/or estimated orientation of a tracked device (e.g., the direction of a pointed end of the device) that is approaching the zone of hazardous proximity. In particular, fasteners which operate as anchors by penetration of tissue may be tracked (e.g, configured to be used as electrodes making measurements from which position can be estimated absolutely and/or distance relative to the zone of hazardous proximity can be estimated), and monitored for entry into a zone of hazardous proximity.

It should be understood that distances may be measured between any combination of two adjacent body lumens, e.g., body lumens which are both blood-filled (heart chambers and/or blood vessels). In particular, a transmitting catheter wire or other long transmitting probe placed within a longitudinally extended and radially narrow lumen (e.g. a blood vessel) provides a potential advantage insofar as the transmitter can be operated as a single unit to “tag” a long longitudinal extent of the lumen.

In embodiments wherein transmission is distinguishable among a plurality of transmitters distributed along a catheter probe, proximity is optionally determined per segment defined by each transmitter—creating a segmented proximity warning zone (segmented specification of the zone of hazardous proximity). In some embodiments, segmenting information is used to allow establishing different avoidance thresholds for different segments. For example, a segment which is more “padded” by overlying tissue of a separating wall might appear to be distant from the lumen, even though, upon insertion of a fastener in that region, there may still be a danger of trauma. In another example, a particular segment may be considered “safer” (shorter safe approach distance), e.g., because it is less likely that a fastener will be oriented in a direction which threatens it. In some embodiments, a catheter probe bends around the lumen into which it is transmitting, potentially creating non-uniformities in the spatial distribution of it signal amplitude. A segmented probe provides a potential advantage by allowing location to be more precisely identified (e.g., the contribution of more distant segments can be ignored as irrelevant to proximity detection).

Optionally, these locations are determined at an early part of the procedure, and then catheter wire 111 electrical activation is discontinued (and the catheter wire 111 optionally withdrawn). Later on, in some embodiments, an operator is warned of proximity (for example, of fasteners introduced to the area), e.g., via a displayed image (optionally, a live-updating image). Alternatively, the catheter wire 111 remains active and in place. An approaching electrode (optionally including electrically conductive device parts, which may include the fasteners) will then, in some embodiments, sense the coronary artery “warning signal” regardless of whether the relative fastener and coronary spatial positions are explicitly known. Optionally, both position mapping and proximity sensing of the coronary artery 59 are used, potentially increasing safety and/or reliability. Note that in FIGS. 2E-2G, illustration of coronary artery 59 and catheter wire 111 is suppressed for reasons of drawing clarity, but it is optionally present.

Additionally or alternatively, in some embodiments, coronary artery 59 is monitored for inadvertent punctures or near-punctures. A device (such as a fastener, e.g., a screw) which is being attached to tissue is configured to act, from its tissue-penetrating tip, as an electrode. Upon accidental penetration and/or near-penetration of coronary artery 59, a measured impedance between the device tip and a catheter wire 111 or other electrode inserted into the coronary artery will display a sudden drop. The sudden drop is an indication of a potential injury to the coronary artery (a risk of fastener penetration), and/or is detected automatically and a warning indication of the potential injury is produced. Early warning potentially leads to a reduction in the severity of accidental penetrations by allowing them to be stopped before they are made worse, and/or allowing beginning mitigation actions immediately.

In some embodiments, the spatial extent of the coronary artery 59 is mapped using measurements made by an electrode probe during insertion of the electrode probe into the coronary artery 59. The electrode probe, in some embodiments, comprises a plurality of electrodes. Different electrical fields (e.g., alternating at different radio frequencies) are induced between different sets of other electrodes; for example, body surface electrodes or electrodes on a catheter inserted into the coronary sinus. These electrical fields “tag” space through the region of the heart, including the coronary artery 59. By a process of computational reconstruction (for example, as described in WO 2019/034944 A1, entitled RECONSTRUCTION OF AN ANATOMICAL STRUCTURE FROM INTRABODY MEASUREMENTS), measurements of these “tags” at different locations can be converted from electrical measurements (e.g., of voltage and/or impedance) to measurements of spatial position. This may use constraints such as knowledge of inter-electrode distances, and assumptions about the continuity of electrical field properties as a function of position. In effect, knowing inter-electrode distances gives a measure of the mV/mm scaling at each measurement position. Constraints of electrical field continuity (alternatively described as assuming that relatively similar measurements occurred at correspondingly nearer locations) allow reconstruction to exclude solutions which meet the scaling constraint, but are too “jumpy” to be likely, or even physically plausible.

Optionally, measurements from coronary artery 59 are treated as being part of a larger set of measurements including measurements from larger regions (and from other electrode-carrying probes), for example, measurements made by a catheter-borne electrode probe moving within one or more lumens of the adjacent heart. Insofar as two or more measurement probes measure the same electrical fields (even though separated by solid tissue barriers such as heart and/or blood vessel walls), the process of reconstruction of space from a cloud of voltage measurements can use the electrical measurements of all such probes to reconstruct a common spatial model of the heart regions in which they move. The spatial positions assigned to measurements from the probe which was in the coronary artery are, accordingly, assigned to be positions of the coronary artery.

Accordingly, in some embodiments, movements of a probe in the right atrium/right ventricle (for example; it should be understood that these body cavities serve as examples of body cavities more generally) are directly assessed for their proximity to the right coronary artery (for example; it should be understood that other blood vessels are additionally or optionally mapped using this method), the position of which is modeled in the same spatial coordinate system as the space which is directly accessible to the probe. Optionally, measurements from different probes are adjusted as necessary to account for differences in electrical measurement offsets and/or gains; for example, using predetermined calibration values, and/or calibration values determined on the fly (e.g., by comparing measurements at positions known from other considerations such as limits of motion and/or landmarks to be identical and/or adjacent). It is noted in particular that inter-electrode distances used for local scaling determinations need not be the same in both electrode probes, or even the same among all electrodes.

In some embodiments, the location of coronary artery 59 is determined by another method: for example, flow of blood through the coronary artery 59 may return a Doppler signal under ultrasound imaging, or the particular dielectric properties of blood may induce an electrically sensed impedance signal for a sensing electrode approaching a portion of coronary wall behind which the coronary artery 59 lies.

In the case of electrically active structures specialized for impulse transmission, there is produced a characteristic pattern of activity which is sensed, in some embodiments of the present disclosure, by a suitably configured electrode probe when it approaches and/or contacts the structure and/or a cardiac wall within which the structure is embedded. Electrical activity of the AV node 60A and/or bundle of His 60 is optionally characterized, for example, by timing of the electrical activity relative to the body-surface recorded ECG, or by the time course of single- or dual-electrode recorded electrical activity (e.g., rise time, fall time, and/or baseline-to-baseline time).

In some embodiments, artificial stimulation (pacing) is used as part of locating electrically active structures, e.g., by noting locations from which pacing is effectively entrained, and/or by correlating measurements of electrical activity away from the pacing electrode with the known time of injection of pacing current from the pacing electrode.

In some embodiments, measurements having patterns of activity characteristic of the AV node 60A or the bundle of His 60 are located within the compound model of block 118; for example, using one of the methods of measurement coordination described hereinabove. In some embodiments, location of these structures is optionally performed as part of activities to more generally mapping the electrophysiology of the heart, for example as further described in relation to block 116.

Implantation of an annuloplasty device 112 preferably also avoids unintended interference with other structures and functions of the right atrium 51: for example, avoids blocking inflow from the coronary sinus, and avoids more generally interfering with the electrical conduction system of the heart, including nodes and pathways between the AV node 60A and the SA node.

### Valve Characterization

In some embodiments, electrically and/or electrophysiologically measured data is combined with detailed anatomical information to produce a data structure which is a compound model representing an implantation target in the heart having enough detail to support showing (e.g., on a visual display of the model) where an implantation is occurring, including a distinction between at least two of: targeted tissue (e.g., fibrous tissue for anchoring in), non-targeted tissue (e.g., cardiac muscle adjacent to the fibrous tissue), and vulnerable tissue (e.g., the coronary artery 59, bundle of His 60, and/or the AV node 60A).

To provide the anatomical information, at block 114, in some embodiments, the valve itself (e.g., tricuspid valve 57) is characterized in advance of implantation. It is emphasized that the anatomical information discussed below relates to detailed aspects of valvular anatomy. They include, in particular, shapes of the leaflets and the commissures along which they coapt (or fail to coapt), size of the valve annulus, and optionally dynamics of each. This is information which can be of particular assistance in guiding a valve annuloplasty procedure.

FIG. 4A shows some of the major structures of a tricuspid valve 57, including the three leaflets (posterior leaflet 57A, anterior leaflet 57B, and septal leaflet 57C) and the valve annulus 57D. Also shown are the AV node 60A (which should be avoided during implantation to avoid complications), and the opening to the coronary sinus 61 (inflow from which should not be physically blocked). The valve leaflets 57A, 57B, 57C come together along commissures 57E, 57F, 57G. Also indicated is a position of a portion of a coronary artery 59. What is shown is an idealized structure. Characterization of the tricuspid valve, in some embodiments, comprises obtaining information which provides more patient-specific details of valvular anatomy.

Valvular anatomy can be variable over several parameters that may affect how an annuloplasty procedure is performed. For example, there may actually be from 2-4 distinguishable leaflets (and more may be seen pathologically). The leaflets may be joined together for a longer or shorter distance before they split at their commissure, and the locations of the commissures may be different depending on leaflet number, size, and/or orientation.

A primary reason to perform annuloplasty is to reduce regurgitation consequent to the failure of valve leaflets 57A, 57B, 57C to coapt and form an adequate seal during contraction of the right ventricle 55. Different cases have different pairings of leaflets failing to coapt along their commissures. Failure to coapt may be accompanied by prolapse, where one or more of the leaflets are pushed back into the atrium, instead of correctly coapting.

Poor coaptation may be due to expansion of the valve annulus. Additionally or alternatively, there may also be holes in individual leaflets, shortened leaflets, or another morphological problem; the problem may be congenital and/or acquired. In patients with pre-existing implants, there may be interference with valve leaflet function by implant parts such as pacemaker leads. Poor coaptation potentially relates to function of the chordae and/or papillary muscles of the heart; for example, papillary muscles damaged by ischemia may fail to shorten as usual, potentially contributing to valve leaflet prolapse.

With respect to variations in anatomy and pathology, an important issue for planning is how to attach a valve annuloplasty device so that it operates to good results together with the patient's specific anatomy, consistent with avoiding damage to vulnerable structures, for example, those identified by electrical and/or electrophysiological techniques (e.g., as described in relation to block 101).

When annuloplasty device 112 is cinched (or otherwise actuated), for example, it is preferable, in some embodiments, that the annuloplasty device 112 exert shrinkage upon portions of the valve 57 which are more likely to benefit from this treatment—e.g., annulus shrinkage may be more preferable through a circumferential extent crossing the posterior-anterior commissure, or crossing the anterior-septal commissure. Since the posterior-anterior commissure is circumferentially further from sensitive structures specialized for impulse transmission such as the AV node 60A, the difference in emphasis on where treatment is critical may also relax constraints on the placement of fasteners, potentially making it easier to avoid sensitive structures.

Valve annulus expansion has been found to be greatest on portions of the valve annulus circumference away from the septal wall. Some annuloplasty devices 112 have two free ends (for example as shown in FIG. 6). It may be preferred for a particular procedure to avoid placing these free ends on opposing sides of a commissure—or if the coaptation at the commissure is normal, such placement may, on the contrary, be preferable (for example, to avoid over-closure of the valve 57

Furthermore, the valve 57 by its nature is not a static structure, and may be recorded dynamically as it changes shape over the course of a heartbeat cycle and/or over the course of a respiratory cycle. Functional information, in some embodiments, is extracted from valve behavior over time. The shape of the tricuspid valve 57 during right ventricle 55 contraction (when the tricuspid valve 57 should be closed) is particularly useful as an indication of regurgitation. Additionally or alternatively, the valve's open and/or transitional states can be used in determining aspects of valve anatomy such as commissure position and/or length. Transitional states of valves may also reveal information about the functional state of the valve 57, for example revealed in the by time course of valve leaflet motions and/or leaflet “flutter”. Transitional state motions characteristic of the valve leaflets may also help to identify them, for example, when performing component analysis to isolate cyclic components, e.g., as described in relation to FIG. 7B.

The region of valve annulus 57D itself may show a mix of behaviors as a function of heartbeat phase, e.g., different for muscular parts (which will expand and contract during the heartbeat cycle), and fibrous tissue of the valve annulus 57D itself (which is relatively static, but may still be affected by muscular forces acting on it). The distinction of fibrous and muscular tissue may be difficult to make purely from motion analysis, however; and in some embodiments is confirmed by, determined instead by, or determined jointly with the use of electrophysiological measurements, for example as described in relation to block 116. Tension in the valve annulus 57D is potentially indicated by a degree of flexibility shown during the heartbeat cycle; the tension in turn provides, for example, a potential indication of where tightening of the valve ring is more likely to have intended treatment effects.

During implantation, it is preferable, in some embodiments, to anchor in the fibrous tissue, since it is more stable; and furthermore, there is perceived to be a lower likelihood of inducing functional damage. However, the ring of fibrous tissue can be thin, increasing a need for high-resolution anatomical information and/or electrophysiological confirmation.

The valve annulus 57D may undergo phasic changes as a function of respiratory phase and/or mode of ventilation. Positive pressure ventilation may tend to compress the heart (with corresponding effects on the valve annulus circumference), potentially complicating the annuloplasty decision as to how much cinching is needed so that the valve functions correctly under normal respiration pressures. Normal respiration can also produce phasic changes in heart morphology which potentially include alterations in valve annulus 57D size.

In some embodiments, phasic movements (changes in shape) are accounted for by methods of frequency-correlated component analysis, described, for example, in relation to FIG. 7B.

Methods of obtaining three-dimensional images and/or functional images of heart valves— potentially including resolution of locations of insufficient coaptation—include ultrasound and electrical field-based imaging (measurement) methods, used in some embodiments of the present disclosure. Ultrasound-based methods include transthoracic echocardiography (TTE), intracardiac echocardiography (ICE), transesophageal electrocardiography (TEE), and tissue Doppler echocardiography (TDE). Electrical field-based imaging methods, in some embodiments, comprise moving an electrode probe within a cardiac chamber while making electrical measurements of exogenously produced electrical fields—and reconstructing the shape of the cardiac chamber using the measurements and optionally additional information such as relative spacings of the measurement electrodes, and/or prior knowledge of aspects of the electrical field's spatial distribution. In some embodiments, electrical measurements are made using electrodes placed on either side of the valve 57.

Electrical field-based measurements can also be used to sense regurgitation (for example, in analogy to information on blood flow patterns produced using TDE), by a method comprising injecting and/or tagging fluid to create tracer fluid in a ventricle, and measuring disturbances in magnetic and/or electrical properties caused by retrograde migration of the tracer fluid through a regurgitating valve. The tracer fluid can comprise anything which creates an electrical, dielectric, and/or magnetic contrast; for example by injection (e.g. of saline or another fluid), temperature manipulation (e.g., localized heating of blood), and/or structural manipulation (e.g., using ultrasound to create cavitations in blood). The disturbance measured can be, for example, changed voltage or magnetic field strength measured from an induced electrical field and/or magnetic field, or changed impedance measured by an electrode. Additionally or alternatively, FIG. 2C illustrates the placement of a pigtail catheter 113 in the right ventricle, from which location boluses of saline or another tracer fluid are optionally injected. In cases of regurgitation, the tracer may end up leaking across the heart valve 57 where it affects the electrical environment of the right atrium 51, and sensing, e.g., by multi-electrode probe 102.

In some embodiments, impedance-based valve leaflet mapping is performed. As a valve leaflet moves nearer to an electrode pair, its relatively insulating properties (e.g., compared to blood) lead to an increase in measured impedance between them; as it moves away, impedance drops again. An example of a time course of such a time-varying impedance signal is shown and described, for example, in relation to FIG. 14, herein. A characteristic feature of valve motion, in some embodiments of the present disclosure, is the occurrence of a doubled opening/closing cycle over the course of a single heartbeat cycle. In comparison, motion of connective tissue of the valve annulus itself, for example (e.g., fibrous tissue region comprising annulus 57D of FIG. 4A), displays little or no cycle doubling.

This information may be indicative in some instances of motion signal features. Use of an external synchronization indication, however, is potentially less prone to confounding factors such as a lowered signal-to-noise ratio and/or differences in motion signal waveform at different positions near a moving structure of interest such as valve leaflets.

CT scans and MRI scans are also potential sources of anatomical information. Apart from structural information, magnetic resonance imaging, can optionally provide a measure of regurgitation, for example, based on stroke volume differences between the left and right ventricles.

It should be understood that early measurements indicating valve (and valve leaflet) anatomy may have a relatively low resolution limited by the low number of measurements available. As more measurements are obtained and/or are more computer processing time is applied to producing the compound model, available image detail may correspondingly increase.

### Endogenous Electrophysiology Measurements

At block 116, in some embodiments, endogenous electrophysiology is mapped. Electrophysiological mapping comprises moving an electrode probe within the heart to visit various positions, concurrently with measuring endogenous electrical activity at the visited positions.

In some embodiments of the present disclosure, movement of the electrode within the heart is accompanied by measurements using a second modality. The measurements using the second modality serve as scaffold measurements (indicating spatial locations) for the compound model, and/or supplement the electrophysiological mapping measurements in generating the scaffolding of the compound model. For example, the same electrode probe may also be used to make measurements of exogenously generated electrical fields extending through the heart chambers, and the resulting measurement used to generate a position map of the heart. In another example, ultrasound images showing a position of the probe are correlated with electrophysiological measurements made using the same probe.

The electrophysiological measurements, in some embodiments, are presented in image form, mapped to positions defined by the compound model. This can be done in different ways, and optionally more than one within a single image. In some embodiments, for example, color coding is used to distinguish times at which the heartbeat impulse reaches different areas, earlier or later in the heartbeat cycle. In some embodiments, waveforms with different characteristics (e.g., relative amplitudes of components) are assigned to different categories, and shown differently in the image in accordance with category—for example, areas with waveforms characteristic of proximity to structures such as the AV node and the bundle of His are tagged by a visual marker, and/or shown differently (e.g., a different color, brightness, saturation, transparency, and/or texture) than areas with waveforms characteristic of cardiac tissue alone.

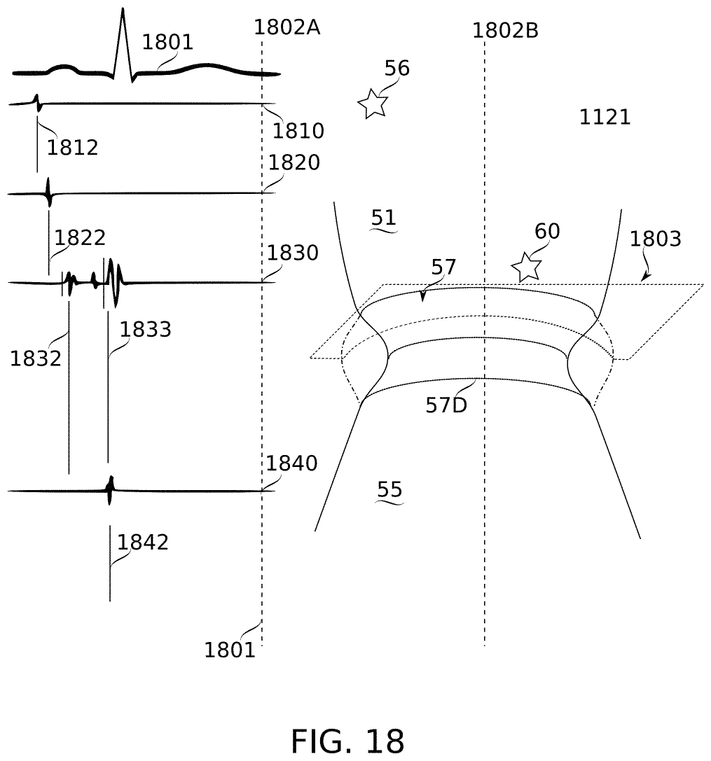

Brief reference is now made to FIG. 18, which schematically illustrates another method of estimating the position of the plane of the valve annulus (the AV plane), according to some embodiments of the present disclosure.

FIG. 18 schematically represents (in cross-section) anatomy around the AV plane 1803, including (above AV plane 1803) the right atrium 51, and (below AV plane 1803), the right ventricle 55. Sinus node 56 and bundle of His 60 are also indicated. The AV plane passes through heart valve 57, including through valve annulus 57D. AV axis 1802B (shown for reference and “imaginary”, not indicating a physical feature in its own right) extends substantially perpendicular to AV plane 1803, and is also represented on the left as imaginary AV axis 1802A, to indicate relative positions at which intracardiac electrograms (IEGMs) 1810, 1820, 1830, and 1840 are recorded. ECG 1801 shows the body surface electrocardiogram. Time markers 1812, 1822, 1832, 1833, and 1842 indicate times of selected features shown in the different IEGMS 1810, 1820, 1830 (two marks), and 1840, respectively. In particular, time markers 1812, 1822, 1832 show P-wave spikes, and time markers 1833, 1842 show QRS-wave spikes. These features are most prominent when measurements are made by electrodes in contact with the heart wall, and substantially reduced (or absent) when measured from deeper within the right atrial lumen.

In some embodiments, for each pair-position of electrodes used to collect IEGM signals, a code is assigned based on features seen in the corresponding IEGM. For example:

TABLE 1

| code | region | note |
|---|---|---|
| 0 | Lumen | not in contact with a lumenal wall |
| 1 | V | contact with a ventricular wall (QRS wave spike) |
| 10 | A | contact with an atrial wall (P wave spike) |
| 11 | AV | contact at or near the AV ring (P wave and QRS wave |
| | | spikes both visible). |
| 99 | — | rejected due to noise or other classification problem |
| −1 | — | ignored, e.g., because of the lack of a distinct P-wave. |

The code values are arbitrary, and may be substituted with other values.

Spatial positions of the electrodes while measuring IEGMs are also recorded. This allows associating codes to a plurality of positions, resulting in some A-coded positions (e.g., code 10) above the AV plane, and some V-coded positions (e.g., code 1) below the AV plane.

Optionally, the AV plane is defined as a Euclidean plane that separates A-coded positions from V-coded positions, optionally at an about equal distance from either. The method to determine this position comprise using a classifier, e.g., a support vector machine (SVM) or stochastic gradient descent classifier.

Optionally, measurements from locations used in the determination of the AV plan are grouped (e.g., binned and/or weighted) according to heartbeat phase, and the AV plane position calculated for each of a plurality of different phases of the heartbeat. This may be used to generate a phase-dependent position of the AV plane, which may move to different locations and/or orientations as the structures of the heart wall itself move during the course of the heartbeat cycle.

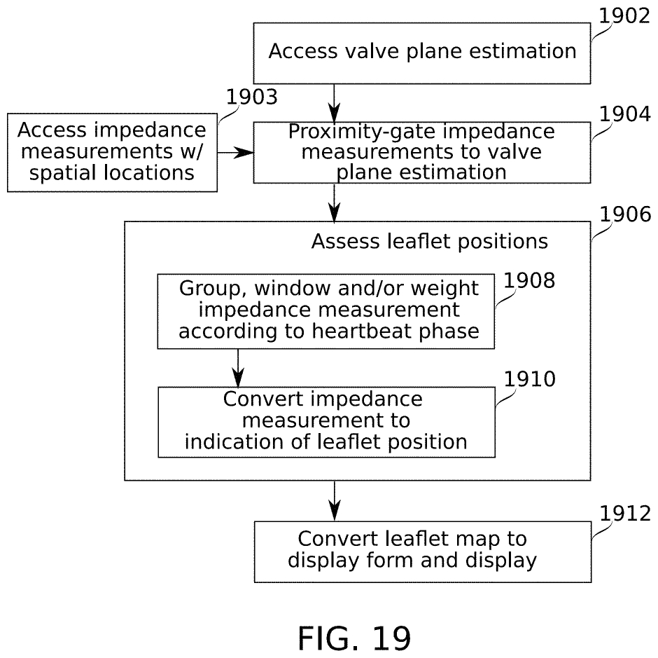

In some embodiments, an AV plane determination is used to create a reference zone which may be used in the locating and/or characterization of structures at or near the AV plane, for example, the valve leaflets, valve annulus, and/or the hinge between the valve leaflets and valve annulus. For example, embodiments in which valve leaflet locations are mapped are described in relation to FIG. 19; embodiments in which hinge regions of the leaflets are mapped are described in relation to FIG. 7A.

Additionally or alternatively, positions which are AV-coded are used to define the AV plane, e.g., the plane which bisects the cloud of recorded AV-coded positions is calculated. This may give somewhat different results, e.g., due to potential difficulties in reaching the edges of the AV-coded zone due to the complex shapes of the heart valve.

Another method of coding, in some embodiments, uses ratios of P wave and QRS wave spike amplitudes seen in the IEGM. In some embodiments, the AV plane and/or a region containing the AV plane is determined by using characteristic quantitative differences in IEGM features at different positions along the AV axis. In some embodiments, a certain ratio of IEGM spike amplitude during the P wave to IEGM spike amplitude during the QRS complex (e.g., a ratio of 1:1.5, 1:2, or another ratio) is assigned as indicating a position at the valve, and “midway” along an imaginary atrioventricular axis. Optionally, positions at the valve are indicated by a range of ratios, for example, 1:(1.5±0.5), 1:(2±0.5), or another range. Locations at which a larger ratio between IEGM spike amplitudes during the P wave and the QRS complex is measured are assigned as “atrial”; locations where a relatively smaller ratio is measured are assigned as “ventricular”.

For example, the P:QRS IEGM spike amplitude ratio in IEGM 1810 is much greater than the ranges given (and thus “atrial”), the P:QRS spike amplitude ratio in IEGM 1840 is much smaller (and thus “ventricular”). In IEGM 1830, the ratio is about 1:2, and thus may be within the selected ratio range which is assigned as being at the position of the valve, for example, 1:2±0.5). Optionally, one particular ratio is assigned as “the” AV plane; optionally it is sufficient to define a certain volume as containing the AV plane, and/or as being “sufficiently close” to the AV plane for the purposes of the structural identification, mapping, or other determination being performed.

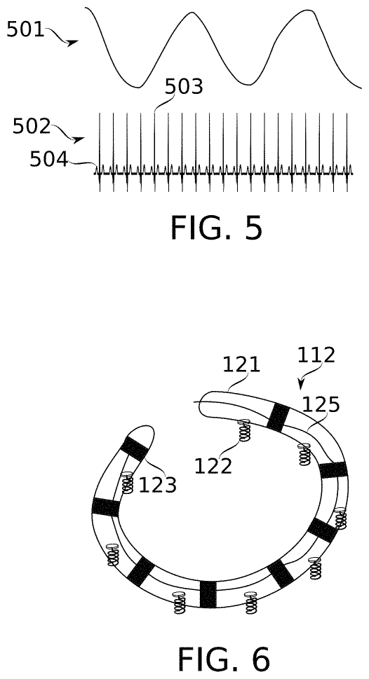

It is noted that the valve annulus comprises connective tissue which presents a barrier to the propagation of waveforms between the atrium and ventricle, helping to sharpen the spatial transition between P wave spike-dominated and QRS complex spike-dominated regions. Optionally, a region through which the transition occurs is identified as comprising positions at the valve. In some embodiments, gradations between two different characteristic waveforms are color coded according to threshold values, and/or shown along a color or other visually displayed gradient. For example, conversion along an atrioventricular axis from a dominant (higher-amplitude) P-wave spike 504 to a dominant QRS complex spike 503 (FIG. 5) is shown by different colors selected from a color look-up table.

By making repeated measurements at different locations (e.g., different radial offsets from an imaginary atrioventricular axis extending through the center of the valve annulus), an orientation of the valve annulus can also potentially be revealed. For example, a plane going through three or more points, each identified as being at the level of the valve annulus may be defined as the valve plane, i.e., a plane which intersects the valve annulus at two opposite sides of the valve annulus circumference, and optionally all around its circumference. When more than three points are available, the plane does not necessarily go through all of them, and may be defined by an error fitting classifier algorithm; for example, one implemented using SVM or stochastic gradient descent.

As for the “coding” method based on the presence or absence of different waveforms (exemplified in Table 1, above), reference positions used in determining the AV plane position need not all be directly measured locations at the valve annulus level. For example, a reference function indicating rates of transition between atrial-side and ventricular-side electrophysiological signals (including shifts in amplitude) as a function of movement along the atrioventricular axis can be measured during a single passage between the two sides. The valve-level positions can then be estimated using the same reference function to interpolate between measurements made above (atrially) and below (ventricularly) the level of the valve annulus. A potential advantage of having an estimate of the orientation of the valve is in planning, tracking and/or evaluating movements of a probe involved in structural heart disease treatment of the annulus as the probe moves around a significant portion (e.g., at least half) of the annulus circumference, e.g., as may be performed during implantation of an annuloplasty device. In some embodiments, the identification of the valve annulus location comprises identifying a plane that is estimated to intersect its full circumference. In case a valve annulus is distorted to a non-planar shape (e.g., saddle-shaped, for example due to a geometric deformity of the valve annulus), measurements at a plurality of locations around the valve annulus may determine this by sensing different atrioventricular axis displacements at different circumferential locations.

In some embodiments, a full IEGM waveform (covering a full heartbeat cycle) is assigned to a single “position” as defined by the scaffolding position map of the compound model. For example, the position may be a time-weighted average of positions measured during the full heartbeat cycle. In some embodiments, measurements of partial waveforms (from a period shorter than a full heartbeat cycle) are assigned to the exact compound model-defined location at which they were measured (with a precision insofar as is known). Optionally, a full waveform for a particular position is constructed using data from neighboring positions. For example, periods of a waveform with missing data for parts of the heartbeat cycle are filled in with information from a nearest neighbor, by (e.g., distance-weighted) averaging of nearby model positions for which measurement data is available, and/or by another method of interpolation. Additionally or alternatively, interpolation of missing full cycle data may also be performed between times for which data is available. In some embodiments, interpolation is jointly performed over time and space; e.g., the shape of the nearest available (in position space) waveform portion for a period of a heartbeat cycle is amplitude-scaled to fit the available measurements at a particular position which overlap in time.

As described also for the coded-position method of AV plane determination, measurements from locations used in the determination of the AV plan are optionally grouped (e.g., binned and/or weighted) according to heartbeat phase, allowing estimation of the dynamic (heartbeat phase dependent) position of the AV plane.

Information acquired by electrophysiological mapping measurements, in some embodiments, has been described in part with reference to block 101 (localization of structures such as the bundle of His 60 and the AV node 60A), and to block 114 (electrophysiological distinguishing of fibrous valve annulus tissue and myocardial tissue). Those electrophysiological mapping operations may also be considered to fall within the scope of the operations of block 116.

In some embodiments, electrophysiological mapping comprises measuring differences in electrophysiological properties in a way that correlates electrophysiological signals with spatial position along one or more directions (e.g., along an imaginary atrioventricular axis), and/or distinguishes different positions as being, for example, atrial, within the valve (atrioventricular), or ventricular. For example, atrially-measured IEGM tends to have a relatively high amplitude at times corresponding to the P-wave 504 of normal externally recorded ECG. Ventricularly measured electrical activity corresponds to a higher amplitude at the time of the QRS complex 503. Positions in between (at the level of the valve itself) tend to be intermediate in character. They may also show waveforms corresponding to other signals, for example, at locations in proximity to the bundle of His 60.

Endogenous electrophysiological signals propagate with characteristic propagation velocities, so that changing latency may also serve as a marker of probe position. Moreover, different components of endogenous electrical activity may have different conduction velocities; for example, signals propagate faster through the AV node and bundle of His than through other myocardial cells. Optionally, differences between the phase (optionally measured, for example, by time-to-onset, time-to-peak, or another metric) of two different electrical signal components provide an indication of probe position—for example, an electrical impulse propagating generally in a ventricular direction along an atrioventricular axis arrives at an atrial position before arriving at a ventricular position or a valvular position.

Additionally or alternatively, electrophysiological mapping potentially distinguishes between types of tissue: myocardial or fibrous, for example; and/or locations of impulse transmission-specialized structures such as the AV node 60A and the bundle of His 60. Fibrous tissue is non-contractile, so electrical signals measured while in contact with it are, for example, dampened compared to nearby myocardial tissue. This is used, in some embodiments, to assist in identification of the bounds of fibrous tissue in the valve annulus.

### Integration of Information in a Compound Model

Continuing with the description of FIG. 1: at block 118, in some embodiments, measurement data from any of blocks 110, 101, 114, and 116 is integrated into a compound model.

In some embodiments, the position map of block 110 is used as the scaffold for the compound model. In embodiments where it is used as the model's “scaffold”, the position map provides a unifying frame of reference, to and/or through which the measurements of other measurement modalities are related as being, for example “closer” or “further” from one another. This may be useful during a procedure, for example to assist in finding, approaching and/or avoiding certain targets.

In the case of a position map, distances can be expressed in terms of physical space (spatial distance), but other types of distances (examples are mentioned below) may also serve for purposes of procedure guidance and/or monitoring.

Once association to position map locations is established for a measurement (different methods of doing so are described, for example, in relation to blocks 101, 114, and/or 116), it is integrated, in some embodiments, into a compound model of heart function and anatomy which can be used for display, for providing procedure guidance, and/or for monitoring one or more aspects of procedure status. In some embodiments, the display shows both the compound model of the heart and the estimated locations within the model of equipment introduced to the heart; for example, positions of measurement probes and/or the annuloplasty device.

It should be understood that the compound model, in some embodiments, comprises a data structure which models the heart, and is not necessarily a visible production such as an image for display (though it may, in some embodiments, be an image and/or be used to generate an image; including, in some embodiments, a live-updated image that shows changes in positions and/or shapes of heart structures and/or equipment introduced to the heart as a procedure is carried out).

The data structure is not necessarily representative of physical space (for example, it may be representative of a “measurement space”), though representation of physical space is a feature of some embodiments of the present disclosure. It is convenient and, in some embodiments, preferable for the “scaffolding” which unifies data of different types to be expressed in terms of spatial position; for example, since this is readily represented and understood by a surgeon performing a procedure. However, e.g., if the operations of block 110 are omitted, then the “scaffolding” provided by a position map is optionally provided instead by combining data from one or more of the operations of blocks 112, 114, and/or 116, and may not include spatial position data as such—but rather represent a kind of “functional topography”, wherein distortion of absolute distances is allowed, and procedure-relevant characteristics of the mapped region are more heavily emphasized by features of the display (such characteristics optionally include, for example: “on the fibrous tissue of the valve annulus”, and/or “dangerously close to the bundle of His”).

The resolution of the compound model (and/or images generated therefrom), in some embodiments, is adaptive to the available data. For example, a “rolling ball” algorithm is used, in some embodiments to generate a connected surface from a cloud of measurement positions. The algorithm behaves, in some embodiments, as if a ball of a certain diameter is brought from outside the cloud into contact with it, as close to the cloud center as contact with the cloud measurement positions permits. Optionally, the algorithm includes refinements such as a noise-reducing “elasticity” parameter allowing the ball to penetrate a short distance past single measurement positions, but for a shorter distance past a plurality of measurement positions. In some embodiments, the parameters of the rolling ball are changed (e.g., the diameter is decreased) as more measurements become available, and optionally changed differently for different parts of the measurement cloud according to measurement density.

A principle which may be used in some embodiments of the present disclosure to enable combination of data from different measurement modalities to a compound model is that of coordinated measurement: measurements made in different modalities which can be determined to have been made “at the same location” may thereby be integrated into a single compound model as belonging to a same location. Measurements at known offsets in time or space may similarly be related to one another and integrated into a compound model at different locations with corresponding offsets. Moreover, coordinated measurements—e.g., because they occur close in time and/or space—may provide anchoring to other measurements of the same modality (even measurements not directly coordinated with measurements of another modality), allowing the compound model to be formed with greater detail.

For example: while an electrode is used to map endogenous electrophysiology (discussed further in relation to block 116), 2-D X-ray or ultrasound imaging (e.g., as mentioned in relation to block 114) may be used to note the probe's relative position (at least in part) at the moment of some or all electrophysiology measurements. For example, position may be measured in one plane, or otherwise constrained, even if not fully determined. While this information may be insufficient to reconstruct a surface or volume suitable for a position map, the coordinated measurements may nevertheless serve, in effect, to allow partial relative positions (e.g., coordinates within a projection plane) to be determined. In some embodiments, this plane serves as “scaffolding”, allowing at least partial determinations of relative distances along certain spatial directions.

In some embodiments, none of the coordinated data measurement methods measures spatial position as such. For example, locations of anatomical structures of special concern (described in relation to block 101), are optionally characterized, e.g., by their electrical impedance, and/or sensitivity to exogenous manipulations such as fluid injection or pacing. Similarly, endogenous electrophysiology activity measurements may be characterized, at particular locations, by a characteristic linear or non-linear combination of IEGM spike components such as those occurring during the P wave 504, QRS wave 503, and/or features associated with localized structures such as the AV node 60A (buried within the septal wall) and/or the bundle of His 60. Though neither of these measurements is spatial in nature, they can still be used to distinguish locations at which different measurements are made.

It can, furthermore, be determined by one or more of various means (according to the particular embodiment), that two measurements in two different measurement modalities (e.g., impedance and electrophysiological measurements) are coordinate: for example because they are measured using the same probe at the same time (optionally with the same or different electrodes/sensors), because they are measured in same observed positions under imaging by a third modality (whether or not that position is itself characterized as having particular spatial coordinates), because they are measured by two probes in physical contact or otherwise physically constrained to be near one another, or by use of another constraint.

Where coordination of two measurement modalities is not inherent and/or constant, there may still, in some embodiments, be available a limited set of measurements that can be considered coordinate; e.g., because they are known to have been made at the limit of movements of a catheter probe, during travel along similar paths, or for another reason.

Furthermore, for purposes of setting up non-spatial coordination between different measurement modalities, “distances” may be calculated as existing along one or more non-spatial axes related to measurement characteristics—for example, signal phase, amplitude, or one or more eigenvalue vector components. Insofar as measurements of physical values tend to change continuously over time and/or space, measurement distances may be used as information to help establish that measurements are coordinate, and from this a corresponding compound model incorporating those measurements may be established.

Non-spatial metric distances may optionally be used as part of guidance and/or monitoring. For example, in some embodiments, a certain physical distance constraint such as “no fasteners placed within 4 mm of the AV node” is to be satisfied. This constraint is optionally considered to be functionally met by a non-spatial constraint related to one or more non-spatial measurements. For example, in an electrophysiological signal, a relative amplitude and/or timing of a wave component may be required to be uncharacteristic of locations nearby the AV node 60A: for example, peaking at least 3, 5, 10 or more msec too late, and/or at least 10, 20, 30 or more mV different in amplitude.

### Planning and Tracking of the Implantation Procedure

At block 120, in some embodiments, the implantation procedure is performed, comprising within it stages of final planning and implantation itself.

### Multimodal Measurement-Revealed Contraindications

Optionally, the annuloplasty procedure is abandoned at this stage, due to a discovery of one or more contraindications. For example, it may be determined during a procedure that the valve leaflets themselves are damaged or malformed in a way (e.g., with holes, torn, shortened, and/or by fusion with portions of a previously implanted device) that makes the annuloplasty device unlikely to produce beneficial results. In some embodiments, it may be determined, based on inspection of the compound model, that no acceptable surgical solution is available, for example due to proximity of vulnerable structures to the planned location of annuloplasty device implantation, lack of stable tissue for implantation, or another reason.

### Multimodal Measurement-Guided Procedure Planning

Assuming the procedure continues, criteria already outlined above along with additional relevant criteria can now be applied to selecting a specific targeted configuration of the annuloplasty device as it attaches on the valve annulus.

Brief reference is now made to FIGS. 4B-4C, which schematically illustrate examples of displays used in device implantation planning and/or in performing device implantation, according to some embodiments of the present disclosure. FIG. 4B represents an above-the-plane view of a simplified representation of a tricuspid valve, while FIG. 4C represents a cutaway transverse view of a simplified representation of a tricuspid valve.

In some embodiments, a hinge of the valve (that is, the approximately circumferential region wherein the valve annulus ring gives way to the valve leaflets) is identified by any suitable mix of manual and automatic identification methods. Identification of tricuspid valve features including the valve's hinge is described, for example, in relation to FIG. 7A. The identification is optionally completely automatic. For example, any one or more of hinge “cliff” geometry, position along an atrioventricular axis and local impedance values are used and/or combined to identify the internal boundary of the valve annulus ring. Optionally, any one or more of these indications are shown to a user, and the user identifies the hinge location. Optionally, an automatic determination is shown to a user and the user is allowed to correct the automatic determination.

In FIGS. 4B-4C, path 403 represents an estimated circumferential location of a valve annulus hinge; drawn by a user and/or automatically determined. Optionally, locations of other structures are identified in the views of FIGS. 4B-4C. for example, optional markers 404, 405 represent approximate positions of an opening into the coronary sinus and the bundle of His, respectively. An estimated position of the right coronary artery is shown along path 401 (FIG. 4B) and/or a spatial representation of the coronary artery 401A (FIG. 4C). Path 402 represents a planned path along which an annuloplasty device is to be implanted. In some embodiments, path 402 is automatically calculated, taking into account the estimated positions of the coronary artery and the hinge of the valve annulus ring, e.g., expanded radially outward about 3 mm from path 403; optionally adjusted to maintain a minimal clearance from the estimated position of the right coronary artery; e.g., a clearance of 3 mm.

The two views of FIGS. 4B-4C are optionally shown simultaneously or alternately. FIG. 4B is optionally shown during planning, in particular, to allow a clear view of structures along which the annuloplasty device is to extend. The transverse view of FIG. 4C also has potential advantages during device implantation, for example as described hereinbelow.

An annuloplasty device is selected to match the shape and size of the patient's tricuspid valve, and/or the nature of tricuspid valve coaptation problems found; as measured, for example, in block 114. In some embodiments, an annuloplasty device which is otherwise-than-optimal for the size/disease is optionally selected in view of safety criteria (i.e., to ensure that vulnerable structures are avoided); for example to provide extra security that the device can be secured into place without damaging a vulnerable structure.

For procedures where the annuloplasty treatment target is to shrink the valve annulus, a target final size of the valve annulus is also selected. In some embodiments, the target size is simply selected to restore a valve diameter, circumference, and/or radius which is considered standard for healthy patients of the current patient's, e.g., size and/or age. Optionally, this is adjusted for the state of the valves themselves; for example, a valve may receive extra tightening for a patient with a particularly large regurgitating aperture, or a clear propensity for valve prolapse. It should be verified that a resizing annuloplasty device can be shrunk (e.g., cinched) enough to reach the target diameter. Alternatively or additionally, an annuloplasty device is provided for bracing; i.e., stiffening of the valve annulus, which potentially helps to enhance coaptation and/or resist deformations which can lead to valve regurgitation.

Optionally, sizes are selected while compensating for the physiological conditions of the implantation procedure. For example, if positive pressure ventilation is used, the valve annulus may appear smaller than it normally is. Insofar as a certain amount of elasticity may be retained even after shrinking of the annuloplasty device, it may be that the appropriate intervention should involve somewhat more tightening than the level of regurgitation measured under ventilation conditions indicates.

In some embodiments, planning also includes selection of a starting point of the implantation, and selection of the placement of fasteners. This potentially involves finding an implantation solution which simultaneously satisfies a number of criteria:
Other things being equal, the annuloplasty device itself should span the leaflets and their commissures in a way that is best-suited to close up gaps, and without tightening being misdirected to leaflet regions that might not benefit (this is discussed, for example, in relation to block 114).
Fasteners (e.g., screws) are preferably placed, in some embodiments, roughly centered on either side of commissures to be tightened, rather than at the radial position of the commissure itself.
Ends of the annuloplasty device (for an open-loop design) may preferably be placed near edges of the septal leaflet, limiting the span of the device which extends along the septal wall, where some vulnerable structures like the bundle of His and AV node are (the septal circumference is also said to be the segment of the valve circumference which is often in least need of tightening).
The fasteners of the annuloplasty device should be placed close enough together to properly secure the device, e.g., without loose ends, without a tendency to form arches/loops between fasteners, and circumferentially close enough to each other to avoid drawing the annuloplasty device across the valve aperture when tightened, partially blocking it.
The fasteners, however, should not be placed where there is a significant risk of damaging a vulnerable structure, for example the AV node, bundle of His, and/or the coronary artery 59. Optionally, fastener spacing is adjusted wider or narrower to increase the available margin of error with respect to vulnerable structures. Optionally, fasteners are planned to be inserted at irregular intervals to increase the available margin of error at specific locations.
Variability associated with fastener positioning is optionally accounted for. For fastening operations entailing a higher risk of variability, it is optionally preferred to plan to avoid by a greater distance locations of vulnerable structures. For example, the first fastener is potentially more prone to positioning error, since it cannot depend on any previous fastener to help control slippage. It may optionally be planned to place fasteners out of order along the circumference of the annuloplasty implant, potentially transferring variability away from a certain location of particular risk.

In some embodiments, a model of an annuloplasty device selected for use (or a candidate for use) is placed within the context of the compound model (together with its fasteners), and the placement evaluated for how well it satisfies a plurality of different criteria. For example, at least one criterion of safety (e.g., margin of distance of fasteners from vulnerable structures) is evaluated, and at least one criterion of functional efficacy (e.g., anticipated degree of post-operative regurgitation) is evaluated. In some embodiments, the criteria are automatically evaluated for a number of different arrangements of the annuloplasty device and its fasteners (the tested arrangements themselves may also be automatically selected, for example by systematically and/or randomly adjusting positions of device portions). In some embodiments, evaluation is carried out automatically at least in part: for example, distances between fasteners and vulnerable structures are measured automatically using distances represented the compound model/annuloplasty device model; and/or post-operative regurgitation is estimated by shortening a modeled circumference of the valve while assuming valve leaflets retain their already-measured area within that shortened circumference. Optionally, evaluation uses a heuristic, for example, an estimate of risk and/or benefit based on statistically correlated outcomes of past procedures with similar characteristics.

In some embodiments, one or more of the most optimal of the arrangements evaluated is presented (e.g., in a projection or other display of a 3-D view) to an operator for review, manual adjustment, and/or final selection.

### Multimodal Measurement-Guided Annuloplasty

FIG. 2D shows multi-electrode probe 102 retracted to hover above valve 57, from which location it is used, in some embodiments, to monitor (electrically image) the valve during the implantation procedure. Additionally or alternatively, another imaging monitoring probe is used, for example as described for positioning measurements in relation to block 110.

FIG. 2E shows annuloplasty device 112 at the beginning of the implantation procedure, having been advanced into the right atrium over catheter 112A, partially extended from catheter 112A, and partially attached by (e.g., two) fasteners 122. In some embodiments, fasteners are attached by use of a fastener-attached control member 124 operating through catheter 112A and from inside sleeve 121, for example as shown in FIG. 3B (which shows implantation of a mitral valve annuloplasty device using the same working principle as described for the tricuspid valve annuloplasty device 112).

Placement of fasteners, in some embodiments, is guided at least in part by data acquired using the fastener 122 (and/or a position-associated structure such as the fastener's placement control member 124 and/or delivering catheter sheath 112A) itself as an active and/or sensing element. In some embodiments, fastener 122 comprises a metal portion, configured for use as an electrode, for example by attachment to control member 124 (which may itself be conductive), and via that attachment attached to an electrical measurement device (e.g., a voltmeter outside the body).

Herein, a “position-associated” element to, e.g., a fastener 122 is an element (comprising a radiopaque marker, for example, and/or an electrode) which, while not part of the fastener 122 itself, is nevertheless linked to it by a regular and/or predictable offset in position, so that knowing the position of the position-associated element allows estimation of the position of the fastener 122 itself. Optionally, the position estimate uses further information, such as a relative distance of advance into the heart of the position-associated element and the fastener 122.

There are several types of measurements (each comprising a different measurement modality) which may be relevant to position finding, optionally used together in any suitable combination. Examples include the following.

In some embodiments, voltages sensed through fastener 122 (and/or, optionally, a position-associated electrode; for example, an electrode of a catheter sheath 112A used to deliver fastener 122, and/or an electrode of control member 124) are matched to positions in the position map of block 110, through being measurements of same voltage fields which were used in measurements that generated that position map in the first place.

In some embodiments, placement of fastener 122, e.g., along an atrioventricular axis, is guided by measuring endogenous electrical signals using fastener 122 (or another position-associated electrode) as an electrode. For example, the targeted position of the fastener comprises a position at which endogenous electrical signals match an atrioventricular waveform (preferably while avoiding a waveform that also shows evidence of the AV node being nearby). Optionally, a graphical (e.g., 3-D) representation of fastener 122 is dynamically adjusted to represent its position along an atrioventricular axis, for example, presented with a color or other surface characteristic indicative of “atrial”, “ventricular” and or “valve annulus” positioning of the fastener 122.

In some embodiments, depth of insertion of a fastener 122 is determined by measuring how voltage, current and/or impedance sensed from it (and/or injected through it) changes as more of it becomes embedded in tissue.

In some embodiments, an angle of fastener 122 is determined, e.g., by making electrical measurement concurrently with injecting current through it. Electrodes located, e.g., at the position of multi-electrode catheter probe 102 in FIG. 2D “see” a different disturbance in voltage depending on the direction in which fastener 122 is pointed. It may be noted that a preferred direction of insertion of fastener 122, in some embodiments, is pointed directly away from the position of multi-electrode catheter probe 102, an orientation at which fastener 122 will potentially appear most “point like”. At a perpendicular orientation, for example, fastener 122 potentially appears as a more laterally-extended current source from the perspective of probe 102.

In some embodiments, placement of a fastener 122 in an intended tissue type is determined, e.g., by confirming (optionally, before placement is finalized by attachment) that impedance measurements match the expected impedance of the targeted tissue, and/or confirming that endogenously produced electrical signals from the heart measured from the fastener 122 (and/or another fastener position-associated electrode) do not indicate placement within myocardial tissue, and/or do not indicate insertion to a vulnerable location such as the AV node. Optionally, pacing signals are transmitted from fastener 122, e.g., to help confirm that it is not (because of a failure of pacing entrainment) in the vicinity of a vulnerable structure. Optionally, pacing signals are delivered from another heart location, and relative timing of pacing signal delivery and sensing from fastener 122 (or position-associated electrode) used to confirm an intended location of fastener 122, e.g., based on the latency and/or signal strength.

It is noted that the electrical connection between control member 124 and an external electrical signal measuring device may itself form a transducer; e.g., as control member 124 rotates fastener 122, control member 124 may also, in some embodiments, rotate within the grip of a clip (e.g., an alligator clip) that attaches it to the signal measurement device. This may result in small changes to contact quality in a pattern that tracks the rotation, allowing estimation of insertion depth of a screw-type (rotatingly inserted, whether with an external thread, formed as a coil, or otherwise shaped for helical advancing) fastener.

In some embodiments, fastener 122 is used as a transmitting device. In some embodiments, the transmitting is electrical, and sensed, e.g., by multi-electrode catheter probe 102. In some embodiments, fastener 122 is vibrated (e.g., using a vibration crystal, optionally in contact with fastener 122, and/or indirectly through vibration of control member 124), and the transmitted vibrations sensed (e.g., using an ultrasound sensor) to determine one or more aspects of placing the fastener—e.g., distance from one or more sensors (determined, for example, from the relative phase of vibration excitation and vibration sensing), and/or contact with tissue (determined, for example, by vibration damping and/or transmission of vibration through an alternative pathway).

In some embodiments, an imaging modality such as X-ray and/or ultrasound is used, constantly or intermittently, to help register and/or confirm the locations of measurements made in other measurement modalities.

Optionally, prior constraints on the size, shape, and/or position of the fastener are used to limit interpretation of location based on multimodal measurements—for example, if the implantation procedure has begun, then the anchored portion of the annuloplasty device 112 is optionally assumed to limit plausible fastener 122 locations to be between the catheter 112A and the last-implanted fastener 122. In some embodiments, a position-associated electrode (or other marker, e.g. a radiopaque marker when X-ray imaging is used) located on catheter 112A allows its position to be determined. Advance of control member 124 is optionally measured (e.g., via a transducer) past a position of initial exposure of fastener 122 to the internal electrical environment of the heart, and the combination of catheter 112A position and control member 124 advance used to estimate a current location of fastener 122. Optionally, annuloplasty device 112 itself comprises dielectrically distinct structures such as radiopaque markers 123. Effects of radiopaque markers 123 on nearby electrical fields potentially cause measurement changes that indicate, in some embodiments, when a fastener 122 approaches and/or passes a certain radiopaque marker 123.

A potential advantage of using a plurality of measurement modalities for tracking locations of fasteners 122 is to reduce situations of position tracking ambiguity. For example, if one measurement modality alone is consistent with a plurality of different positions, and/or prone to measurement noise that increases uncertainty, conjoining it to a second measurement modality may resolve some ambiguities.

From the perspective of validation (during the implantation phase of the procedure): if successful, placement of a fastener 122 comprises, in some embodiments, satisfaction of a plurality of criteria. It is a potential advantage to demonstrate this by a corresponding plurality of measurement modalities. For example, the available measurement modality data jointly confirm, in some embodiments, any suitable (and optionally changing according to the stage of the procedure) combination of the following specifics, which are given as examples:
The fastener is at targeted spatial coordinates.
The fastener is at a targeted location along an imaginary atrioventricular axis consistent with locations at the level of the valve annulus.
The fastener is not in contact with myocardial tissue.
The fastener is in physical contact with fibrous tissue consistent with electrophysiological and/or impedance properties of the valve annulus.
The fastener is not in intermittent physical contact with tissue (which might indicate location at a moving valve leaflet).
The fastener is not in the vicinity of one or more vulnerable structures with a characteristic electrophysiological signature (such as the AV node).
The fastener is not in the vicinity of one or more selected “tagged” structures, such as a coronary artery 59 marked by a transmitting catheter wire inserted thereto.
The fastener is not in the vicinity of one or more selected mapped structures; such as a bundle of His, or a coronary artery 59 having positions mapped using measurements of electrical fields using electrodes of an electrode catheter inserted thereto.
The fastener is extruded from the catheter (e.g., not shielded by electrically insulating properties of the catheter).
The fastener is oriented as planned for attachment (e.g., attachment by rotation to screw into tissue).
The fastener is tissue-inserted to a planned depth (e.g., has an expected increase in effective impedance due to be being partially embedded in tissue).

It should be noted that some combinations do not require a specific determination of a location's spatial coordinates.

Display of Multimodal Measurements and/or the Compound Model

In some embodiments, the compound model is rendered as a 3-D image (and/or projection of a 3-D image), and the location of features estimated from one or more of the multimodal measurements shown on the 3-D image by markings; for example, differences in the appearance of surfaces represented by the compound model, and/or markers such as symbols or shapes placed alongside surfaces represented by the compound model. Descriptions of different ways of displaying electrophysiological measurements on an image generated from a compound model are described, for example, in relation to block 116 of FIG. 1.

Optionally, an X-ray and/or ultrasound image (static or live-updating) is used as a background onto which positions and movements of other procedure elements (e.g., catheters, probes, and/or the annuloplasty device 112 itself) are projected during the procedure. Projection optionally includes aspects of the anatomical structure; for example, a 3-D image of a valve may be overlaid onto a 2-D image of the heart chambers overall.

Optionally, one or more of the views of FIGS. 4B-4C is shown and updated as the annuloplasty device is implanted to show fastener positions. The positions of the cutaway ends of the view of FIG. 4C (e.g., cutaway surface 406) are optionally changed during implantation to more clearly show activity at the site of a currently implanting fastener. In some embodiments, cutaway surface 406 is positioned adjacent to this site. Optionally, a depth of penetration of the valve annulus by a currently implanting fastener is indicated by showing the fastener's position at or near cutaway surface 406.

In FIG. 2F, the implantation is nearly complete. In the orientation shown, the implantation was begun along the septal wall anterior to the location of the bundle of His 60, and terminates before reaching back around to the bundle of His 60 again.

Partial cinching potentially occurs accidentally during implantation. In some embodiments, this is optionally noted by changes in the imaged environment, and/or a sudden change in measured amounts of regurgitation. In some embodiments, such instances are displayed to the physician, optionally with a warning.

In FIG. 2G, implantation is complete and the annuloplasty device 112 has been cinched, shrinking valve 57, and detached. Optionally, the procedure includes final validation checks, performed before and/or after cinching and detachment.

### Post-Implantation Validation

Returning to FIG. 1: at block 119, in some embodiments, implantation is verified and/or corrected. Some aspects of validation are optionally performed during implantation itself, for example, as already described in connection with the activities of block 120.

Optionally, one or more fasteners 122 is misplaced and/or dislodged during a procedure such that it requires post-implantation removal. In some embodiments, post-implantation imaging is used to identify such instances, and/or to guide a retrieval tool to retrieve fastener 122.

In some embodiments, there is a period of post-implantation adaptation (e.g., of about 30 minutes), during which tension and/or compression induced on the valve annulus by the implant results in initial valve remodeling. In some embodiments, measurements (e.g., by Doppler ultrasound and/or of saline injection retrograde transport) are performed to confirm that results anticipated for regurgitation reduction have actually been obtained. In some embodiments, electrophysiological activity of vulnerable structures is measured, to confirm that no inadvertent electrophysiological block has developed during the period of adaptation (e.g., due to pressure exerted by the annuloplasty device on nervous tissue). In some embodiments, rearrangements of positions of fasteners 122 after valve remodeling are imaged, for example, to confirm that they have not been brought into closer-than-intended proximity to vulnerable structures such as the coronary artery, AV node, and/or bundle of His. Once valve remodeling has stabilized (e.g., changing shown in valve images has slowed and/or stopped), the implantation procedure is optionally deemed complete, equipment is removed from the patient, and the patient is released.

### Mitral Valve Annuloplasty

Reference is now made to FIG. 3A, which schematically illustrates implantation of an annuloplasty device 112 for treatment of regurgitation in a mitral valve 47, according to some embodiments of the present disclosure. Reference is also made to FIG. 3B, which is a schematic flowchart of a method of guiding and monitoring implantation of a mitral heart valve annuloplasty device 112, according to some embodiments of the present disclosure.

Shown in FIG. 3B is an almost fully deployed and attached annuloplasty device 112, comprising sleeve 121, cinch cord 125, fasteners 122, and optional radiopaque markers 123. A control member 124 is shown still connected to fastener 122A, as faster 122A is being attached (e.g., screwed in) to the annulus of valve 47 (this process is described, for example, in relation to FIG. 2E). Also shown is delivery catheter 112A, from which annuloplasty device 112 is being deployed and attached. A multi-electrode catheter 102 is also shown; both devices have been advanced into left atrium 49 of heart 50 from the right atrium 51 via a transseptal access (e.g., across the interatrial septum via the foramen ovale 43).

Also illustrated in FIG. 3B are left atrial appendage 46, and the roots of pulmonary veins 48. Left ventricle 42 is illustrated below mitral valve 47, including papillary muscles 45 of the left ventricle 42, chordae 44, and the aortic root 54.

The blocks 310, 312, 314, 316, 318, 320, 322 of FIG. 3A correspond generally to blocks 110, 102, 114, 116, 118, 120, 119 of FIG. 1, with the substitution of the left atrium 49 for the right atrium 51, of left ventricle 42 for right ventricle 55, and of the mitral valve 47 for the tricuspid valve 57.

Structures to avoid damaging and/or anchoring to directly in mitral valve annuloplasty continue to include the AV node 60A, bundle of His 60, coronary artery 59 (the left branch, in particular), and valve leaflets. Anchoring and/or damage to walls and other non-valvular structures of the left atrium 49 and left ventricle 42 is also avoided.

### Valve Feature Tagging

In some embodiments, valve and peri-valvular features including the valve leaflets, valve annulus (fibrous tissue of the valve annulus, bounded internally by the “hinge” of the valve, and externally by myocardial tissue), and surrounding cardiac tissue are detected, distinguished, and tagged for presentation as an image.

In some embodiments, presentation of features as an image comprises presentation as a 3-D (or 2-D projected 3-D) image, with particular structures distinguishably tagged, for example, by a marker; and/or by differences in color, brightness, saturation, transparency, texture, or another visual characteristic.

Some examples of how different valve and peri-valvular structural elements are distinguished from each other are discussed below. Others, for example, detection of vulnerable structures, are discussed, for example, in relation to FIG. 1.

### Valve Annulus Tagging

Reference is now made to FIG. 7A, which schematically illustrate a method of identifying valve hinge locations, according to some embodiments of the present disclosure.

At block 702, in some embodiments, location voltage measurements 701 (corresponding, for example, to the measurements used to produce the position map of block 110) are taken as an input, and the position map produced. The position map potentially already at least partially identifies the hinge of a valve, based on the location of the “cliff” edge which marks the transition from the approximately valve-aperture transverse surface of the valve annulus to the approximately valve-aperture perpendicular surface of the valve leaflets. Additionally or alternatively, manual or automatic identification of the “cliff” edge is facilitated by restricting the search for a suitable geometrical feature corresponding to a valve hinge to a region along the atrioventricular axis identified as plausible based on electrophysiological measurement of a waveform characteristic of an annular ring position. For purposes of automatic identification, the hinge is optionally identified as comprising a circumferentially extending ring (optionally broken, e.g., at the leaflet boundaries), whereat the elevation angle of surface orientation relative to the plane of the valve annulus is changing in a manner characteristic of the hinge. For example, the change rate is above a threshold, and/or the change rate is fastest at some particular radial distance from the valve annulus.

Block 704 represents intracardiac electrograms, corresponding, for example, to measurements of block 116 of FIG. 1 which map endogenous electrophysiology. It is described herein (e.g., in relation to block 116 of FIG. 1) that electrophysiological waveforms with different characteristics (e.g., relative amplitudes of components) are optionally used to help identify locations such as the AV node and/or bundle of His; and/or to identify locations along the atrioventricular axis. In particular, intracardiac locations having electrophysiological waveforms mid-way between the P-wave dominated (atrial) and QRS-complex dominated (ventricular) are optionally identified as being at the level of the valve annulus.

Block 706, in some embodiments, corresponds to electrical measurements made in spatial coordination with (e.g., at the same places as) the intracardiac electrogram measurements of block 704. Optionally, block 706 includes measurements of local dielectric properties (as indicated in components of electrical impedance affected by nearby tissue, and particularly by contacts with nearby tissue). In particular, the valve annulus ring comprises connective tissue of a composition with impedance properties that are potentially distinct from nearby contractile muscular tissue (e.g., atrial muscle outside the annulus ring), and also potentially distinct (e.g., due to thickness, composition, and/or movement patterns) from impedance measurements made while in contact with the valve leaflets. Distinguishing base on movement patterns is also described in relation to FIGS. 7B and/or 8, herein.

Additionally or alternatively, block 706 includes location voltage measurements (e.g., of externally induced electrical fields), for example of the type optionally used as the location voltage measurements of block 701. Location voltage measurements are voltage measurements indicative of locations; for example, measurements providing a basis for a process of computational reconstruction, e.g., as described in WO 2019/034944.

The same electrode(s) are optionally used for any of the electrical measurements of block 706 (e.g., location voltage measurements and/or dielectric measurements). Simultaneous measurements, in some embodiments, are at least partially separable from each other within a single time series of recorded measurements, for example based on differences in frequency, and/or by using differential analysis techniques in comparison to measurements from other locations. For example, components of measurements due to the externally induced electrical fields can be distinguished based on frequency of the induced electrical fields. Measurement influences due to local dielectric properties are observable at other frequencies, and moreover become particularly pronounced upon making contact with local tissue; to the extent that a sudden large change in impedance is itself potentially indicative of tissue contact using the measuring electrode. The dielectric properties of the relative fibrous valve annulus are different from those of the valve leaflets (on one side) and the cardiac tissue (on the other), leading to a difference in dielectric property-attributable measurement signals upon contact with each of these different tissue types. A “local” component of nearby measurements which are sensitive to both local and diffuse signal sources can in some cases be isolated by signal subtraction (e.g., when one of the measurements is in a relatively quiet local environment), or by another differential analysis technique.

In some embodiments, positions and/or characteristics of the location measurements of block 706 are used to assign positions to the intracardiac electrogram measurements of block 704 within the position map of block 702.

At block 708, positions of locations tagged as “hinge” (valve annulus) positions are output, based on processing of inputs to block 709 from blocks 702, 704, and 706. In some embodiments, measurements made from positions which are generally at the level of the valve annulus are validated by intracardiac electrogram measurements which sufficiently correspond to measurements expected from the level of the valve annulus along an atrioventricular axis (e.g., as described in relation to block 116 of FIG. 1). Local dielectric properties due to tissue characteristics (thickness, tissue type, and/or movement) provide a further distinguishing characteristic (e.g., distinguishing valve annulus from valve leaflets and/or myocardial tissue). From detailed structural (shape) measurements of the heart lumen, in some embodiments, there may be distinguished the position of the edge of a “cliff” which drops suddenly into the ventricle from the atrium, and this also is a marker of the valve annulus position. The information from any one or more of these types of measurements optionally is used (including used jointly) to create a tissue-type tagged model of the valve annulus.

In some embodiments, structures of the valve are in part differentiated using analysis of the temporal frequencies of motion of different structural elements of the valve, for example as described in relation to FIG. 7B.

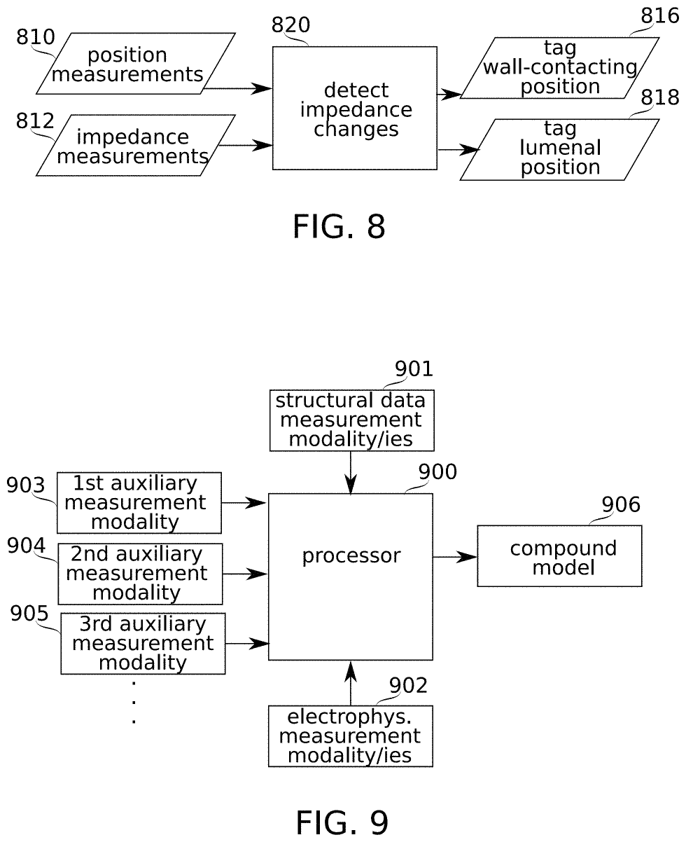

Optionally, locations of valve hinge vs. valve leaflets are distinguished at least partially on the basis of impedance measurements indicating wall contact (for example, as described in relation to FIG. 8); for example, contacts of a relatively stationary probe with valve leaflets may tend to be intermittent, compared to contacts with the valve hinge (annulus).

### Valve Leaflet Tagging

Reference is now made to FIG. 7B, which schematically illustrates a method of using time-frequency decomposition to distinguish components of heart structure as belonging to different structures, according to some embodiments of the present disclosure. Reference is also made to FIG. 5, which schematically represents time traces of respiration (trace 501), and body surface ECG (trace 502).

As imaging measurements (e.g., based on electrical impedance tomography, or another imaging method) are made of the region of a heart valve over the course of several heart and respiratory cycles, different parts of the valve move in different ways. For example, the valve annulus (e.g., of the tricuspid valve) experiences longitudinal motion in response to contractile actions of surrounding cardiac tissue, and may experience slower motions as a result of lung and diaphragm movements. Valve leaflets may also have movements at higher frequencies (e.g., harmonics of the heartbeat frequency): for example a brief partial opening at a phase other than the valve's main opening. Where imaging data is obtained, e.g., using internally placed electrodes positioned at a vantage point substantially along an axis of motions of the valve (e.g., an atrioventricular axis, in the case of the tricuspid valve or the mitral valve), the valve may be considered as laid out approximately across an X-Y axis-defined plane, and its motions may be understood as happening approximately toward and away from the vantage point of the images along a Z axis (e.g., atrioventricular axis). This motion can be understood as “painting” different parts of the X-Y axis with different cyclic motion-pattern “colors”, carrying information that distinguishes, e.g., valve leaflets from the valve annulus itself.

In some embodiments, different cyclic movements of tissues belonging to different structural parts of the valve are differentially labeled by what is known in the field of image feature detection as “blob detection”. Areas “colored” with high power in harmonics of the heartbeat frequency are labeled as valve leaflets; other areas are not. In some embodiments (for example as shown in FIG. 7B), spectral power (amplitude variation of the measured waveform over time) is decomposed to (attributed to) respiratory frequency, cardiac cycle frequency, higher cardiac cycle frequency harmonics, and residual processes. The resulting distribution of spectral power is categorized, e.g., using a Hessian operator-based method of blob detection, or another method, for example, a machine learning-based method that uses established associations of feature locations to measurements at those locations as input, and from this input learns which measurements indicate which features.

In some embodiments, image feature detection comprises receiving measurements 710, 712, 714 of the breathing cycle (e.g., trace 501 of FIG. 5), body surface ECG (cardiac cycle, trace 502), and higher harmonics of the body surface ECG (respectively) to a decomposition module 716, which uses measurements 710, 712, 714 to decompose and tag the time- and space-indexed imaging measurements of block 716; tagging the spatial locations according to the magnitude of their various cyclic motions—breathing 710A, cardiac cycle 712A, higher cardiac cycle harmonics 714A, and residuals 718.

Valve leaflets may, additionally or alternatively, be modeled parametrically using constraints established by impedance measurements obtained over time. For example, each valve leaflet is modeled as tissue anchored on one side to the valve annulus (with a certain parametrically defined circumference), over a certain parametrically defined distance of the circumference, and with certain parametrically-defined shapes of its commissural sides. The effects of any particular set of parametrically defined leaflets on impedance measurements can be determined by using impedance properties of the tissue, and well-known equations of how impedance changes modify electrical field distribution. The impedance measurements, accordingly, constrain the parametrically defined configuration of valve leaflets that is actually present. This configuration can be found, for example, by an iterative process of valve parameter adjustment that leads to a closer fit between actual measurements (and in particular, in some embodiments, the component of actual measurement attributable to valve motions) and predicted measurements.

### Tissue Wall Contacts

Reference is now made to FIG. 8, which schematically represents detection of wall contacts, according to some embodiments of the present disclosure.

In some embodiments, locations in contact with tissue surfaces are distinguished using impedance measurements. Block 810 represents position measurements made (e.g., using impedance measurements) by a probe (e.g., an electrode probe) moving around within the heart chambers, and block 812 represents measurements of impedance sensed by an electrode of the probe between itself and an external electrode. As the electrode approaches and then contacts surface of the heart chambers, impedance rises. Impedance rises corresponding to the characteristics of lumenal wall surface contacts are detected at block 820, the output of which is used to tag positions as wall contacting (block 816) or lumenal (non-wall contacting) (block 818).

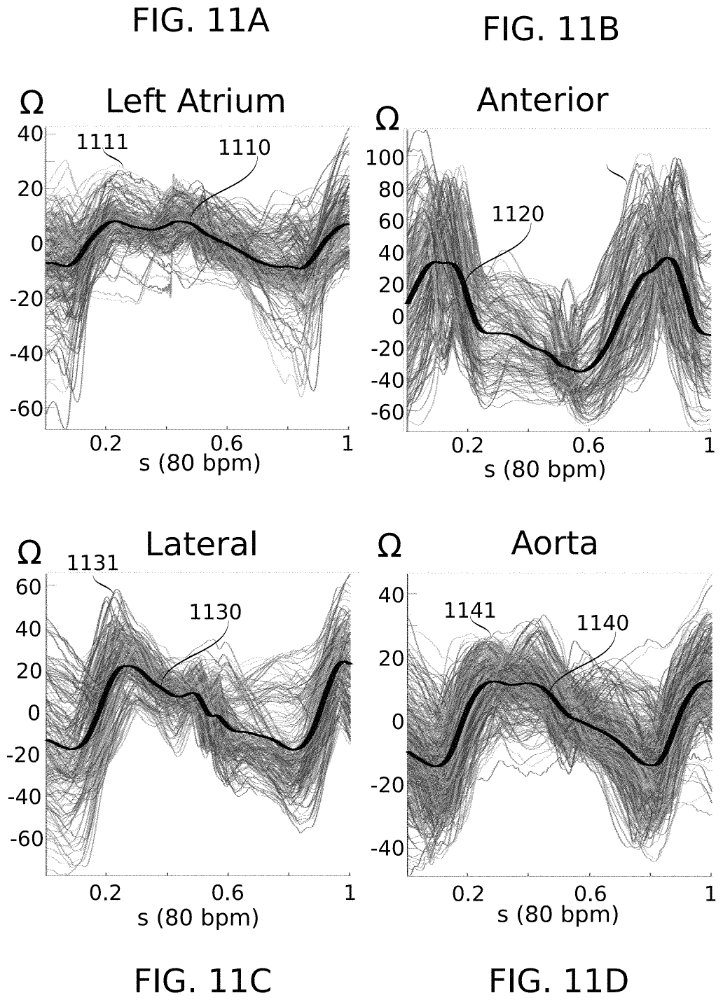

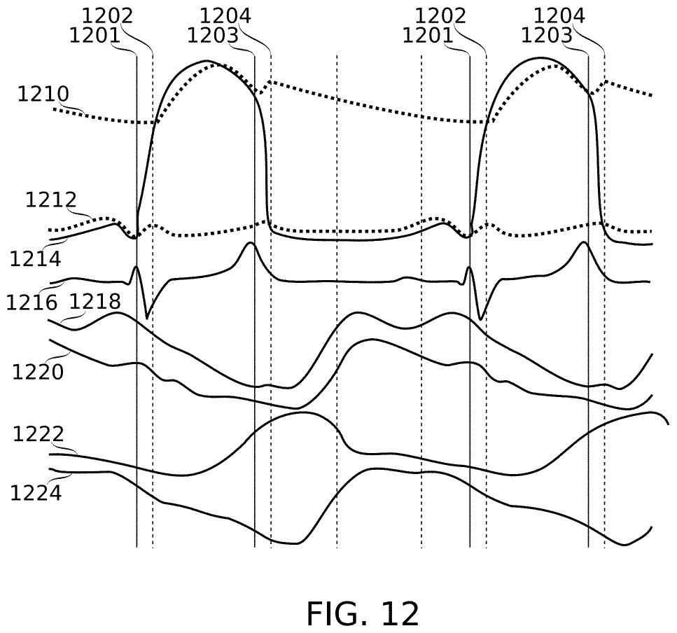

In some embodiments, tissue wall contacts in the peri-valvular region which are not known (e.g., by other segmentation tests such as those of FIGS. 7A-7B) to be of another structural type such as the valve annulus or valve leaflet are assigned as cardiac muscle tissue. Characterization using identification of which cardiac muscle tissue surface area (which “wall” area) is being contacted is described further in relation, for example, to FIGS. 11A-12, herein.

### Systems for Combined Modality Imaging

Reference is now made to FIG. 9, which is schematic diagram of a system for monitoring and/or guiding annuloplasty device implantation, according to some embodiments of the present disclosure.

Computer processor 900 is configured to execute computer code instructions for integrating data acquired from data measuring and/or processing subsystems supporting a plurality of measurement modalities (for example, measurement modalities supported by data measuring and/or processing subsystems 901, 902, 903, 904, and/or 905) into a compound model 906 of a heart. In some embodiments, compound model 906 models at least a heart valve and its vicinity. Integration of data from subsystems 901-905 comprises, for example, use of simultaneity of measurements obtained using different subsystems 901-905, and/or another method of establishing coordination between different measurement modalities, for example as discussed in relation to FIG. 1. While any of the measuring and/or processing subsystems 901-905 is optional, there are in general at least two of them in operation to provide inputs to processor 900. In some embodiments, the system of FIG. 9 includes a display, through which the processor displays output.

Compound models 906 and aspects of their determination are discussed in relation to several different embodiments herein; for example, in relation to FIGS. 1, 3A, and/or 7A-8. Other example descriptions follow, each an example of a relevant plurality of subsystems indicated:
Systems configured to map a vascular lumen extending alongside a body cavity. In some embodiments, first and second probes moving within each respective lumen comprise separate modalities of position measurement (two instances of subsystem 901). The compound model 906 optionally comprises, for example, a combined spatial representation of the two lumens, and/or a model of distances between first lumen locations and second lumen locations. As an example, the first lumen may be a position within an atrioventricular space, and the second lumen may be a coronary artery. Optionally, first lumen locations and distances to the second lumen which are “too small” (e.g., below a threshold) are considered “dangerous” for certain operations such as implantation/attachment (e.g., posing a risk of perforating the coronary artery), and indications are produced to help an operator avoid performing those operations in the dangerous areas.
Systems configured to locate a heart valve annulus along an atrioventricular axis (or another axis along which electrophysiological measurements show characteristic variation). In some embodiments, electrophysiological signals are measured from probe positions extending between an atrial side and a ventricular side of a heart valve annulus (subsystem 902), while probe positions are also measured (subsystem 901). Position-dependent characteristics of the electrophysiological signals are used to identify which positions are apparently within the region of the heart valve annulus. Operations performed using such systems are also described, for example, in relation to FIGS. 18, herein.
Systems configured to detect valve leaflets. In some embodiments, measurements of electrical signals indicative of impedance from one or more electrodes located near a cardiac valve (subsystem 903, comprising sensor and processing to perform time-series intracardiac impedance measurements) are processed to determine the presence and/or details of electrical signals characteristic of proximity to a leaflet of the cardiac valve. Position information (e.g., obtained using a structural data measurement modality 901) is optionally used to structure a spatial model of the position measurements; for example to associate impedance readings to particular locations and allow mapping of valve leaflet positions and/or movements.
   In some embodiments, position is “gated”, so that only measurements made at certain preferred positions (e.g., within a preferred region) are used in the mapping. For example, it is may be identified that certain measurements are preferably made at a certain region, e.g., because it is in an appropriate general proximity to expected positions of a structure which is to be characterized. It may be difficult to deliberately approach this region with a measuring electrode and steadily remain within it, due, e.g., to ongoing motions of the heart. Position-gating measurements assists by selecting those measurements which occur when the measuring electrode happens to be within the preferred region, and rejecting those occurring outside of it.
   In some embodiments, the preferred positions are themselves defined using inputs from one or more electrophysiological measurement modalities 902, e.g., using location along an atrioventricular axis determined using electrophysiological measurements. Operations performed using such systems are also described, for example, in relation to FIGS. 7B and/or 13-19, herein. In some embodiments, timing of impedance signal events is related to the known pattern of motions of the heart valves using a synchronizing indication, for example, an ECG recording (subsystem 902). Optionally, another synchronizing indication is used; for example, one of subsystems 904-905 may comprise a heart noise sound recorder, a pressure sensor, a blood volume sensor, a Doppler signal sensor, or another sensor producing a signal at times that can be directly associated to characteristic movements of the heart during the heartbeat cycle.
Systems configured to identify hinge boundaries between a heart valve annulus and heart valve leaflets. In some embodiments, inputs are received which measure a time course of an electrical signal indicative of impedance due to motion of tissue; more particularly, in some embodiments, at a plurality of probe positions in proximity to one or both of the heart valve annulus and the heart valve leaflets. This corresponds to an example of subsystem 903, comprising an electrode sensor and processing to perform time-series intracardiac impedance measurements. Using position information of the probe (e.g., obtained using a structural data measurement modality 901), relative spatial locations of the probe positions within the heart are determined to be valve hinge positions, based on being at locations between valve annulus locations (having one type of motion signal recorded using subsystem 903) and valve leaflet locations (having another type of motion signal recorded using subsystem 903). Operations performed using such systems are also described, for example, in relation to FIG. 7A, herein.
Systems configured to locate a structure of the electrical conduction system of the heart. In some embodiments, measurements of intracardiac electrophysiological signal waveforms made are at intracardial probe positions (subsystem 902), and spatial locations of the probe determined separately (subsystem 901). The electrophysiological waveforms and spatial information are processed together to identify at least one of the spatial locations as being at the position of the electrical conduction system structure. Operations performed using such systems are also described, for example, in relation to block 101 of FIG. 1, herein.
Systems configured to identify heart wall contacts. In some embodiments, a procedure entails identifying and/or confirming the identification of which portion of a heart lumenal wall is being contacted by electrodes within the heart; for example to confirm that a transseptal crossing is about to be made in the interatrial septum itself, instead of into the adjacent aorta. Subsystem 903 may be configured to measure impedance from those electrodes, producing time-series impedance data with signals characteristic of the wall location that the electrodes contact. Interpretation of these signals, in some embodiments, makes use of electrophysiological measurements (obtained using subsystem 902) which establish the relative timing of ECG events and impedance signal features. Operations performed using such systems are also described, for example, in relation to FIGS. 8 and/or 11A-12, herein.

In some embodiments, systems are configured to use a compound model (e.g., as may be generated by any of the above-described systems) to generate a specification of implantation positions within a heart posing a risk; for example, risk of damage to a right coronary artery, a bundle of His, or another functionally crucial region of the heart. Positioning of a portion of an implantable device (e.g., a fastener of an annuloplasty device) may be tracked according to one or more of subsystems 901, 902, 903, and compared with the compound model to reach targeted structures, and/or to help reduce a risk of causing damage to identified structures.

In some embodiments, systems are configured more particularly to monitor the implantation of a fastener for an annuloplasty device into a valve annulus. In some embodiments, measurements of an electrical signal indicative of impedance using the fastener as an electrode are received while the fastener is being brought to an implantation position (instance of a subsystem 903). Again, in some embodiments, locations of other structures have been previously mapped, for example according to operations outlined with respect to any of the above-described systems.

In some embodiments, a system according to FIG. 9, or another system comprising processor and suitably memory-stored instruction, is configured to implement one or more feature-finding algorithms, for example:
Algorithms to automatically locate a hinge of a heart valve annulus.
Algorithms to define a nominally safe pathway extending along and between the locations of two circumferentially extending portions of the heart comprising a hinge portion of the heart valve, and a portion of a coronary artery.

In some embodiments, subsystem 901 comprises devices and/or software implementing a measurement modality which provides data supporting production of a 3-D structural image of the modeled portion of the heart. Subsystem 901 itself may comprise, for example, computer code configured to receive electrical field measurements (e.g., from a plurality of electrodes of a probe inserted into the heart) and convert them into positions associated with the measurements. Optionally, subsystem 901 also comprises electrical field measurement hardware such as an electrode probe (e.g., having a plurality of electrodes at a known relative distance), a measurement controller functionally configured to measure voltages, currents, and/or impedances from electrodes of the electrode probe, a catheter over which the electrode probe is delivered to a lumen of the heart, analog-to-digital conversion circuitry, a memory store for recorded measurements, and/or a digital communication link for transmitting measurements made by subsystem 901 to computer processor 900.

Additionally or alternatively, in some embodiments, subsystem 901 comprises 3-D ultrasound equipment, configured to measure, record, and transmit 3-D ultrasound measurements and/or images to computer processor 900.

In some embodiments, subsystem 902 comprises devices and/or software implementing a measurement modality which measures electrophysiological signals produced by endogenous and/or stimulus-evoked activity of cardiac tissue. In some embodiments, subsystem 902 includes, for example, one or more electrodes, a probe carrying the one or more electrodes, a measurement device for voltage, current and/or impedance, analog-to-digital conversion circuitry, a memory store for recorded signals, and/or a digital communication link for transmitting measurements made by subsystem 902 to computer processor 900.

In some embodiments, subsystems 903-905 (there may be one, two, three, or more such subsystems; three are shown for purposes of description) comprise devices and/or software (“equipment”) implementing other measurement modalities, and in functional communication with computer processor 900. Examples include:
Body surface ECG equipment.
Respiratory cycle motion measurement.
X-ray imaging equipment.
Echocardiography equipment; configured for example to measure valve shapes and/or Doppler signals due to regurgitation.
Equipment to inject saline and measure retrograde saline transport (e.g., as a measurement of regurgitation).
Catheter and recording and/or generating equipment configured to insert a wire electrode to a blood vessel (for example, a coronary artery), and record and/or generate an electrical signal therefrom.
Catheter and recording and/or generating equipment configured to insert an electrode catheter (e.g., a catheter comprising a distal probe with a plurality of electrodes along its length) to a blood vessel (for example, a coronary artery), and record electrical signals therefrom which are indicative, under a suitable voltage-to-spatial transformation, of the spatial positioning of the blood vessel.

### Coronary Artery Proximity and Penetration Detection

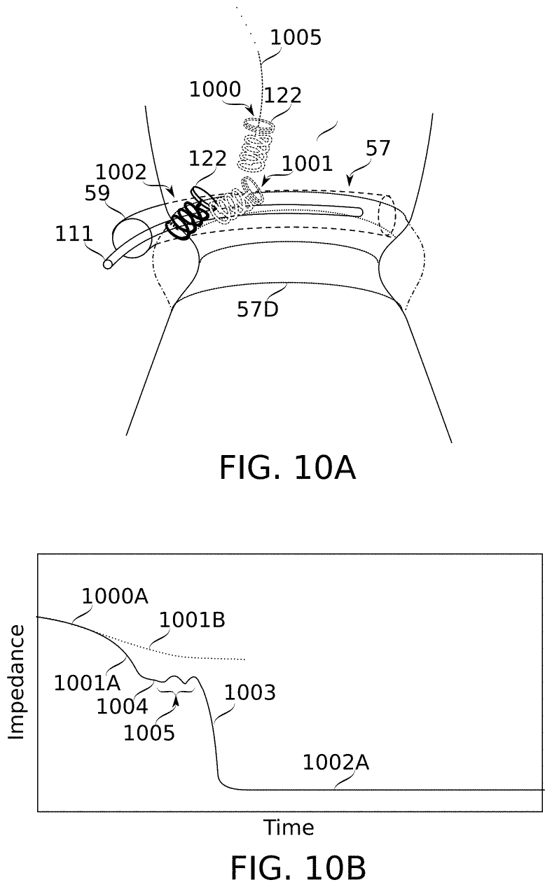

Reference is now made to FIG. 10A, which schematically represents coronary artery proximity and penetration by a device fastener 122, according to some embodiments of the present disclosure. Reference is also made to Figure JOB, which schematically represents features of coupling measurements potentially useful to detect changes of coronary artery proximity and penetration by a fastener, according to some embodiments of the present disclosure. Figure JOB illustrates idealized measurements of impedance over time, with, e.g., measurement noise suppressed to assist in illustrating main features.

In some embodiments of the present disclosure, a coronary artery (e.g., in the right atrium, the right coronary artery) is at potential risk for damage during a valve annuloplasty, or optionally another structural procedure performed in heart lumen regions underlying the coronary artery. FIGS. 10A-10B illustrate methods of avoiding and/or detecting coronary artery damage due to contact and/or penetration by a fastener 122. Fastener 122 may be, for example, a fastener of a valve annuloplasty device, or another implantable device.

FIG. 10A shows fastener 122 in three positions 1000, 1001, and 1002 corresponding to times and relative impedances 1000A, 1001A, and 1002A of Figure JOB, respectively. Fastener 122 is connected to an electrical sensing and/or driving system, for example via wire 1005, which for clarity of illustration is shown only in a distal portion thereof for the case of position 1000. Also for clarity of illustration, the device being fastened with fastener 122 is omitted from the drawing. Its relationship with fastener 122 may be, for example, as illustrated and/or discussed in relation to FIG. 3B, or another figure herein.

At position 1000, fastener 122 approaches the vicinity of catheter wire 111. Catheter wire 111 has been previously inserted into coronary artery 59; for example as described in relation to FIG. 2B. In some embodiments, for example, catheter wire 111 is a catheter probe, having on it electrodes. At least one of catheter wire 111 and fastener 122 is driven to generate a small (e.g., <1 mA) electrical current (e.g., an AC electrical current at about 10-40 kHz); similarly, at least one of the two is configured for use in sensing one or more parameters of electrical coupling between the two (e.g., impedance). The trace in the region of time/impedance point 1000A represents this coupling in the form of impedance.

At position 1001, fastener 122 approaches coronary artery 59 more closely, and the coupling increases. This may be measured, for example as a drop in impedance to the level of the region of time/impedance point 1001A. There may also be impedance change effects as a function of orientation, depending on the geometry and electrical conductivity characteristics of fastener 122.

This allows using impedance between fastener 122 and catheter wire 111 in a method of judging relative distance between the fastener and the coronary artery.

One way that relative values can be used in judgement is by noticing the difference between impedances achieved upon approaches two different areas of valve annulus 57D. Approach to a “landing point” (fastening position) further from catheter wire 111 (and the coronary artery it occupies) will potentially not increase coupling as much and/or as quickly as an approach to a landing point which is closer, and correspondingly at greater risk for introducing a complication of coronary artery penetration. Region 1001B (Figure JOB) shows such a reduced change in coupling as a reduced change in impedance. Thus, different candidate fastening positions can be compared, and an apparently less-risk position selected.

A plateau region (plateau 1004, for example) may indicate that contact with tissue resistant to further movement has been made, particularly if the plateau persists with minimal reduction in response to attempts to advance fastener 122 further.

Region 1005 indicates semi-cyclical changes in impedance which may, in some embodiments, appear as a helical fastener 122 is driven into tissue (e.g., as a tip of fastener 122 rotates toward and away from catheter wire 111. Other fastener geometries may be associated with different characteristic “penetration” profiles of a coupling measurement such as impedance.

Through region 1003, a penetration event is occurring, representing a rapid increase in coupling (drop in impedance) as a tip of fastener 122 intrudes into coronary artery 59. The impedance drop may reflect the lower resistivity of blood in comparison to the solid tissue of the valve annulus. At region 1002A, a new, lower plateau of impedance is reached.

This provides a potentially immediate indication to an operator such as an implanting physician that a complication involving the coronary artery has likely occurred. This allows quickly halting the advance of a fastener before more damage occurs. There can also be taken rapid remedial steps to repair the damage which has occurred, e.g., by cauterization, or switching to another mitigation procedure.

It is provided, in some embodiments, that an alarm (visual, haptic, and/or auditory; optionally, an intrusive alarm—loud and/or garish, for example) is used to warn an operator of the likelihood of a penetration event, detected by a sudden impedance drop. The physician optionally sets the threshold for the alarm in terms of coupling change (e.g., impedance drop) magnitude and speed. Optionally, one or more presets may be provided.

Impedance changes can occur for different regions during a procedure, and it is a potential advantage to reduce the production of false positive alarms.

Optionally, in some embodiments, production of an alarm upon detection of one or more of the coupling/impedance waveform characteristics just described is gated by at least one additional criterion that helps increase the likelihood that the event detected is really a penetration event. In some embodiments, the additional criterion is provided by measuring the position of fastener 122 along an atrial-ventricular axis, for example as described in relation to FIGS. 2E and/or 7A. Accordingly, only when the position is determined to be within the region of the valve annulus will a determination of a likely penetration event be made and/or converted to an alarm to the operators.

It should be noted that coupling between catheter wire 111 and fastener 122 may be measured in another way, for example using vibrations (e.g., as described in relation to FIG. 2E), or another electromagnetic measurement method. The monitoring of impedance as a measure of coupling (described as an example here) has been found to be notably sensitive to penetration events.

It is noted that relative position (e.g., distance and/or direction) between a first probe and catheter wire 111 within a coronary artery may be measured while tracking positions of the first probe by a separate indication—for example, measurements of voltages within a plurality of crossing electrical fields. This can be used to generate a map of regions close enough to the coronary artery to raise potential concern; for example, concern for risk of damage during device implantation. In some embodiments, proximity to the coronary artery for another probe (e.g., the fastener 122 itself), can be tracked using the same separate indication of position. Proximity to the coronary artery can then be determined from the map. It is noted that the pre-mapped proximity determinations can be used without direct coupling (e.g., current and/or voltage measurement) between the fastener 122 and catheter wire 111. Furthermore, once mapping is performed, and catheter wire 111 can be removed from coronary artery 59.

### Valve Mapping Using a Synchronization Signal

### Synchronization Used to Interpret Motion Indications in Impedance Signals

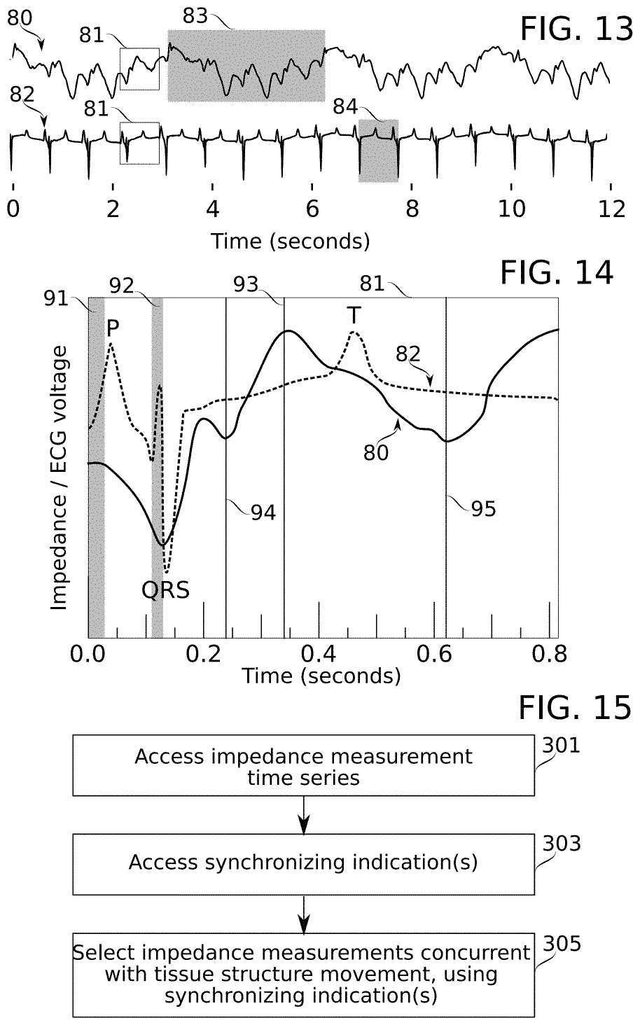

Reference is now made to FIGS. 13-14, which illustrate relative timings of body surface ECG events and intracardial impedance measurements indicative of movements of the right atrioventricular valve (tricuspid valve), according to some embodiments of the present disclosure.

Time series 82 represents a body surface ECG time course, displayed over the course of several seconds comprising several heartbeats. Time series 80 is an impedance-difference time series, as further explained below.

Two simultaneous impedance time courses were measured, each using a different intracardial electrode referenced to a body surface electrode. The intracardial electrodes were positioned each offset from the other and “above” (that is, in the atrial lumen) the leaflets of a right atrioventricular valve (RAV) in a living pig heart. One electrode was furthermore positioned relatively further from the RAV, where it recorded a relatively weaker signal due to movements of the RAV leaflets.

For purposes of description, the RAV is said herein to lie approximately in an X-Y coordinate plane, while positions above or below that plane have some corresponding Z-coordinate distance from the plane of the RAV. Thus, the two intracardiac electrodes may be said to have different Z-axis coordinates, and a Z-axis distance between them.

Time series 80 is an impedance-difference time series obtained as the difference between these two traces, subtracting the weak RAV signal time series (measured further from the valve) from the stronger (the order of subtraction maintains the normal polarity of the impedance-measured motion signal). The subtraction procedure is optional, and carries the potential advantage of cancelling a portion of other “shared” signals due to heart wall motion (e.g., repeating with about the period of the length of shaded box 84) and/or respiration (repeating with about the period of the length of shaded box 83). It may be seen that the respiration signal in particular is not completely canceled, but the difference operation still serves to potentially improve the starting signal-to-noise ratio. Optionally, the respiration signal is removed from time series 80, for example, by filtering out frequencies typical of respiration, by reconstructing and subtracting a “typical” respiration movement cycle, or by another signal processing method.

The time series data 80, 82 of box 81 of FIG. 13 are shown magnified (and superimposed) in FIG. 14. Within box 81 in FIG. 14, the P and QRS waveforms of ECG time series 82 are marked. The P waveform corresponds to arterial contraction, the QRS waveform corresponds to the onset of ventricular contraction, and the T wave corresponds to ventricular repolarization.

Waypoints indicated for impedance time series 80 relate to movements of the leaflets of the RAV. Shaded box 91 indicates an about 30 msec delay between the onset of the P wave (and atrial contraction) on the left; and, on the right, the onset of a downward trend in impedance time series 80. This downward trend is indicative of movement of the RAV leaflets away from the intracardial electrodes making measurements; or in this case, opening of the RAV during atrial contraction to allow the passage of blood. An interval of about 20-30 milliseconds between P wave onset and impedance signal downward deflection is optionally considered within a typical predicted range.

Without commitment to a particular theory of operation, the reduction in impedance may be understood as a result of moving a relatively insulating membranous structure (the valve leaflets have a higher impedance than the surrounding blood) to a more remote position from the measuring intracardiac electrode, making it correspondingly less effective in blocking current flow to the measuring electrode.

Other things (e.g., X-Y coordinate positioning) being equal, the magnitude of the effect for a given motion amplitude is generally larger for measurements from a relatively nearer electrode, for example, according to an inverse power of the distance. In some embodiments, the Z-axis distance between the two measuring electrodes is known, and used as a calibrating indication. The impedance time series provides an approximate measure of the falloff in valve leaflet motion signal over the known Z-axis distance, and the signal falloff/distance in turn may be interpreted as characteristic of a certain absolute distance, for example as later described herein.

Box 92 shows another timing relationship—the onset of the QRS wave in time series 82 (left side), and the subsequent onset (right side) of a returning increase in impedance time series 80. An interval of about 20-30 milliseconds between QRS wave onset and impedance signal upward deflection is optionally considered within a typical predicted range; an interval of about 20 milliseconds is shown).

The increase corresponds to leaflets of the RAV moving back toward the measuring electrodes, in a ventricular-to-atrial direction. The increase in impedance terminates at mark 93 with the closure of the RAV, due to contraction of the right ventricle. Of note is a minor but characteristic impedance fluctuation resulting in a transient local minimum at mark 94. This is related to the operation of other heart valves (pulmonary and/or aortic valves) introducing a transient pressure fluctuation that momentarily reverses leaflet movement. Closure may occur, for example, about 100-200 milliseconds after the end of the QRS complex.

Following valve closure at mark 93, the impedance time series again decreases, corresponding to the valve leaflets again moving away from the measurement electrodes. This motion is a consequence of lowered ventricular pressure as the right ventricle expands again. After the T-wave and from mark 95 (another local minimum of the impedance time series 80), the valve returns to its (substantially closed) resting position, in preparation for another heartbeat cycle.

It may be noted that the above-described features repeat reliably along the displayed interval of impedance time series 80. Filtering out the respiratory movement signal may potentially emphasize these features still further.

However, it should be understood that the trace was obtained with the measurement electrodes in a stable position, whereat the received leaflet motion signal was strongly received. In the case of a moving electrode, the leaflet motion signal amplitude is found to rise and fall irregularly, and may, moreover, be confounded by other impedance signals (e.g., caused by other heart motions) in different degrees. Individual components of the leaflet motion signal themselves may not occur in constant proportion as a function of X-Y position; for example, since different parts of the leaflet move to different degrees along the Z-axis. Furthermore, heartbeat timing itself is somewhat irregular from beat to beat. These are examples of issues which make leaflet motion signal analysis based solely on features of the impedance time series potentially difficult and/or unreliable.

Accordingly, the use of a synchronizing signal provides potential advantages for analysis of the leaflet impedance measurement time series, by allowing the definition of an analysis time window having a reliable relationship to expected time(s) of leaflet movement.

The P and QRS waveforms of a body surface ECG offer particular potential advantages for use as synchronizing indications. The P wave is both sharply defined in time, and close in time (e.g., relative to the heartbeat cycle overall) to the onset of valve opening. The QRS wave is also sharply defined, and close in time to the onset of valve closing. Moreover, changes in valve position occur rapidly during short episodes following these synchronizing indications. Thus, not only can analysis windows potentially be defined reliably relative to actual motions (e.g., within about 10 msec), they also can be kept short, which helps to minimize contributions from potentially confounding signals.

A number of other synchronizing indications of heart valve motions are available, which may be used alternatively or additionally. Heart sounds correspond to the events of heart valve closures, for example. Taken alone, however, heart sounds may be somewhat less satisfactory as a synchronizing indication—at least for the RAV or mitral valves—compared to the ECG waveform events just described. For example, RAV closure happens at about the time of mark 93, but this is a time with a potentially lower rate of impedance change before and/or after. There may also be confounding effects from the nearly simultaneous closure of the mitral valve. The sound of the pulmonary and/or aortic valves closing is more indirectly related to RAV/mitral valve opening (and thus potentially a more variable synchronizing indication).

Synchronizing indications are additionally or alternatively provided, for example, using ultrasound (e.g., following the course of a Doppler motion signal of blood using transthoracic ultrasound), or intracardially measured ECG signals. Optionally, an impedance time series itself is processed to form a synchronizing indication—for example, impedance measured from a fixed electrode, and/or an impedance measurement time series showing the heart wall motion signal more clearly (e.g., a raw intra-atrially recorded impedance measurement time series), allowing heart wall motions to become a synchronizing indication for heart valve motions.

While the example of FIGS. 13-14 is described in relation to the impedance-based measurement of motions of the RAV, it should be understood that the same basic procedure applies, changed as necessary, to the identification of motion signals from other heart valves (e.g., the mitral valve, pulmonary valve and/or aortic valve), and/or to the identification of motion signals from other structures, potentially including papillary muscles and/or chordae. One change to be implemented with such cases of alternative structures is to reposition intracardiac measurement electrode(s) to positions where the alternative structure's motion signal can be discerned. This may include positioning electrodes nearer to the alternative structure than to other structures (particularly those which might be moving at the same time), and optionally positioning electrodes with a particular directional order—e.g., one closer to the alternative structure than another.

Impedance Signal Synchronization with Synchronizing Indication(s)

Reference is made to FIG. 15, which is a flowchart schematically outlining a method of selecting relevant impedance measurements from an impedance measurement time series, according to some embodiments of the present disclosure. Embodiments of FIG. 15 should be understood as being implemented on computerized processing hardware.

At block 301, in some embodiments, at least one time series of impedance measurements is accessed. The impedance measurements are made using one or more electrodes positioned within a heart; optionally the one or more electrodes are all carried on a single probe, for example, a catheter-delivered probe. In some embodiments, impedance measurements are made between an individual intracardial electrode and a reference electrode outside the chambers of the heart (e.g., a body surface electrode). In some embodiments, impedances are self-impedances (of individual electrodes in their present electrical environment). In some embodiments, impedance measurements are made between a plurality of intracardial electrodes positioned within a heart chamber. In some embodiments, measurement between a plurality of intracardial electrodes comprises injecting current to a first electrode of the intracardial probe at a first frequency and measuring voltage between said first electrode and a second electrode of the intracardial probe at said first frequency.

If more than one time series is accessed, the plurality of time series of impedance measurements comprise concurrent time series. Optionally, the time series of impedance measurements undergoes pre-processing for enhancement of the motion signal of a particular tissue structure; for example, a subtraction and/or frequency filtering pre-processing stage. In some embodiments, the particular tissue structure comprises leaflets of a cardiac valve, and the characteristic movements comprise movements of the leaflets during the cardiac cycle as the cardiac valve opens and closes.

At block 303, in some embodiments, one or more synchronizing indications is accessed. A synchronizing indication comprises measurements of an event (and in particular, the timing of the event) which itself occurs with a stereotypical temporal relationship relative to one or more characteristic movements of the particular tissue structure. The measured events may comprise, for example, waveforms of an ECG (e.g., P waveform, QRS waveform, T waveform, or another ECG feature), body sounds (e.g., sounds of heart valve closings), ultrasound-recorded events (movements in an ultrasound image, and/or Doppler signals in an ultrasound image), and/or another event.

In some embodiments, the “stereotypical temporal relationship” comprises simultaneity, an offset in time, and/or a range of offsets in time between the event of the synchronizing indication and the characteristic movement. The event of the synchronizing indication may occur before, after, and/or during the characteristic movement, insofar as both the event and the characterizing movement are optionally associated with non-instantaneous durations. For an event and/or characterizing movement which is defined as instantaneous (e.g., onset, peak, valley, or other moment of an event or characterizing movement), either may be first, or they may be simultaneous. Preferably, the minimum offset in time is less than half of the duration of a heartbeat cycle, e.g., less than 400 msec. In some embodiments, the offset it time is less than 200 msec, less than 50 msec, less than 30 msec, or less than 20 msec. Shorter offset times confer a potential advantage in the precision of “window” selection, insofar as this reduces the extent to which beat-to-beat heartbeat variability may affect timing results. Optionally, the stereotypical temporal relationship is qualitative rather than expressed in terms of a time duration. For example, the characterizing movement may comprise the next opening or closing of a valve, as determined from the at least one time series of impedance measurements, assuming that the opening or closing is associated with an identifiable signal feature such as a local minimum or maximum.

At block 305, in some embodiments, impedance measurements from the at least one time series are selected, using the synchronizing indication. The selected impedance measurements are those estimated to be concurrent (e.g., concurrent for a particular heartbeat cycle) with the characteristic movement of the particular tissue structure.

In some embodiments, the selected impedance measurements may be used to identify and/or characterize a position and/or movement of, and/or image, an intracardial structure (e.g., leaflet(s) of a cardiac valve, or a wall of a heart chamber). In some embodiments, a signal within the selected impedance measurements may be used to identify and or characterize a position and/or movement of, and/or image, an intracardial structure.

### Applications of Motion-Synchronized Impedance Measurement Time Series Data

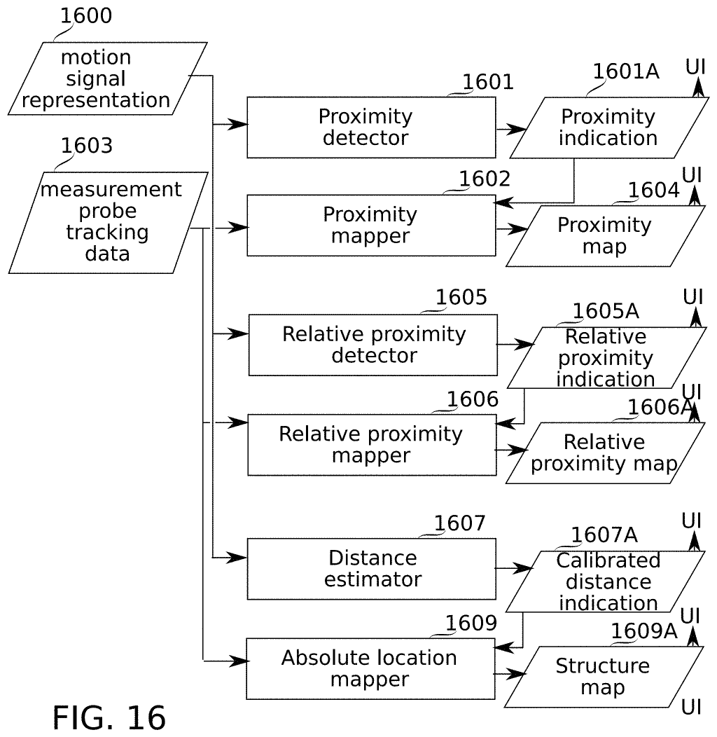

Reference is made to FIG. 16, which is a diagram schematically outlining various embodiment implementations which convert impedance measurements from an impedance measurement time series into estimates characterizing the position of a particular tissue structure, according to some embodiments of the present disclosure. In some embodiments, the impedance measurements are selected according to the method of FIG. 15. In some embodiments, the impedance measurements are measured, for example as described in relation to FIGS. 13-14.

There are several types and applications of “position characterizing” which may be performed, according to various embodiments of the present disclosure. FIG. 16 outlines a number of these according to their features—processing steps, inputs, and/or outputs. There is no particular requirement for any particular embodiment to have all of these modules; they should each be understood as being optionally embodied in any combination and/or in the alternative. Instances where a first processing block (e.g., blocks 1602, 1606, 1609) uses an input generated by a second processing block are optionally implemented as an integrated processing module.

Embodiments of FIG. 16 should be understood as being implemented on computerized processing hardware. In some embodiments, a system implementing the modules and/or methods of FIG. 16 additionally comprises the modules and/or methods described in relation to FIG. 16. Additionally or alternatively, in some embodiments, a system implementing the modules and/or methods of FIG. 16 includes measurement hardware suitable for measuring the impedance time series comprising motion signal 1600 (e.g., as described in relation to FIG. 13-15), the synchronizing indication described in relation to FIG. 15 (e.g., an ECG signal, as described in relation to FIG. 13-14), and/or a motion tracking system capable of tracking the 3-D spatial position of a probe carrying one or more electrodes used to measure the impedance time series.

Some of the types/applications build on simpler types/applications. Motion signal representation 1600 includes the impedance measurements from the time series selected using the synchronizing indication, optionally normalized, labeled, or otherwise pre-processed for a stage of characterizing the position of the intracardial structure. Several of the blocks of FIG. 16 (for example, blocks 1601, 1602, 1605, 1606, 1607, 1609) represent processing blocks which convert motion signal representation 1600 into an output characterizing the position of the intracardial structure.

In a group of simple embodiments (proximity detector 1601), the position may be characterized simply as being within “detectable proximity” (for some criterion/criteria of detection and/or proximity) to an intracardiac electrode used to measure the target motion signal. The motion signal itself may comprise one or more phases, and “detectable proximity” may comprise identifying that the (optionally pre-processed) impedance measurement time series is consistent with the presence of the motion signal in some portion of these phases.

For example, there are described in relation to FIGS. 13-14 a number of waypoints corresponding to phases in the motion of the leaflets of the RAV; and in the time elapsing between waypoints, general trends of impedance increases/decreases are described. In some embodiments, slopes of impedance change are determined from data measured during times between these waypoints, according to a suitable method—for example, average slope or maximum slope amplitude is used, optionally limited to within a sub-portion of the time elapsing between waypoints.

The slopes, thus calculated, are optionally collected into vectors which are a reduced-component representation of the motion signal. For example (referring again to FIG. 14), vectors comprising a slope value for each period between the waypoints comprising the time of the right side of box 91, the time of the right side of box 92, the time of mark 93, the time of mark 95, and the time of the right side of the box 91 corresponding to the next heartbeat cycle. This would result in a four-component vector, referred to herein as a “proximity vector”. It should be understood that there are other metrics used, in some embodiments, to generate a reduced-component representation of the motion signal, for example using peak-to-trough amplitudes, spectral power, average amplitude, and/or another metric. In the degenerate case where a reduced-component representation of the motion signal may have just one component, it may be treated as a scalar. It is noted that the proximity vector itself comprises a characterization of motion of the moving structure of interest.

There is no particular requirement that the impedance time series measurements selected as demonstrating the motion signal be actually isolated from their surrounding measurements as part of the selecting. For example, in some embodiments, they may be labeled and/or time-normalized to fit within the input requirements of a signal detecting algorithm, using the synchronizing indication(s). There is no particular requirement that the impedance time series measurements are converted to a reduced-component representation of the motion signal. For example, in some embodiments, an unreduced time series of impedance measurements is provided as input to the result of a machine-learning process. The machine learning process optionally comprises training of a neural network algorithm using a training set comprising impedance measurement time series labeled (e.g., manually labeled) as having or not-having a detectable level of the relevant motion signal within them.

The criterion/criteria for “detectable” may be variously set. This may be conceptualized in the case of a reduced-component vector representation of the motion signal as defining a surface within the vector's parameter space which defines the threshold between “detected” and “undetected”. For example, it may simply be required that all vector components display the expected slope polarity (e.g., as described in relation to FIG. 14) for their timing position. Optionally, minimum amplitude of the slope larger than zero is also required for one or more of the vector components. The criteria for detectability may additionally be supplemented, e.g., by a suitable scheme of component weighting.

The output of the proximity detector 1601 optionally comprises a proximity indication 1601A. The proximity indication is optionally presented to a user via a computerized user interface (UI), and/or use as an input to further processing. For a user, simply knowing that a certain moving structure is in detectable proximity may be used, for example, to guide a probe to or away from a general location.

More spatial specificity (proximity mapper 1602) may potentially be gained by assigning the detectable/undetectable result to a particular position in space. In some embodiments, the electrodes used to measure the impedance time series are positioned on a probe (a catheter probe, for example), and the probe is moved around within a lumen of the heart (e.g., the right atrium, to characterize the position of the RAV). The spatial (three-dimensional) position of the probe may be estimated at any particular moment by a tracking system, for example, a tracking system based on the measurement of alternating electrical fields extending through the cardiac lumen; optionally using electrodes of the probe itself.

The tracking data 1603 are received, and associated moment-by-moment with the current determination as to whether the structure of interest is detectable or not. This results in an output of spatially defined locations (proximity map 1604) of “locations near the structure” (e.g., a point cloud, wherein each point is defined in three spatial dimensions). The aggregate of these locations are optionally used as a proxy loosely characterizing the position of the structure itself. Proximity map 1604 is optionally displayed via computer user interface (UI), and/or otherwise used as an input for further processing. This comprises a form of imaging; the proximity map 1604 being considered as representing data in a spatial format which may be provided for display as an image.

For somewhat more resolution (relative proximity detector 1605), the locations near the moving structure of interest may be characterized in terms of their relative magnitude (or other measure of “closeness” to the structure), rather than merely in terms of passing a threshold. Magnitude may be calculated, for example, as the proximity vector magnitude, optionally weighted differently for different components of the proximity vector.

The proximity magnitude may be used, e.g., to generate a relative proximity indication 1605A. This may be provided to a computerized user interface (UI), and/or used as an input to further processing, for example, to relative proximity mapper 1606. Relative proximity mapper 1606 works substantially as does proximity mapper 1602, but using the relative proximity indication 1605A.

The output relative proximity map 1606A is optionally displayed on a computerised user interface (UI) as a point cloud resembling a heat map—the closer to the structure of interest, the “hotter” (e.g, as indicated by display color, transparency, and/or size) the probe position. It should be noted that this type of embodiment has the property of using spatial position resolution available for a first measurement modality (used to track positions of a measurement probe) that helps to refine spatial position information which the measurement probe collects from a structure remote from its own position. This may also be referred to as mixed-modality imaging; the map 1606A being considered as representing data in a spatial format which may be provided for display as an image.

In some further embodiments (distance estimator 1607), the motion signal representation 1600 is more particularly converted to an estimated distance (preferably in a particular direction) from the position of the probe at the time of measurement. For example, for the relative proximity detector, a small motion signal may indicate that the structure is further away, and a larger motion signal may indicate that the structure is closer. The distance may be converted to a calibrated distance indication 1607A by one of a number of potentially suitable methods.

One general class of method comprises generating (via actual measurement and analysis) a number of motion signal representations while tracking positions of the probe doing the measurements, and also obtaining an actual position of the moving tissue structure of interest by a separate method; for example, measuring spatial positions at which the probe makes its nearest physical approach to the moving tissue structure (e.g., contact or near contact). In the case of leaflet valves, for example, care in the approach may be taken to avoid interfering with leaflet motions and/or damaging the leaflets. Using the actual position and the tracked probe positions, a correspondence between motion signal representation features and spatial distance/direction may be determined. This determination can be performed, e.g., via machine learning to produce a suitable machine-learned distance determination algorithm. The machine-learned distance determination algorithm may then in turn be applied to subsequent on-line measurements in order to estimate distance without necessarily knowing the actual position of the moving tissue structure in advance.

Where a sufficient number of data samples are being gathered during a particular procedure, the machine learning phase is optionally supplemented and/or replaced by a more theoretically explicit method which adjusts fitting parameters to minimize the error in the estimated positions of the moving tissue structure (accounting for the phase of the motion). For example, during moments when a RAV is closed, measurements from all Z coordinates above a certain X-Y coordinate pair should result in a same estimated position of the valve leaflet portion below it. Any of several parameterized functions may serve to sufficiently fit the data; for example, a polynomial including an inverse-power term relating signal magnitude to distance. The exact function may also depend on details of pre-processing.

As also described for the other single-measurement output types, there may again be generated (by absolute location mapper 1609) a mapped representation of distances of the moving structure as a function of measurement position. Preferably, the mapped representation (structure map 1609A) is simply referred to estimated absolute positions of the moving structure, offsetting the known position of the probe by the estimated distance of the moving structure. The direction of the moving structure is generally known from the measurement configuration, but may additionally or alternatively be calculated based on the direction in which the motion signal amplitude is increasing.

Optionally, structure map 1609A is time resolved—with different positions represented for different times of, e.g., a phasic movement of the moving tissue structure. The time resolution is optionally obtained by analyzing amplitudes of various components of the motion signal representation, and/or by application of a movement model which converts, e.g., a certain time-averaged position of the moving tissue structure (optionally as a function of X-Y position, for example over a cardiac valve annulus) into an estimated sequence of positions over the course of a heartbeat cycle.

It should be understood that any individual measurement of “distance” between an electrode in a particular place and a moving tissue structure is a kind of weighted average distance. For example, in the case of an RAV, the motion signal measurement is influenced not only by distances of the leaflet portion immediately “below” it (in the X-Y plane), but also by distance of leaflet portions further away. In effect, the signal measurement is “blurry”. Optionally, this is accepted, and structure map 1609A is understood to correspondingly blur the reconstructed positions of the mapped moving structure.

However, the blurring is, in some embodiments, deconvolved; e.g., by solving an inverse problem corresponding to the measurement conditions and results obtained. The inverse problem takes the form of calculating what configuration of valve leaflets (comprising their shape and optionally motions) could have resulted in the measurement results obtained, given what else known about the measurement conditions (constraints). The inverse problem may be solved, for example, by a gradient descent method which seeks to reduce the error between theoretically calculated impedance amplitude effects (e.g., according to Maxwell's equations) of membranous valve leaflets in certain positions with measurements actually obtained.

Any particular embodiment of structure map 1609A may also be referred to as a product of mixed-modality imaging; the structure map 1609A being considered as representing data in a spatial format which may be provided for display as an image.

### Systems for Motion-Synchronized Impedance Measurement Time Series Data

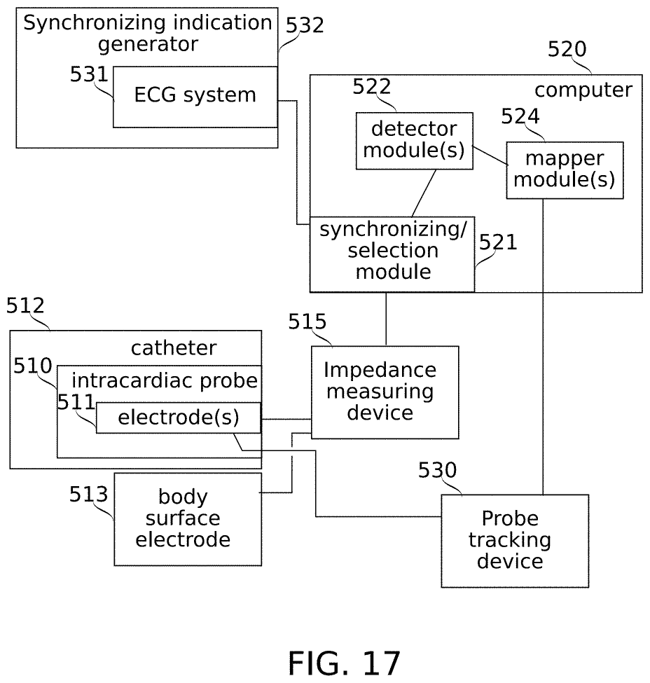

Reference is now made to FIG. 17, which is a schematic representation of a system for measuring the position of a moving intracardiac tissue structure, according to some embodiments of the present disclosure.

ECG system 531 is a system comprising electrodes, measurement, and processing hardware suitable to generate an ECG, for example as shown and described in relation to FIG. 13-14. ECG system 531 is a particular instance of a synchronizing indication generator 532, which may additionally or alternatively comprise a heart sound detector, ultrasound device, pulse pressure detector, or another device for measuring a synchronization indication, for example as described herein.

Intracardiac probe 510 (e.g., a probe insertable to a heart over a catheter 512) comprises one or more electrodes 511, which are configured to make intracardiac measurements of impedance in a time series (e.g., at a sampling rate of about 100-500 Hz), via impedance measuring device 515. Body surface electrode 513 is optionally used as the reference ground for the one or more electrodes 511.

Computer 520 is configured to receive the synchronizing indication of block 532, impedance measurement time series produced from impedance measuring device 512, and to apply to them the method of FIG. 15, using synchronization and selection module 521.

There may optionally be provided one or more detector modules 522 (corresponding, for example to blocks 1601, 1605, and/or 1607 of FIG. 16), and/or one or more mapper modules 524 (corresponding, for example, to blocks 1602, 1606 and/or 1609 of FIG. 16). In some embodiments, mapper modules 524 receive spatial tracking information about intracardiac probe 510 (for example, as also measured from one or more of electrodes 511) via probe tracking device 530.

### Heart Wall Characterization Using Impedance Signals Due to Motion

Reference is now made to FIGS. 11A-11D, which show four graphs, each of time development of impedance measured by an electrode from one wall of a right atrium (RA) of a pig, as labeled.

In each graph, each light line represents time development measured by one pair of electrodes during one heartbeat. The thick lines 1110, 1120, 1130, 1140 represent an average of all the light lines 1111, 1121, 1131, 1141. The graphs show only measurements obtained from electrodes identified to touch the wall during the measurement. An electrode was considered to touch the wall only if the variance in impedance measured over the heartbeat by that electrode was larger than 75% of the mean variance for the heart wall to which it was assigned. The four walls of the right atrium, from which the measurements were taken, are: a RA wall bordering the LA (FIG. 11A); the lateral RA wall (FIG. 11C), the anterior LA wall (FIG. 11C), and the wall between the RA and the aorta (FIG. 11D). The X axis is time, represented in units of seconds after normalization to a heart rate of 80 beats per minute. Thus, each line shows the time development of impedance measured during a single heartbeat, regardless of the animal's heart rate. A heartbeat may be defined using a body surface electrocardiogram taken simultaneously with the impedance data. Along the Y axis, impedance values are given in Ohms.

The graphs shown in FIGS. 11A-11D exemplify reference impedance data. The figure shows impedance measurements received during heartbeats. The X axis is time, from half a second before a QRS peak to half a second after a QRS peak, normalized to 80 beats per minute.

In some embodiments, a heart wall is identified by accessing impedance data collected during a single heartbeat. The impedance data may include impedance measurements from a plurality of electrode pairs. For example, if the data is measured using a probe comprising 10 electrodes (e.g., a Lasso™ catheter), and impedance is measured between each pair of adjacent electrodes. Accordingly, 9 time series, one for each electrode pair, is measured. These time series are then compared to the data presented in FIGS. 11A-11D, to determine the wall that the electrodes touched during the measurements. The comparison may be to the thick lines 1110, 1120, 1130, 1140 in FIGS. 11A-11D, or to any other data structure including and/or summarizing a population of one or more of the light lines 1111, 1121, 1131, 1141. Optionally, a machine learning algorithm is used in order to carry out the comparison, with the learning inputs comprising data corresponding to light lines 1111, 1121, 1131, 1141. Optionally, a learning algorithm learns to distinguish between one wall (e.g., the LA) and all the others. There may be a plurality of such algorithms (e.g., one for each wall). Optionally each algorithm is run on any given input data. Identifications consistent with a plurality of different walls are optionally interpreted as indicating that the position was intermediate between those different walls.

In some embodiments, only measurements from electrodes that touched the heart wall with force above a predefined contact force (or contact quality) threshold are considered. The contact force or quality may be determined using methods known in the art, for example, as mentioned hereinabove. Similarly, in some embodiments, measurements made by an electrode pair during a heartbeat are considered only if the mean variance of the measurements over the heartbeat is larger than a threshold. In some embodiments, heartbeats during which some or all of the electrodes don't actually touch the heart wall (e.g., vary by less than 75% of the mean variance) are excluded from consideration, both from measurement and reference data. In FIGS. 11A-11D, the light lines 1111, 1121, 1131, 1141 show results measured only with electrodes considered touching the heart wall.

In some embodiments, the impedance measured from each electrode pair is averaged over the heartbeat, and the distribution of the average impedances over the electrode pairs is used as a feature in the comparison. In some embodiments, one or more moments of this distribution are used in the comparison; for example, the average and/or the variance.

Reference is now made to FIG. 12, which schematically represents typical time courses of pressure 1210, 1212, 1214) an electrocardiogram 1216, and wall impedance signals 1218, 1220, 1222, 1224 measured at different walls of a heart, according to some embodiments of the present disclosure.

Time courses 1218, 1220, 1222, 1224 represent impedance time courses measured in contact with the left atrial, lateral, anterior, and aortic walls of the right atrium, respectively; for example as described for the thick lines 1110, 1120, 1130, 1140 from FIG. 11. They are extended to show more than a single heartbeat. The four upper lines are graphs showing time development of aortic pressure 1210, atrial pressure 1212 and ventricular pressure 1214 during the same portions of heartbeats, and an electrocardiogram 1216. The heartbeat portions may be identified using the electrocardiogram, as known in the art of electrophysiology.

In some embodiments, rather than comparing an entire measured data segment to an entire reference data segment, the wall is identified using a comparison made between slopes and/or amplitudes of the lower four lines 1218, 1220, 1222, 1224 at specific time-points (also referred to herein as stages) along the heartbeats. In some embodiments, the specific times at which the slopes are used for comparing the measured data to the reference data are shown in FIG. 12 as vertical lines 1201-1204. Line 1201 marks a time of mitral valve closing, and line 1202 marks a time of aortic valve opening. Line 1203 marks a time of aortic valve closing, and line 1204 marks a time of mitral valve opening. Openings/closings through two successive heartbeats are shown.

Additionally or alternatively, in some embodiments, the specific times are set arbitrarily, e.g., at equal time distances along a heartbeat. The number of slopes considered for each electrode during a heartbeat may vary among embodiments, for example, from 1 to about 20; preferably between 4 and 10: for example, 6 or 7. Additionally or alternatively, slope and amplitude may be used at the time of the ORS complex, or any other time stage that can be identified based on the ECG.

### Valve Leaflet Mapping Using Impedance Measurements

Reference is now made to FIG. 19, which is a schematic flowchart of a method of mapping valve leaflets, according to some embodiments of the present disclosure.

At block 1902, in some embodiments, a valve plane estimation is accessed, which defines an approximate mapping region from within which measurements of the valve leaflets to be mapped are made. The valve plane estimation is not necessarily itself planar; e.g., it may have a thickness, and is not necessarily defined as geometrically flat (e.g, it may be cupped or otherwise adapted to accommodate radial differences in the normal positioning and/or range of movements of the valve leaflets).

The method of determining the valve plane estimation may be, for example, as described in relation to FIG. 18, or another method described in relation to block 116 of FIG. 1. This method takes advantage of electrophysiological signal differences in measurement made in different locations (e.g., along an imaginary axis extending approximately orthogonally through the valve plane) to help determine where the valve plane is. Optionally, a suitable valve plane is determined by another method; e.g., based on the characteristic geometric shape of the valve annulus.

The region defined for making measurements of the valve leaflets is optionally offset from the originally accessed valve plane estimation along an imaginary axis extending transversely to the valve, e.g., by up to about 10 mm. Greater distance from the valve leaflets will tend to reduce the impedance signal due to leaflet motion, but being too close may result in leaflet contact during measurements which potentially interferes with mapping; e.g., by limiting motion of the valve leaflets themselves.

The use of a reference region provides a potential advantage by its use to confine impedance measurements of a structure to positions which are neither too far away (so that the impedance signal due to their proximity is too weak to detect and/or isolate), nor too close (e.g., so that the measuring probe itself interferes with the shape and/or movement of the structure). There is also a potential advantage for use of the reference region insofar as it helps to ensure that all the measurements which are used in the imaging can be treated as equivalent; or, optionally, to as deviating from equivalency in a manner which can be systematically corrected for (e.g., by normalizing amplitudes as a function of distance from the reference region). A further potential advantage arises insofar as measuring at a controlled distance relative to the structures being characterized helps to make measurement results more comparable between different subjects, and/or more reliably similar (e.g., similar to the eyes of a doctor interpreting the results).

For simplicity of description, algorithm descriptions for operations of FIG. 19 assume the condition that the measurements are obtained from a single (e.g., atrial) side of the valve leaflets being mapped. However two-sided leaflet mapping is optionally performed wherein measurement results from either side of the valve are placed in correspondence by a suitable transformation. Two-sided mapping is described, for example, in International Patent Publication No. WO2020/008416S, the contents of which are included herein by reference in their entirety.

At block 1903, in some embodiments, impedance measurements are accessed, each measurement being associated with a corresponding spatial position. Heart valve leaflets have a significantly higher impedance than blood. Accordingly, as they approach an electrode, the impedance it measures goes up; as they recede from the electrode, the impedance it measures goes down.

In some embodiments, the impedance measurements are pair impedances; that is, involving measurements for intralumenal electrodes considered pairwise. Optionally, for each individual electrode of the pair, impedance is measured to a ground electrode, e.g., a body surface ground. Alternatively, the impedance is measured between the intralumenal electrodes, e.g., with one of the electrodes serving as the ground reference. Optionally, impedance is measured between each of two intraluminal electrodes and a common ground to provide a pairwise impedance. Optionally, two such pair wise impedances, measured from two pairs having a common intraluminal electrode are averaged to provide an impedance value that may be treated as if measured at the common intraluminal electrode. The measurements are referred to herein as being made at “positions over the valve”, in the sense that both the measurement position and a corresponding valve leaflet position indicated by the measurement roughly share x, y coordinates in some coordinate frame, but are offset from each other along a z axis dimension, in the direction of the atrium. In the case of the tricuspid valve, for example, the z axis corresponds to the imaginary AV axis, e.g., as described in relation to FIG. 18.

Preferably, the impedance measurements are also associated with time-of-measurement data. The time-of-measurement data, furthermore, is optionally indexed and/or indexable to the phase time during the heartbeat at which the impedance measurements were taken. The indexing may comprise, for example, comparing time of measurement to the timing of characteristic features of a simultaneously recorded ECG (body surface and/or intracardially recorded). Additionally or alternatively, and insofar as the impedance measurements themselves reflect periodic movement of the heart, the impedance measurements may serve as their own phase reference. However, since the impedance measurements are being taken while the electrodes are moving around, comparison to reference recording may be more stable as an indication of heartbeat phase as such.

Optionally, the impedance measurements of block 1903 include impedance measurements made concurrently with electrophysiological measurements used to establish the location of the valve plane estimation 1902.

The further operations described in relation to FIG. 19 are for embodiments in which heartbeat phase assignments are available for each of the impedance measurements. However, valve leaflet mapping is optionally performed (with loss of temporal specificity) without use of time-of-measurement data, e.g., using population statistics of the impedance measurements as a marker for valve movement. For example, a standard deviation of measurements taken from a location is expected to be relatively low when the location is always distant from moving portions of the leaflets, compared to a location to which the leaflets move into successively closer and further proximity during a heartbeat.

Impedance measurements 1903 are optionally accessed as they are being obtained (e.g., new measurement points are iteratively accessed and integrated into the leaflet map), and/or accessed as a block of previously acquired measurements. The position from which each impedance measurement 1903 is taken is optionally considered to be the mean position of intralumenal electrodes being used in the measurement.

At block 1904, in some embodiments, the impedance measurements from block 1903 are gated, based on the proximity of their associated spatial position to the valve plane estimated position of block 1902. Accordingly, the impedance measurements from block 1903 are treated in some embodiments differently if associated with spatial positions near or far from the valve plane estimated position of block 1902.

Alternatively, the gating may be based on the associated spatial position of the impedance measurement being within a region defined based on the valve plane estimation. Proximity (or region extent) is optionally set according to a threshold distance of, e.g., about 1-5 mm between the estimated valve plane position and the impedance measurement-associated spatial position.

At block 1906, in some embodiments, impedance measurements which pass the proximity gate of block 1904 are assessed as potential indications of valve leaflet position.

In some embodiments, at block 1908 (within block 1906), impedance measurements are grouped, windowed, and/or weighted according to their phase timing. For example, a whole heartbeat cycle may be divided into 20-30 temporal bins, and measurements assigned to each bin according to their own timing. Alternatively, a sliding time window function may be defined to allow use of impedance measurements according to if and where they fall within the time window. The window function may apply a weighting, e.g., reducing the influence of an impedance measurement on a result calculated for the windowed time period according to increased temporal distance from a center of the window. Windows can be overlapping or separate. The effective duration (width) of the window is optionally itself dynamic as a function of heartbeat phase; for example, short when valve movement is rapid (to avoid losing movement detail), but longer when valve movement is slow (increasing integration time and potentially reducing sampling noise). Each window position and/or bin is also referred to as defining a “frame”, within which valve leaflet position is characterized as if for a single moment. Valve leaflet position changes from frame to frame.

At block 1910 (also within block 1906), valve positions at a plurality of locations are estimated using the value of the impedance measurement as a proxy for valve distance from the measurement location. Broadly, a higher impedance is treated as indicating greater proximity of a leaflet to the measurement position, while a lower impedance indicates a greater distance. Optionally, all measurements passing the gate of block 1094 are considered (for purposes of analysis) as belonging within a single plane of measurement voxels (e.g., a single “z” coordinate), with their off-axis locations (e.g., “x” and “y” coordinates) corresponding to different respective parts of the valve leaflets sharing the same x and y coordinates (at the heartbeat phase of the measurement). Expressed another way, determination of distance is optionally simplified to threshold—either impedance reaches the threshold for some measurement at some point in time, or it doesn't. If it does, then the leaflet is “at” the measurement position (or at a close position somewhat offset from the measurement position). If it doesn't then the leaflet is “away” from the measurement position.

Optionally, the distance indicated by impedance (and/or the impedance itself) is adjusted slightly depending on whether the measurement was taken exactly on the valve plane, a little above it (tending, e.g., to reduce the impedance actually measured), or a little below it (tending, e.g., to increase the impedance actually measured). With larger distances, finding a correct magnitude of adjustment may be more difficult, since the relationship between distance and impedance is potentially quite non-linear, and correspondingly difficult to correct without introducing new systematic distortions. Small adjustments may remain within an “approximately linear” range where the calibration is less likely to lead to misleading distortions. To determine calibration values, differences in measurements at the same phase time from positions in about the same x-y position (but slightly different z positions) may be used as reference.

Threshold-based analysis methods have a potential advantage, because the impedance signal tends to rise quite sharply as an inverse function of distance between measurement electrode and tissue. Whatever other signals may be influencing the impedance measurement at the same time will, accordingly, tend to be more easily disregarded, when the leaflet-to-electrode distance is smallest. Nevertheless, in some embodiments, threshold-based determinations of valve leaflet proximity to the measurement position are replaced with and/or augmented by graded representations of leaflet position. As an example of augmentation in particular: the rising and/or falling phases of an impedance signal as it approaches and recedes from the threshold value may be used to estimate leaflet distance at time other than those that actually cross the threshold.

At block 1912, in some embodiments, valve position is converted to a display form (and displayed).

In image representations of a leaflet map, the x and y coordinates are optionally binned at some resolution to create voxels (e.g., samples within every square millimeter are averaged). Alternatively, a spatial windowing function is used to group measurements according to position (e.g., the value used for each represented point is comprised of a window-weighted average of measurements near the point).

There is no particular limitation on plotting resolution vs. measurement sampling density. For plotting of x, y locations, interpolation from neighboring x, y coordinates for which measurements are available may be used. For example, every x, y location for which a location is to be plotted is optionally generated by averaging the values of the three nearest measurements in x,y coordinates. Measurements used in the average are optionally weighted inversely according to distance from the plotted location. Where sampling density is similar or greater than plotting density, it may be feasible to use another interpolation method; for example, a bilinear, quadratic, or other interpolation method. Interpolation is optionally performed through the dimension of heartbeat phase time and/or through spatial dimensions.

In some embodiments, the range of impedance values measured is assigned to a range of colors, and colors plotted across a “valve plane” representation accordingly. In some embodiments, the color is bivariate according to a threshold criterion. Two different colors may be chosen to represent “further” and “closer” to the valve plane. One of the colors may be “transparent” (e.g., in the instance of a purely threshold-based analysis, where the leaflets are treated as present or not at any given moment and position). In this case, displaying a sequence of mapping results (throughout a heartbeat cycle phase) as a cine may result in a changing pattern that shows valves closing and opening. In the cine, an opening, receding valve will appear as leaflet areas that “shrink”, while a closing, approaching valve appears as leaflet areas that “grow”, potentially merging into a single area indicative of the closed valve. Regions which never have any super-threshold valve indication appearing in them may indicate areas of incomplete coaptation associated, e.g., with valve regurgitation (assuming that those regions are also sampled regions, and not simply holes in the data sampling region).

The threshold, in embodiments where one is used, is optionally set, for example, as an absolute value, as a percentile of potentially relevant measurements, or as a magnitude of deviation of a particular measured impedance from an average value—optionally average for the whole valve, or average for some particular region of the valve. Adjusting the threshold may give the impression of viewing the valve at different cross-sectional levels. Optionally, there are provided user interface controls that allow a user to adjust threshold levels until features of particular interest (e.g., regions of poor coaptation) are seen most clearly. A 3-D display of a stack of images generated for different thresholds may give an impression of the shape of the valve (albeit, potentially distorted due to non-linearities in the relationship of impedance to distance). It may be noted that an impression of a “tilt”, cupping, or other distortion in the cross-sectional plane may be introduced by using different thresholds on different portions (e.g., different sides and/or different radial positions) of the valve plane. Optionally, user interface controls are provided to adjust the threshold, e.g., linearly across a chosen direction of the plane. This may help to bring all leaflets into an equivalent view, even if the original valve plane estimate is, for some reason, skewed relative to the valve leaflets themselves.

Optionally, a range of multiple colors (e.g., 256 colors or grayscale values) is mapped to a range of impedance measurement values, and the plotting color is selected according to where impedance values fall within that range. In some embodiments, impedance is converted to movements along a z-axis, simulating movement of the valve itself. This may be implemented, e.g., by an image-stack method, or by assigning z-axis values according to a selected (and optionally calibrated) impedance-to-distance function. The distance assignment is optionally selected according to a range which is simply suitable for emphasizing structure in the screen display, or it may be calibrated to actual distances of valve movement (relative to other heart features optionally shown), e.g., by mapping the range of impedance values measured to a typical range of valve leaflet positions.

In some embodiments, what is plotted (e.g., as a color) is the phase time at which each valve leaflet area (corresponding to a measurement position over the valve) crosses a certain threshold level (by going above it and/or falling below it). This has the potential advantage of emphasizing difference between nearby regions (that might be due to gaps or edges), while also allowing values to be plotted for a large portion of the valve within a single, optionally 2-D image.

Optionally, what is plotted is a statistical measure of a population of impedance measurements over time, e.g., a magnitude of the minimum-to-maximum impedance value for a position over the valve, a standard deviation of impedance value for a position over the valve, or another statistical metric.

For any of these view type, skews and offsets may be introduced as also described for the binary threshold views, to allow a user to optimize the leaflet view being presented for features of particular interest to them. These parameters are optionally controlled via user interface controls, and/or adjusted automatically. A method of automatic adjustment, in some embodiments, comprises adjusting parameters of a threshold function defined over an x, y plane until areas displayed as valve leaflet regions appear about equal in area, e.g., in the different quadrants of the valve leaflet map. Planar skew and offset are potentially relatively simple to adjust and interpret. Optionally, more complex “compensations” are supported, e.g., an adjustment term which is a function of radial distance.

It is noted that the estimate of the valve plane provided at block 1902 may itself be time-varying, e.g., moving along with the valve as the heart beats. Using a time-varying estimate of valve plane position provides a potential advantage by maintaining the gating criterion in a more constant spatial relationship with the structure being measured.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1. A system configured to identify structures of a body lumen for guidance of procedure actions within the body lumen, the system comprising a processor and memory storing instructions, wherein the processor operates according to the instructions to:
access measurements of:
impedance, measured using by one or more electrodes positioned within the heart, and
an electrophysiological signal indicative of electrical activity of heart tissue;
process the impedance to identify tissue according to the positioning of the one or more electrodes, using the electrophysiological signal; and
provide the identification as output for guidance of the procedure actions.

2. The system of claim 1, wherein the processor processes the impedance using timing information in the electrophysiological signal.

3. The system of claim 1, wherein the processor processes the impedance using positional information indicated by the electrophysiological signal.

4. The system of claim 1, comprising a display; wherein the processor provides the identification as output in the form of a portion of an image displayed on the display.

5. The system of claim 1, wherein the processor also:
operates according to the instructions to access measurements of spatial position of the one or more electrodes while the impedance was measured; and
uses the measurements of spatial position together with the impedance and the electrophysiological signal to identify the tissue.

6. The system of claim 1, wherein the impedance is indicative of motion of the identified tissue.

7. The system of claim 6, wherein the identified tissue comprises tissue of a heart lumenal wall identified based on the indications of its movement in the impedance.

8. The system of claim 7, wherein the indications of heart lumenal wall movement are identified based further on relative timing of the electrophysiological signal and the impedance.

9. The system of claim 7, wherein the identification categorizes the heart lumenal wall according to which wall of a heart chamber the at least one electrode is in contact with.

10. The system of claim 7, wherein the identification distinguishes interatrial septal wall from lumenal wall adjacent to the aorta.

11. The system of claim 1, wherein the impedance is indicative of motion of the identified tissue.

12. The system of claim 6, wherein the identified tissue comprises tissue of a heart valve leaflet, identified based on indications of motion of the leaflet in the impedance.

13. The system of claim 12, wherein the indications of motion of the heart valve leaflet are characterized using relative timing of the electrophysiological signal and the impedance.

14. The system of claim 12, wherein the indications of motion of the heart valve leaflet are characterized using positioning of the one or more electrodes relative to a region identified based on the electrophysiological signal.

15. The system of claim 14, wherein the region identified based on the electrophysiological signal is identified based on its position relative to a heart valve.

16. The system of claim 14, wherein the processor uses positioning of the one or more electrodes relative to a region identified based on the electrophysiological signal to select impedance measurements for use in the identification.

17. A method of identifying structures of a body lumen for guidance of procedure actions within the body lumen, the method comprising:
accessing measurements of:
impedance, measured by one or more electrodes positioned within the heart, and
an electrophysiological signal indicative of electrical activity of heart tissue;
processing the impedance to identify tissue according to the positioning of the one or more electrodes, using the electrophysiological signal; and
providing the identification as output for guidance of the procedure actions.

18. The method of claim 17, wherein the processing uses timing information in the electrophysiological signal.

19. The method of claim 17, wherein the processing uses positional information indicated by the electrophysiological signal.

20. The method of claim 17, comprising providing the identification as output in the form of a portion of an image displayed on the display.

21. The method of claim 17, comprising:
accessing measurements of spatial position of the one or more electrodes while the impedance was measured; and
using the measurements of spatial position together with the impedance and the electrophysiological signal to identify the tissue.

22. The method of claim 17, wherein the impedance is indicative of motion of the identified tissue.

23. The method of claim 22, wherein the identified tissue comprises tissue of a heart lumenal wall identified based on the indications of its movement in the impedance.

24. The method of claim 23, wherein the indications of heart lumenal wall movement are identified based further on relative timing of the electrophysiological signal and the impedance.

25. The method of claim 23, wherein the identification categorizes the heart lumenal wall according to which wall of a heart chamber the at least one electrode is in contact with.

26. The method of claim 23, wherein the identification distinguishes interatrial septal wall from lumenal wall adjacent to the aorta.

27. The method of claim 22, wherein the identified tissue comprises tissue of a heart valve leaflet identified based on the indications of its movement in the impedance.

28. The method of claim 27, wherein the indications of heart valve leaflet movement are characterized using relative timing of the electrophysiological signal and the impedance.

29. The method of claim 27, wherein the indications of heart valve leaflet movement are characterized using positioning of the one or more electrodes relative to a region identified based on the electrophysiological signal.

30. The method of claim 29, wherein the region identified based on the electrophysiological signal is identified based on its position relative to a heart valve.

31. The method of claim 29, comprising using the positioning of the one or more electrodes relative to a region identified based on the electrophysiological signal to select impedance measurements for use in the identification.

32-128. (canceled)
